(19) United States
(12) Patent Application Publication
(10) Pub. No.: US 2026/0091139 A1
(43) Pub. Date: Apr. 2, 2026

(54) TREATMENT OF CARDIOMYOPATHY WITH AAV GENE THERAPY VECTORS

(71) Applicant: DINAQOR AG, Schlieren (CH)

(72) Inventors: Peter Cameron COLOSI, San Anselmo, CA (US); Pooja AGARWAL, Novato, CA (US); Wesley YONEMOTO, Novato, CA (US); Sergio VIEDMA-GONZALEZ, Novato, CA (US); Eduard AYUSO, Zürich (CH); Thomas VOIT, London (GB); Valeria RICOTTI, London (GB)

(21) Appl. No.: 19/113,698

(22) PCT Filed: Sep. 22, 2023
(86) PCT No.: PCT/US2023/074831

§ 371 Date: Mar. 20, 2025

Related U.S. Application Data
(60) Provisional application No. 63/376,712, filed on Sep. 22, 2022.
(60) Provisional application No. 63/519,967, filed on Aug. 16, 2023.

(51) Int. Cl. A61K 48/00; A61K 38/17; A61P 9/10; C12N 15/86

(52) U.S. Cl. CPC A61K 48/0058; A61K 38/1719; A61P 9/10; C12N 15/86; C12N 2750/14143; C12N 2750/14152

(57) ABSTRACT

Provided herein are gene therapy compositions and methods of treating reduced levels of functional cardiac myosin binding protein C in a subject having hypertrophic cardiomyopathy.

### CROSS REFERENCE TO RELATED APPLICATIONS

This application claims the benefit of priority to U.S. Provisional Patent Application Nos: 63/376,712, filed Sep. 22, 2022, and 63/519,967, filed Aug. 16, 2023, each of which is incorporated herein by reference in its entirety.

### FIELD

Provided herein are recombinant adeno-associated virus (rAAV) gene therapy vectors and virus particles useful in the treatment and prevention of hypertrophic cardiomyopathy by increasing expression of cardiac myosin binding protein C (cMyBP-C).

### INCORPORATION OF SEQUENCE LISTING

This instant patent application includes a sequence listing in electronic format (Filename: PCT_SeqListing.xml; created Sep. 6, 2023; 763,166 bytes) and is incorporated herein by reference in its entirety.

### BACKGROUND

While considerable progress has been made in the prevention of heart diseases that are caused by environmental factors, such as nicotine, hypercholesterolemia or diabetes, and in the symptomatic treatment of heart conditions, there is still a need for methods that improve the treatment of inherited cardiomyopathies. Among the cardiomyopathies that are caused by genetic factors are hypertrophic cardiomyopathy (HCM), dilated cardiomyopathy (DCM), and arrhythmogenic right ventricular cardiomyopathy (ARVC).

Hypertrophic cardiomyopathy is the most prevalent genetic heart disease and is characterized by unexplained left ventricular hypertrophy. Hypertrophic cardiomyopathy is associated with initially normal systolic, but impaired diastolic function (Elliott et al., Eur. Heart J. 29: 270-6 (2008); Gersch et al., J. Thorac. Cardiovasc. Surg. 142: e153-203 (2011). Hypertrophic cardiomyopathy has a particularly high prevalence of about 1:500 in the general population (Maron et al., Circulation, 92: 785-9 (1995), and is the leading cause of sudden cardiac death in younger people, particularly in athletes. Although HCM is a life-threatening disease, no curative treatment exists to date (Carrier et al., Cardiovasc. Res. 85:330-338 (2010); Schlossarek et al., J. Mol. Cell. Cardiol. 50: 613-20 (2011).

Inherited hypertrophic cardiomyopathy is a genetic disease which is known to be caused by more than 1000 different mutations in at least 10 genes that encode components of the cardiac sarcomere, such as cardiac myosin binding protein C (cMyBP-C), β-myosin heavy chain (MYH7), cardiac troponin T (TNNT2), cardiac troponin I (TNNI3), myosin ventricular essential light chain 1 (MYL3), myosin ventricular regulatory light chain 2 (MYL2), cardiac a actin (ACTC), a-tropomyosin (TPM1), titin (TTN), four-and-a-half LIM protein 1 (FHL1) (Richard et al., Circulation, 107: 2227-2232 (2003); Schlossarek et al., J. Mol. Cell Cardiol. 50: 613-20 (2011); Friedrich et al., Hum. Mol. Genet. 21: 3237-54 (2012). Many mutations are missense mutations which encode full-length mutant polypeptides, while other frameshift or splice-site mutations may result in truncations (Marian et al., Circ. Res. 121: 749-70 (2017); Walsh et al., Genet. Med. 19: 192-203 (2017). The most common truncated mutant polypeptides are MYBPC3 and FHL1, which exhibit mainly frameshift mutations leading to C-terminal truncated proteins.

The most frequently mutated gene in HCM is MYBPC3 which encodes cardiac myosin binding protein C (cMyBP-C) (Bonne et al., Nat. Genet. 11:438-40 (1995); Watkins et al., N. Engl. J. Med. 364: 1643-56 (2011). cMyBP-C is a major component of the A-band of the sarcomere, where it interacts with myosin, actin and titin (Schlossarek et al., J. Mol. Cell. Cardiol. 50: 613-20 (2011). In humans and mice cMyBP-C is exclusively detected in the heart (Fougerousse et al, Circ. Res. 82: 130-3 (1998) and is involved in the regulation of cardiac contraction and relaxation (Pohlmann et al., Circ. Res. Circ. Res. 101: 928-38 (2007); Schlossarek et al., J. Mol. Cell. Cardiol. 50: 613-20 (2011). About 70% of the mutations in the MYBPC3 gene result in a frameshift and produce C-terminal truncated proteins (Carrier et al., Circ. Res. 80: 427-34 (1997). Truncated proteins are unstable and have never been detected in myocardial tissue of patients (Marston et al., Circ. Res. 105: 219-22 (2009); van Dijk et al., Circulation, 119: 1473-83 (2009); van Dijk et al., Circ. Heart Fail. 5:36-46 (2012).

Current drug-based treatments of HCM alleviate the symptoms but do not treat the genetic cause underlying the disease. A gene-based or RNA-based therapy would be the only curative treatment for HCM. Gene therapeutic approaches have successfully been tested in connection with non-genetic cardiac diseases (Jessup et al., Circulation, 124: 304-13 (2011).

### SUMMARY

The embodiments described herein relate to a vector construct, a recombinant replication deficient AAV particle, cells, and pharmaceutical compositions for delivering cardiac myosin binding protein C (cMyBP-C) to a subject with HCM or a subject with a deficiency in a functional cardiac sarcomeric protein such as cMyBP-C. The embodiments described herein also relate to the use of such AAV particles or such vector constructs to deliver a gene encoding cMyBP-C to the myocardium of such subjects.

The gene therapy vector is suitable for use in treating or preventing HCM in a mammalian subject in need of treatment, preferably a human subject. In some embodiments, the subject in need of treatment is one that carries a mutation in at least one or both genes encoding cMyBP-C. After administration into the subject to be treated, the vector provides for the expression of the encoded cardiac myosin binding protein in the subject, preferably in the myocardium of said subject.

In one aspect, the embodiments described herein provide a vector construct comprising a nucleic acid sequence that encodes a functional cMyBP-C protein. In one or more embodiments, the functional cMyBP-C protein comprises an amino acid sequence at least 90%, 95% or 98% identical to the amino acid sequence of SEQ ID NO: 2 (a human cardiac myosin binding protein C). In some embodiments, the functional cMyBP-C protein comprises an amino acid sequence at least 95% identical to the amino acid sequence of SEQ ID NO: 2. In example embodiments, the nucleic acid sequence encoding the functional cardiac myosin binding protein C is a wild-type sequence, of which SEQ ID NOs: 1, 42 and 43 are examples, or is codon optimized, or is a variant. Alternative codon optimized or variant human cardiac myosin binding protein C-encoding sequences are set out as SEQ ID NOs: 44-46. The coding sequence for cardiac myosin binding protein C (cMyBP-C) is, in some embodiments, codon optimized for expression in humans. In some embodiments, the nucleic acid sequence encoding the functional cMyBP-C is at least 85%, 90%, 95%, 96%, 97%, 98%, or 99% identical to any one of SEQ ID NOs: 1 or 42-46.

The protein to be expressed may also be a functional variant which exhibits a significant amino acid sequence identity (i.e., at least 60%, 65%, 70%, 75%, 80%, 85%, 90%, 95%, 96%, 97%, 98%, or 99%) compared to SEQ ID NO: 2. In this context, the term “functional variant” means that the variant of the cMyBP-C protein is capable of fulfilling the function of the naturally occurring cMyBP-C protein, e.g., providing structural and/or functional support to the sarcomere to restore normal myocardial contractility, and optionally is capable of suppressing expression of and/or reducing levels of, mutant cMyBP-C proteins or other mutant sarcomeric proteins.

Functional variants of a cMyBP-C protein may include, for example, proteins which differ from their naturally occurring counterparts by one or more amino acid substitutions, deletions or additions. For example, a variant protein of the human cMyBP-C protein of SEQ ID NO: 2 may have an amino acid sequence with at least 2, 3, 4, 5, 6, 10, or more, and/or up to 10, 20, 30 or more positions which have been substituted by another amino acid relative to SEQ ID NO: 2. As another example, a variant protein of the human cMyBP-C protein of SEQ ID NO: 2 may be truncated version of the human cMyBP-C protein. For example, the functional variant may be selected from the group consisting of the naturally occurring MYBPC3 splice variant lacking exons 5 and 6, termed variant 4 (as shown in SEQ ID NO: 46).

In one or more embodiments, the nucleic acid sequence encoding cMyBP-C is operably linked to one or more heterologous expression control elements. Preferably, expression of the cMyBP-C-encoding transgene is controlled by at least one cardiomyocyte-specific expression control element. Thus, in such embodiments, in the vector constructs described herein, the nucleic acid sequence encoding cMyBP-C is operably linked to a heterologous cardiomyocyte-specific transcription regulatory region. In some embodiments, in the vector constructs described herein, the expression control elements include one or more of the following: a promoter and/or enhancer; optionally an intron; optionally an exon; and a polyadenylation (polyA) signal. Such elements are further described herein.

The cardiomyocyte-specific transcription regulatory region may comprise one or more cardiomyocyte-specific expression control elements, such as a cardiomyocyte-specific promoter. Preferably the cardiomyocyte-specific promoter comprises at least a fragment or variant of the human cardiac troponin T (hTNNT2) promoter.

In some embodiments, the cardiomyocyte-specific promoter comprises a nucleic acid sequence at least, or more than, 80%, 85%, 90%, 95%, 97%, 98% or 99% identical to SEQ ID NO: 47 (over the full length of SEQ ID NO: 47). In some embodiments, the cardiomyocyte-specific promoter may be combined with an intron that enhances expression of the cMyBP-C protein, located 5′ to the cMyBP-C coding sequence. For example, the vector construct and/or AAV particle comprise, in 5′ to 3′ orientation, a cardiomyocyte-specific promoter, an intron that enhances expression of cMyBP-C protein, and a nucleotide sequence encoding cMyBP-C coding sequence. In some embodiments, the vector construct and/or AAV particle comprise (a) a cardiomyocyte-specific promoter comprising a nucleotide sequence at least 80% identical to any one of (i) SEQ ID NO: 47, (ii) SEQ ID NO: 48, (iii) SEQ ID NO: 49, (iv) SEQ ID NO: 50, (v) SEQ ID NO: 51, or (vi) SEQ ID NO: 52, (b) an intron comprising a nucleotide sequence at least 60% identical to SEQ ID NO: 53, located 5′ to the cMyBP-C coding sequence, a nucleotide sequence encoding cMyBP-C, and optionally a polyadenylation signal sequence. Alternatively, the intron comprises a nucleotide sequence at least 60% identical to SEQ ID NO: 56 or SEQ ID NO: 58.

In other embodiments, the cardiomyocyte-specific promoter may be combined with an intron that enhances expression of the cMyBP-C protein, located within the cMyBP-C coding sequence. In some embodiments, the intron sequence is located within the nucleotide sequence encoding cMyBP-C, for example, between any of the exons, e.g., between exon 2 and 3. In some embodiments, the intron is located at position 293 of any one of SEQ ID NO: 1 or 42-45.

In some embodiments, the vector construct and/or resulting AAV particle comprise a cardiomyocyte-specific promoter sequence that is a fragment or variant of the hTNNT2 promoter that is more than 420 and less than 544 nucleotides in length and that comprises a nucleic acid sequence at least 90% identical to SEQ ID NO: 47. In any of the embodiments described herein, the cardiomyocyte-specific promoter optionally excludes any one of SEQ ID NO: 1 to 85 of U.S. Pat. Pub. No. 2021/0252165.

For example, the cardiomyocyte-specific promoter sequence comprises a nucleic acid sequence at least, or more than, 80%, 85%, 90%, 95%, 97%, 98% or 99% identical to any of (i) SEQ ID NO: 47 or fragment thereof, (ii) SEQ ID NO: 48 or fragment thereof, (iii) SEQ ID NO: 49 or a fragment thereof, (iv) SEQ ID NO: 50 or a fragment thereof, (v) SEQ ID NO: 51 or a fragment thereof, or (vi) SEQ ID NO: 52 or a fragment thereof. In an example embodiment, the sequence of the cardiomyocyte-specific promoter comprises a nucleotide sequence at least 96%, 97%, 98%, or 99% identical to SEQ ID NO: 51. In some example embodiments, the sequence of the hTNNT promoter comprises at least nucleotides 1-106 and 507-532 of SEQ ID NO: 51, or at least nucleotides 507-532 of SEQ ID NO: 51, or at least nucleotides 521-532 of SEQ ID NO: 51.

In some embodiments, the vector construct comprises one or more introns that enhance expression of the cMyBP-C-encoding nucleic acid, e.g., such that increased levels are detectable in the myocardium or heart. In some embodiments, the intron comprises a globin intron, and/or a fragment or variant thereof or a chimeric intron, and/or a fragment or variant thereof. In one or more embodiments, the intron comprises a nucleotide sequence at least 60%, 65%, 70%, 75%, 80%, 85%, 90%, 95%, 97%, 98% or 99% identical to SEQ ID NO: 53. In one or more embodiments, the intron comprises a nucleotide sequence at least 60%, 65%, 70%, 75%, 80%, 85%, 90%, 95%, 97%, 98% or 99% identical to SEQ ID NO: 56. In one or more embodiments, the intron comprises a nucleotide sequence at least 60%, 65%, 70%, 75%, 80%, 85%, 90%, 95%, 97%, 98% or 99% identical to SEQ ID NO: 58. In some embodiments, the intron is inserted downstream from the promotor and 5′ to the cMyBP-C coding sequence. In some embodiments, the intron is located within the nucleotide sequence encoding cMyBP-C, for example between any of the exons, e.g., between exon 2 and 3. In example embodiments, the intron is inserted at nucleotides position 293 of the MYBPC3 wild-type cDNA sequence of SEQ ID NO: 1. In further example embodiments, the intron is inserted at nucleotides position 293 of the MYBPC3 wild-type cDNA sequence of any one of SEQ ID NOs: 42-45.

In some embodiments, the vector construct may further comprise an exon sequence or fragment thereof, preferably adjacent to an intron sequence, e.g., a globin intron adjacent to the 3′ end of a fragment of beta globin exon 3 (SEQ ID NO: 54). The cardiomyocyte-specific transcription regulatory region can comprise a combination of the intron and exon fragment, for example, SEQ ID NO: 55. In some example embodiments, the cardiomyocyte-specific transcription regulatory region comprises SEQ ID NO: 56.

In some embodiments, the vector construct comprises a polyadenylation signal, optionally a bovine growth hormone (bGH) polyA signal (e.g., SEQ ID NO: 59, 60, or 61) or fragment thereof, optionally a human growth hormone (hGH) polyA signal (e.g., SEQ ID NO: 62) or fragment thereof, optionally an SV40 polyA signal (e.g., SEQ ID NO: 63) or fragment thereof, optionally a Proudfoot synthetic polyA signal (e.g., SEQ ID NO: 65) or fragment thereof, or optionally a rabbit beta-globin polyA signal (e.g., SEQ ID NO: 66) or fragment thereof. In some embodiments, the polyA signal comprises a nucleotide sequence at least 90% identical to SEQ ID NO: 64. In some embodiments, the polyA signal comprises a nucleotide sequence at least 90% identical to SEQ ID NO: 59, for example, comprises SEQ ID NO: 60 or a fragment thereof, or SEQ ID NO: 61 or a fragment thereof. In example embodiments, the polyA signal is a fragment of SEQ ID NO: 62 is about 100 to about 500 nucleotides in length, or about 150 to about 400 nucleotides in length, or about 200 to about 300 nucleotides in length, or about 200 to about 250 nucleotides in length, that comprises SEQ ID NO 59.

In some embodiments, the vector construct comprises a nucleotide sequence at least 97%, 98% or 99% identical to any of SEQ ID NO: 3-41. In other embodiments, the vector construct comprises a nucleotide sequence at least 97%, 98% or 99% identical to a nucleotide sequence that is complementary to or is a negative (−) strand of any of SEQ ID NO: 3-41 or 92-169.

In some embodiments, the vector construct comprises a nucleotide sequence at least 97%, 98% or 99% identical to any of SEQ ID NO: 3, 6, 9, 12, 15, 18, 21, 24, 27, 30, 33, 36, or 39. In other embodiments, the vector construct comprises a nucleotide sequence at least 97%, 98% or 99% identical to a nucleotide sequence that is complementary to or is a negative (−) strand of any of SEQ ID NO: 3, 6, 9, 12, 15, 18, 21, 24, 27, 30, 33, 36, or 39.

In some embodiments, the vector construct comprises a nucleotide sequence at least 97%, 98% or 99% identical to any of SEQ ID NO: 4, 7, 10, 13, 16, 19, 22, 25, 28, 31, 34, 37, or 40. In other embodiments, the vector construct comprises a nucleotide sequence at least 97%, 98% or 99% identical to a nucleotide sequence that is complementary to or is a negative (−) strand of any of SEQ ID NO: 4, 7, 10, 13, 16, 19, 22, 25, 28, 31, 34, 37, or 40.

In some embodiments, the vector construct comprises a nucleotide sequence at least 97%, 98% or 99% identical to any of SEQ ID NO: 5, 8, 11, 14, 17, 20, 23, 26, 29, 32, 35, 38, or 41. In other embodiments, the vector construct comprises a nucleotide sequence at least 97%, 98% or 99% identical to a nucleotide sequence that is complementary to or is a negative (−) strand ofany of 5, 8, 11, 14, 17, 20, 23, 26, 29, 32, 35, 38, or 41.

Example embodiments include the following:

Construct C1 is 4950 bp in length (SEQ ID NO: 29) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (130 bp) (SEQ ID NO: 71). In further embodiments, Construct C1 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C2 is 4801 bp in length (SEQ ID NO: 32) and comprises the following elements in 5′ to 3′: 5′ AAV2-ITR (130 bp) (SEQ ID NO: 67), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (130 bp) (SEQ ID NO: 71). In further embodiments, Construct C2 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C3 is 4801 bp in length (SEQ ID NO: 35) and comprises the following elements in 5′ to 3′: 5′ AAV2-ITR (130 bp) (SEQ ID NO: 67), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 71). In further embodiments, Construct C3 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C4 is 4950 bp in length (SEQ ID NO: 38) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (130 bp) (SEQ ID NO: 71). In further embodiments, Construct C4 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C5 is 4950 bp in length (SEQ ID NO: 41) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (130 bp) (SEQ ID NO: 71). In further embodiments, Construct C5 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A1 is 5074 bp in length (SEQ ID NO: 5) and comprises the following elements in 5′ to 3′: 5′ AAV2-ITR (130 bp) (SEQ ID NO: 67), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (130 bp) (SEQ ID NO: 71). In further embodiments, Construct A1 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A2 is 4939 bp in length (SEQ ID NO: 8) and comprises the following elements in 5′ to 3′: 5′ AAV2-ITR (130 bp) (SEQ ID NO: 67), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 71). In further embodiments, Construct A2 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A3 is 4939 bp in length (SEQ ID NO: 11) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (130 bp) (SEQ ID NO: 71). In further embodiments, Construct A3 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A4 is 4939 bp in length (SEQ ID NO: 14) and comprises the following elements in 5′ to 3′: 5′ AAV2-ITR (130 bp) (SEQ ID NO: 67), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (130 bp) (SEQ ID NO: 71). In further embodiments, Construct A4 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A5 is 4871 bp in length (SEQ ID NO: 17) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (130 bp) (SEQ ID NO: 71). In further embodiments, Construct A5 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A6 is 5002 bp in length (SEQ ID NO: 20) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 71). In further embodiments, Construct A6 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A7 is 4781 bp in length (SEQ ID NO: 23) and comprises the following elements in 5′ to 3′: 5′ AAV2-ITR (130 bp) (SEQ ID NO: 67), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 71). In further embodiments, Construct A7 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A8 is 4844 bp in length (SEQ ID NO: 26) and comprises the following elements in 5′ to 3′: 5′ AAV2-ITR (130 bp) (SEQ ID NO: 67), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 71). In further embodiments, Construct A8 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

In any of the foregoing embodiments, the vector construct comprises at least one ITR sequence. Example ITR sequences include but are not limited to SEQ ID NOs: 67-74 including any complementary sequences and/or combinations thereof.

In any of the foregoing embodiments, the length of the vector insert beginning at one ITR and ending with the second ITR is between about 4 kb to about 5.5 kb in size. In one or more embodiments, the vector construct is an AAV vector genome about 4 kb to about 5.4 kb in size, about 4.5 kb to about 5.5 kb in size, or about 4.8 kb to about 5.2 kb in size, or about 4.5 kb to about 5 kb in size.

The vector construct is preferably a recombinant AAV vector construct. In some embodiments, the vector construct comprises (a) one or both of (i) an AAV 5′ inverted terminal repeat (ITR) and (ii) an AAV 3′ ITR; (b) a promoter and/or enhancer, e.g., a cardiomyocyte-specific transcription regulatory region; and (c) a nucleic acid sequence encoding a functionally active human cMyBP-C protein. In some embodiments, the vector construct comprises (a) an AAV 5′ inverted terminal repeat (ITR) sequence; (b) a promoter and/or enhancer, e.g., a cardiomyocyte-specific transcription regulatory region; (c) a nucleic acid sequence encoding a functionally active human cMyBP-C protein; (d) an intron; (e) a polyadenylation signal; and (f) an AAV 3′ ITR. In some embodiments, the intron is downstream of the promoter and positioned 5′ to the cMyBP-C coding sequence, while in other embodiments, the intron is located between exons of the cMyBP-C coding sequence, e.g., between exon 2 and exon 3. In further embodiments, the vector construct comprises (a) an AAV′ 5′ inverted terminal repeat (ITR) sequence; (b) a promoter and/or enhancer, e.g., a cardiomyocyte-specific transcription regulatory region; (c) a nucleic acid sequence encoding a functionally active human cMyBP-C protein; (d) an intron; (e) and exon; (f) a polyadenylation signal; and (g) an AAV 3′ ITR. The AAV 5′ ITR and/or AAV 3′ ITR may be from a heterologous AAV pseudotype (which may or may not be modified as known in the art). In some embodiments, the 5′ ITR and 3′ ITR sequences are derived from AAV2 (e.g., SEQ ID NO: 67-70 and 71-74, respectively).

In any of the foregoing embodiments, the vector construct comprises a nucleotide sequence at least 80%, 81%, 82%, 83%, 84%, 85%, 86%, 87%, 88%, 89%, 90%, 91%, 92%, 93%, 94%, 95%, 96%, 96.5%, 97%, 97.5%, 98%, 98.5%, 99%, or 99.5% identical to any of SEQ ID NOs: 3-42 and 92-169 over the length of SEQ ID NOs: 3-42 and 92-169, respectively. In some embodiments, the vector construct is at least 97%, 98% or 99% identical to any of SEQ ID NOs: 3-42 and 92-169 over the length of SEQ ID NOs: 3-42 and 92-169, respectively. In specific examples, the vector construct comprises a nucleotide sequence at least 85% identical to any of SEQ ID NOs: 29, 32, or 41, or at least 95% identical to any of SEQ ID NOs: 35 or 38. In other examples, the vector construct comprises a nucleotide sequence at least 90% identical to any of SEQ ID NOs: 29, 32, or 41, or at least 98% identical to any of SEQ ID NOs: 35 or 38. Such vectors, for example, preferably comprise flanking ITRs, a nucleic acid sequence encoding a functionally active human cMyBP-C protein coding sequence, a cardiomyocyte-specific regulatory region, an intron, and a polyA signal.

In another aspect, provided herein is a recombinant adeno-associated virus (rAAV) particle comprising an AAV capsid and the vector construct as described in one or more of the embodiments herein. Any AAV capsids, e.g., AAV1-13, may be used. In some embodiments, the recombinant AAV (rAAV) particle used for delivering the cMyBP-C-encoding gene has cardiac tropism. In such embodiments, the rAAV comprises an AAV capsid with cardiac tropism, for example, an AAV9-type capsid at least 85%, 90% or 95% identical to SEQ ID NO: 75, or an AAV1-type, AAV6-type or AAV7-type capsid, or a variant of any of these, that exhibits cardiac tropism. In one or more embodiments, the AAV capsid is a capsid for which preexisting humoral immunity is reduced compared to AAV9, e.g., when evaluated by IVIG neutralization in vitro.

In another aspect, provided herein are methods for the production of an AAV particle, useful as a gene delivery vector, the method comprising the steps of: (1) providing a cell (e.g., a mammalian cell) one or more nucleic acid constructs (a) comprising a vector construct as described herein comprising a nucleic acid encoding cMyBP-C as described herein that is flanked by two AAV ITR nucleotide sequences; (b) a nucleotide sequence encoding one or more AAV Rep proteins which is operably linked to a promoter that is capable of driving expression of the Rep protein(s); (c) a nucleotide sequence encoding one or more AAV capsid proteins which is operably linked to a promoter that is capable of driving expression of the capsid protein(s); and (d) optionally genes encoding AAP and MAAP contained in the VP2/3; (2) culturing the cell defined in (1) under conditions conducive to the expression of the Rep and the capsid proteins; and, optionally (3) recovering the AAV particle. In some embodiments, the cell is a mammalian cell. In some embodiments, the mammalian cell is a HEK293 cell. Also provided herein are a population of rAAV particles produced by such methods.

In yet another aspect, provided herein are pharmaceutical compositions comprising the vector construct described herein or the rAAV particle or population of rAAV particles described herein, and a sterile pharmaceutically acceptable diluent, excipient or carrier.

In a further aspect, provided herein are methods of delivering a MYBPC3 gene to a mammalian subject. Such methods include methods of expressing myosin binding protein C in a mammalian subject comprising administering to the subject a composition comprising the vector construct described herein, the rAAV particle described herein, or the pharmaceutical composition described herein, thereby expressing the encoded myosin binding protein in the subject. Preferably, in such methods, the mammal is a human and the myosin binding protein C is functional human myosin binding protein C as described herein. Such methods include a method of expressing myosin binding protein C in cells of the myocardium of a mammal by administering an amount of the vector construct, rAAV particle or pharmaceutical composition effective to increase the level of myosin binding protein C expression in the myocardium of the mammal. Such methods also include a method of increasing the level of functional myosin binding protein C in the heart tissue (e.g., myocardiocytes) of a mammal by administering an amount of the vector construct, rAAV particle or pharmaceutical composition effective to increase the level of functional myosin binding protein C in the heart tissue (e.g., myocardiocytes) of a mammal. Such methods also include a method of treating a deficiency in functional wild type myosin binding protein C in a mammal by administering an amount of the vector construct, rAAV particle or pharmaceutical composition effective to increase the level of functional myosin binding protein C in the heart tissue (e.g., myocardiocytes) of a mammal. In some embodiments, the amount of the vector construct, rAAV particle or pharmaceutical composition is effective to increase the level of myosin binding protein C in heart tissue (e.g., myocardiocytes) by at least about 2-fold; and/or to restore contractile force, relative tension, calcium-activated tension, relaxation time, in engineered heart tissue in vitro or in animal tissue in vivo.

Such methods also include a method of treating HCM in a mammal, or treating or preventing any symptom thereof, comprising administering a therapeutically effective amount of the vector construct, rAAV particle or pharmaceutical composition. In one or more embodiments, such methods increase levels of cMyBP-C expression in the heart, by at least 50%, at least 60%, at least 70%, at least 80%, at least 90%, at least 95%, or at least 98% compared to the levels without treatment, or to the levels seen in healthy humans. Such methods, for example, reduce heart size, reduce cardiothoracic ratio, reduce end diastolic or end systolic left ventricular diameter, reduce anterior or posterior wall thickness, increase ejection time, increase aortic peak flow velocity or aortic flow time, and/or decrease symptoms of disease. In one or more embodiments, such methods reduce the frequency or severity of symptoms such as heart failure, arrhythmias, chest pain, shortness of breath, fatigue and dizziness.

In any of the methods described herein, the rAAV particle is delivered at a dose of about 1e12 to 6e14 vg/kg in an aqueous suspension. In any of the methods described herein, the administration of the vector construct, rAAV particle, or pharmaceutical composition may further comprise administration of prophylactic or therapeutic corticosteroid treatment, and/or may further include administration of a second therapy for treating HCM. In any of the methods herein, prior to administration of an AAV particle to a patient as described above, the prospective patient may be assessed for the presence of anti-AAV capsid antibodies or anti-AAV neutralizing antibodies that are capable of blocking cell transduction or otherwise reduce the overall efficiency of the treatment.

Other embodiments will be evident to one skilled in the art upon reading the present specification.

### BRIEF DESCRIPTION OF THE DRAWINGS

FIG. 1 depicts the organization of the elements of AAV particles comprising vectors denoted C1-C5 and A1-A6 herein.

FIG. 2 depicts the fold change of cMyBP-C protein as detected by Western blot in whole protein lysates of engineered heart tissue for vectors denoted C1-C5 and A1-A6 herein.

FIG. 3 depicts normalized force of contraction of myocytes in engineered heart tissue treated with AAV particles comprising vectors denoted C2, C3, and A2-A6 herein.

FIGS. 4A-4C depict relaxation time after contraction of myocytes in engineered heart tissue treated with AAV particles comprising vectors denoted C2, C3 and A1-A6 herein. FIG. 4A shows relative percentage of late relaxation time. FIG. 4B shows time to 20% relaxation in seconds, FIG. 4C shows time to 80% relaxation in seconds, and FIG. 4D depicts normalization of force % for Constructs A3 and A6 produced in HEK293 cells (Group 3) and insect cells (Group 4).

FIGS. 5A-5C depict DNA copy number (vector genomes), RNA copy number, and cMyBP-C protein (ug/gram of heart tissue) in mice, respectively, administered AAV particles comprising vectors denoted C1-C5 and A1-A6 herein.

FIG. 6 depicts the percentage of cardiomyocytes in heart tissue that express human cMyBP-C, from mice administered AAV particles comprising vectors denoted C3, A5 and A6 herein.

### DETAILED DESCRIPTION

Provided herein are nucleic acids or vector constructs encoding functionally active therapeutic cMyBP-C protein, AAV vector genomes and replication deficient rAAV particles comprising such vector constructs, and pharmaceutical compositions comprising such vector constructs, vector genomes and AAV particles. The compositions and methods of the invention may provide improved AAV virus production yield and/or simplified purification and/or enhanced expression of cMyBP-C protein in the heart, particularly in cells of the myocardium (cardiomyocytes). Also provided herein are methods of making the vector constructs, AAV vector genomes and replication deficient rAAV particles comprising such vector constructs. Further provided herein are methods of treating a deficiency in functional wild-type cMyBP-C, including HCM.

In another embodiment, provided are methods of producing recombinant adeno-associated virus (AAV) particles comprising any of the AAV vector constructs provided herein. The methods comprise the steps of culturing a cell that has been transfected with any of the AAV vector constructs provided herein (in association with various AAV cap and rep genes) and recovering recombinant therapeutic AAV particles from the transfected cell or supernatant of the transfected cell culture.

The cells useful for recombinant AAV production provided herein are any cell type susceptible to baculovirus infection, including insect cells such as High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5, and Ao38. In another embodiment, mammalian cells such as HEK293, HeLa, CHO, NSO, SP2/0, PER.C6, Vero, RD, BHK, HT 1080, A549, Cos-7, ARPE-19, and MRC-5 can be used.

In another embodiment, provided herein is the use of an effective amount of vector nucleic acid, vector construct, or AAV particle for the preparation of a medicament for the treatment of a subject suffering from HCM or deficiency in functional wild-type cMyBP-C protein. In one embodiment, the subject suffering from HCM is a human. In one embodiment, the medicament is administered by intravenous (IV) administration. In another embodiment, administration of the medicament results in increased levels of functional cMyBP-C in the cells of the myocardium to ameliorate HCM symptoms. In certain embodiments, the medicament is also for co-administration with a prophylactic and/or therapeutic corticosteroid for the prevention and/or treatment of any toxicity associated with administration of the AAV particle. The prophylactic or therapeutic corticosteroid treatment may comprise at least 5, 10, 15, 20, 25, 30, 35, 40, 45, 50, 55, 60, or more mg/day of the corticosteroid. In certain embodiments, the prophylactic or therapeutic corticosteroid may be administered over a continuous period of at least about 3, 4, 5, 6, 7, 8, 9, 10 weeks, or more.

In another embodiment, the hypertrophic cardiomyopathy therapy provided herein optionally further includes administration, e.g., concurrent administration, of other therapies that are used to treat HCM.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2^{nd} ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, in the context of gene delivery, the term “vector” or “gene delivery vector” may refer to a particle that functions as a gene delivery vehicle, and which comprises nucleic acid (i.e., the vector genome comprising any of the vector constructs described herein) packaged within, for example, an envelope or capsid. A gene delivery vector may be a viral gene delivery vector or a non-viral gene delivery vector. Alternatively, in some contexts, the term “vector” may be used to refer only to the vector genome or vector construct. Viral vectors suitable for use herein may be a parvovirus, an adenovirus, a retrovirus, a lentivirus or a herpes simplex virus. The parvovirus may be an adenovirus-associated virus (AAV).

As used herein, the term “AAV” is a standard abbreviation for adeno-associated virus. Adeno-associated virus is a single-stranded DNA parvovirus that grows only in cells in which certain functions are provided by a co-infecting helper virus. There are numerous serotypes of AAV that have been characterized. General information and reviews of AAV can be found in, for example, Carter, Handbook of Parvoviruses, Vol. 1, pp. 169-228 (1989); and Berns, Virology, pp. 1743-64, Raven Press, (New York) (1990); Gao et al., Meth. Mol. Biol. 807: 93-118 (2011); Ojala et al., Mol. Ther. 26(1): 304-19 (2018). However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs).

As used herein, an “AAV vector construct” refers to nucleic acids, either single-stranded or double-stranded, having at least one of (i) an AAV 5′ inverted terminal repeat (ITR) sequence and (ii) an AAV 3′ ITR, flanking a protein-coding sequence (in one embodiment, a functional therapeutic protein-encoding sequence, e.g., cMyBP-C-encoding sequence) operably linked to transcription regulatory elements (also called “expression control elements”) that are heterologous to protein-encoding sequence and/or heterologous to the AAV viral genome, i.e., one or more promoters and/or enhancers and, optionally, a polyadenylation sequence and/or optionally one or more introns. A single-stranded AAV vector refers to nucleic acids that are present in the genome of an AAV virus particle, and can be either the sense strand or the anti-sense strand of the nucleic acid sequences disclosed herein. The size of such single-stranded nucleic acids is provided in bases. A double-stranded AAV vector refers to nucleic acids that are present in the DNA of plasmids, e.g., pUC19, or genome of a double-stranded virus, e.g., baculovirus, used to express or transfer the AAV vector nucleic acids. The size of such double-stranded nucleic acids in provided in base pairs (bp).

The AAV vector constructs provided herein in single strand form are less than about 7.0 kb in length, or are less than 6.5 kb in length, or are less than 6.4 kb in length, or are less than 6.3 kb in length, or are less than 6.2 kb in length, or are less than 6.0 kb in length, or are less than 5.8 kb in length, or are less than 5.6 kb in length, or are less than 5.5 kb in length, or are less than 5.4 kb in length, or are less than 5.3 kb in length, or are less than 5.2 kb in length. The AAV vector constructs in single strand form are also at least about 4.0 kb in length. Preferably, the AAV vector constructs are also at least about 4.5 kb in length. In some embodiments, the AAV vector constructs provided herein in single strand form range from about 4.0 kb to about 5.8 kb in length.

While AAV particles have been reported in the literature having AAV genomes of >5.0 kb, in many of these cases the 5′ or 3′ ends of the encoded genes appear to be truncated (see Hirsch et al., Molec. Ther. 18: 6-8 (2010), and Ghosh et al., Biotech. Genet. Engin. Rev. 24: 165-78 (2007). It has been shown, however, that overlapping homologous recombination occurs in AAV infected cells between nucleic acids having 5′ end truncations and 3′ end truncations so that a “complete” nucleic acid encoding the large prot'in is generated, there'y reconstructing a functional, full-length gene.

Oversized AAV vectors are randomly truncated at the 5′ ends and lack a 5′ AAV ITR. Because AAV is a single-stranded DNA virus, and packages either the sense or antisense strand, the sense strand in oversized AAV vectors lacks the 5′ AAV ITR and possibly portions of the 5′ end of the target protein-coding gene, and the antisense strand in oversized AAV vectors lacks the 3′ ITR and possibly portions of the 3′ end of the target protein-coding gene. A functional transgene is produced in oversized AAV vector infected cells by annealing of the sense and antisense truncated genomes within the target cell. Thus, in certain embodiments, the AAV cMyBP-C vectors and/or viral particles comprise at least one ITR.

The term “inverted terminal repeat (ITR)” as used herein refers to the art-recognized regions found at the 5′ and 3′ termini of the AAV genome which function in cis as origins of DNA replication and as packaging signals for the viral genome. AAV ITRs, together with the AAV rep coding region, provide for efficient excision and rescue from, and integration of a nucleotide sequence interposed between two flanking ITRs into a host cell genome. Sequences of certain AAV-associated ITRs are disclosed by Yan et al., J. Virol. 79: 364-79 (2005) which is herein incorporated by reference in its entirety. ITR sequences that find use herein may be full length, wild-type AAV ITRs or fragments thereof that retain functional capability, or may be sequence variants of full-length, wild-type AAV ITRs that are capable of functioning in cis as origins of replication. AAV ITRs useful in the recombinant AAV cMyBP-C vectors of the embodiments provided herein may be derived from any known AAV serotype and, in certain embodiments, derived from the AAV2 or AAV5 serotype.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “transcription regulatory element” refers to nucleotide sequences of a gene involved in regulation of genetic transcription including a promoter, plus response elements, activator and enhancer sequences for binding of transcription factors to aid RNA polymerase binding and promote expression, and operator or silencer sequences to which repressor proteins bind to block RNA polymerase attachment and prevent expression. The term “cardiomyocyte-specific transcription regulatory element” or “cardiomyocyte-specific expression control element” refers to a regulatory element or region that produces preferred gene expression specifically in cardiomyocytes, e.g., a promoter whose activity in cardiac cells is at least 2-fold or at least 5-fold higher than in any other non-cardiac cell type. In some embodiments, the cardiomyocyte-specific promoter provides expression in cardiomyocytes at least 5-fold higher than in skeletal muscle cells. In some embodiments, the cardiomyocyte-specific promoter has an activity in cardiomyocytes at least 5-fold, at least 10-fold, at least 15-fold, at least 20-fold, at least 25-fold, or at least 50-fold higher compared to its activity in a non-cardiac cell type.

The cardiac-specific or cardiomyocyte-specific promoter is operably linked to the nucleic acid sequence encoding the cMyBP-C protein which means that the promoter is combined with the coding nucleic acid so as to enable the expression of said coding nucleic acid under the control of the promoter in cardiomyocytes when integrated into the genome of the cell or present as an extragenomic nucleic acid construct in the cell.

Transcription regulatory elements optionally include an enhancer element, intron, polyadenylation sequence, or post-transcriptional regulatory elements for increasing the expression level of the myosin binding protein. Examples include the SV40 early gene enhancer and the enhancer of the long terminal repeat (LTR) of Rous Sarcoma Virus (Gorman et al. (1982) Proc. Natl. Acad. Sci. 79:6777). The vector also optionally comprises transcription termination sequences and polyadenylation sequences for improved expression of the human and/or non-human antigen(s). Suitable transcription terminator and polyadenylation signals can, for example, be derived from SV40 (Sambrook et al (1989), Molecular Cloning: A Laboratory Manual). Preferably, a bGH polyadenylation signal is used in the vector of the invention. Any other element which is known in the art to support efficiency or specificity of expression may be added to the expression vector, such as the Woodchuck hepatitis post-transcriptional regulatory element (wPRE). To increase the cardiac or cardiomyocyte specificity, other elements can be introduced to inactivate the expression of genes in other tissues, such as sequences encoding miRNAs such as miR122 (Geisler et al., Gene Ther. 18: 199-209 (2011).

As used herein, an “intron” is broadly defined as a sequence of nucleotides that is removable by RNA splicing. “RNA splicing” means the excision of introns from a pre-mRNA to form a mature mRNA. Introns may be upstream, downstream, or within the coding region of a gene. Insertion of an intron into a nucleotide sequence can be accomplished by any method known in the art. The only limitation of where the intron is inserted is in consideration of the packaging limitations of the AAV virus particles (e.g., about 5 kb).

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

In certain embodiments, the recombinant AAV vector construct comprises (a) a nucleic acid comprising an AAV2 5′ inverted terminal repeat (ITR) (which may or may not be modified as known in the art), (b) a cardiomyocyte-specific transcription regulatory region, (c) a functional cMyBP-C protein coding region, (d) optionally, one or more introns, (e) a polyadenylation sequence, and (f) an AAV2 3′ ITR (which may or may not be modified as known in the art).

In one embodiment, the vector construct comprises a nucleic acid encoding a functionally active cMyBP-C protein. The cMyBP-C encoding sequence may be wild-type, codon optimized, or a variant. To visualize the exogenous gene expression in the heart, other optional elements can be introduced as part of the cMyBP-C encoding sequence, such as tag sequences (myc, FLAG, HA, His, and the like), or fluorochromes such as GFP, YFP, RFP.

As used herein, wild-type cardiac myosin binding protein C (MYBPC3 gene) has the following nucleic acid sequence SEQ ID NO: 1 (GenBank Accession No. NM_000256.2)

catggtgagtgcctggtgtgacgtctctcaggatgcctgagccggggaagaagccagtctcagcctttagcaagaag
ccacggtcagtggaagtggccgcaggcagccctgccgtgttcgaggccgagacagagcgggcaggagtgaaggtgcg
ctggcagcgcggaggcagtgacatcagcgccagcaacaagtacggcctggccacagagggcacacggcatacgctga
cagtgcgggaagtgggccctgccgaccagggatcttacgcagtcattgctggctcctccaaggtcaagttcgacctc
aaggtcatagaggcagagaaggcagagcccatgctggcccctgcccctgcccctgctgaggccactggagcccctgg
agaagccccggccccagccgctgagctgggagaaagtgccccaagtcccaaagggtcaagctcagcagctctcaatg
gtcctacccctggagcccccgatgaccccattggcctcttcgtgatgcggccacaggatggcgaggtgaccgtgggt
ggcagcatcaccttctcagcccgcgtggccggcgccagcctcctgaagccgcctgtggtcaagtggttcaagggcaa
atgggtggacctgagcagcaaggtgggccagcacctgcagctgcacgacagctacgaccgcgccagcaaggtctatc
tgttcgagctgcacatcaccgatgcccagcctgccttcactggcagctaccgctgtgaggtgtccaccaaggacaaa
tttgaatgctccaacttcaatctcactgtccacgaggccatgggcaccggagacctggacctcctatcagccttccg
ccgcacgagcctggctggaggtggtcggcggatcagtgatagccatgaggacactgggattctggacttcagctcac
tgctgaaaaagagagacagtttccggaccccgagggactcgaagctggaggcaccagcagaggaggacgtgtgggag
atcctacggcaggcacccccatctgagtacgagcgcatcgccttccagtacggcgtcactgacctgcgcggcatgct
aaagaggctcaagggcatgaggcgcgatgagaagaagagcacagcctttcagaagaagctggagccggcctaccagg
tgagcaaaggccacaagatccggctgaccgtggaactggctgaccatgacgctgaggtcaaatggctcaagaatggc
caggagatccagatgagcggcagcaagtacatctttgagtccatcggtgccaagcgtaccctgaccatcagccagtg
ctcattggcggacgacgcagcctaccagtgcgtggtgggtggcgagaagtgtagcacggagctctttgtgaaagagc
cccctgtgctcatcacgcgccccttggaggaccagctggtgatggtggggcagcgggtggagtttgagtgtgaagta
tcggaggagggggcgcaagtcaaatggctgaaggacggggtggagctgacccgggaggagaccttcaaataccggtt
caagaaggacgggcagagacaccacctgatcatcaacgaggccatgctggaggacgcggggcactatgcactgtgca
ctagcgggggccaggcgctgcgtgagctcattgtgcaggaaaagaagctggaggtgtaccagagcatcgcagacctg
atggtgggcgcaaaggaccaggcggtgttcaaatgtgaggtctcagatgagaatgttcggggtgtgtggctgaagaa
tgggaaggagctggtgcccgacagccgcataaaggtgtcccacatcgggcgggtccacaaactgaccattgacgacg
tcacacctgccgacgaggctgactacagctttgtgcccgagggcttcgcctgcaacctgtcagccaagctccacttc
atggaggtcaagattgacttcgtacccaggcaggaacctcccaagatccacctggactgcccaggccgcataccaga
caccattgtggttgtagctggaaataagctacgtctggacgtccctatctctggggaccctgctcccactgtgatct
ggcagaaggctatcacgcaggggaataaggccccagccaggccagccccagatgccccagaggacacaggtgacagc
gatgagtgggtgtttgacaagaagctgctgtgtgagaccgagggccgggtccgcgtggagaccaccaaggaccgcag
catcttcacggtcgagggggcagagaaggaagatgagggcgtctacacggtcacagtgaagaaccctgtgggcgagg
accaggtcaacctcacagtcaaggtcatcgacgtgccagacgcacctgcggcccccaagatcagcaacgtgggagag
gactcctgcacagtacagtgggagccgcctgcctacgatggcgggcagcccatcctgggctacatcctggagcgcaa
gaagaagaagagctaccggtggatgcagctgaacttcgacctgattcaggagctgagtcatgaagcgcggcgcatga
tcgagggcgtggtgtacgagatgcgcgtctacgcggtcaacgccatcggcatgtccaggcccagccctgcctcccag
cccttcatgcctatcggtccccccagcgaacccacccacctggcagtagaggacgtctctgacaccacggtctccct
caagtggcggcccccagagcgcgtgggagcaggaggcctggatggctacagcgtggagtactgcccagagggctgct
cagagtgggtggctgccctgcaggggctgacagagcacacatcgatactggtgaaggacctgcccacgggggcccgg
ctgcttttccgagtgcgggcacacaatatggcagggcctggagcccctgttaccaccacggagccggtgacagtgca
ggagatcctgcaacggccacggcttcagctgcccaggcacctgcgccagaccattcagaagaaggtcggggagcctg
tgaaccttctcatccctttccagggcaagccccggcctcaggtgacctggaccaaagaggggcagcccctggcaggc
gaggaggtgagcatccgcaacagccccacagacaccatcctgttcatccgggccgctcgccgcgtgcattcaggcac
ttaccaggtgacggtgcgcattgagaacatggaggacaaggccacgctggtgctgcaggttgttgacaagccaagtc
ctccccaggatctccgggtgactgacgcctggggtcttaatgtggctctggagtggaagccaccccaggatgtcggc
aacacggaactctgggggtacacagtgcagaaagccgacaagaagaccatggagtggttcaccgtcttggagcatta
ccgccgcacccactgcgtggtgccagagctcatcattggcaatggctactacttccgcgtcttcagccagaatatgg
ttggctttagtgacagagcggccaccaccaaggagcccgtctttatccccagaccaggcatcacctatgagccaccc
aactataaggccctggacttctccgaggccccaagcttcacccagcccctggtgaaccgctcggtcatcgcgggcta
cactgctatgctctgctgtgctgtccggggtagccccaagcccaagatttcctggttcaagaatggcctggacctgg
gagaagacgcccgcttccgcatgttcagcaagcagggagtgttgactctggagattagaaagccctgcccctttgac
gggggcatctatgtctgcagggccaccaacttacagggcgaggcacggtgtgagtgccgcctggaggtgcgagtgcc
tcagtgaccaggctggctcctggggatggccaggtacaaccggatgccagccccgtgccaggagcctggagggaagt
tggggaaacccctccctactgttggatgtatgtgtgacaagtgtgtctcctgtgctgcgatgggggatcagcagggc
agttgtcgggcagtcctgagtgggtgttgcacagactggtccacagggctcctgaaggaagcccctggatctttggg
gtaaaaggagggtggcctcaagaaacaatgtctggggacaggcctttctggcctgctatgtcttcccaatgtttatt
gggcaataaaagataagtgcagtcacagagaactcactcttc

As used herein, wild-type cardiac myosin binding protein C has the following amino acid sequence SEQ ID NO: 2 (GenBank Accession No. NP_000247.1)

MPEPGKKPVSAFSKKPRSVEVAAGSPAVFEAETERAGVK
VRWQRGGSDISASNKYGLATEGTRHTLTVREVGPADQG
SYAVIAGSSKVKFDLKVIEAEKAEPMLAPAPAPAEATGA
PGEAPAPAAELGESAPSPKGSSSAALNGPTPGAPDDPI
GLFVMRPQDGEVTVGGSITFSARVAGASLLKPPVVKWFK
GKWVDLSSKVGQHLQLHDSYDRASKVYLFELHITDAQP
AFTGSYRCEVSTKDKFECSNFNLTVHEAMGTGDLDLLSA
FRRTSLAGGGRRISDSHEDTGILDFSSLLKKRDSFRTP
RDSKLEAPAEEDVWEILRQAPPSEYERIAFQYGVTDLRG
MLKRLKGMRRDEKKSTAFQKKLEPAYQVSKGHKIRLTV
ELADHDAEVKWLKNGQEIQMSGSKYIFESIGAKRTLTIS
QCSLADDAAYQCVVGGEKCSTELFVKEPPVLITRPLED
QLVMVGQRVEFECEVSEEGAQVKWLKDGVELTREETFKY
RFKKDGQRHHLIINEAMLEDAGHYALCTSGGQALRELI
VQEKKLEVYQSIADLMVGAKDQAVFKCEVSDENVRGVWL
KNGKELVPDSRIKVSHIGRVHKLTIDDVTPADEADYSF
VPEGFACNLSAKLHFMEVKIDFVPRQEPPKIHLDCPGRI
PDTIVVVAGNKLRLDVPISGDPAPTVIWQKAITQGNKA
PARPAPDAPEDTGDSDEWVFDKKLLCETEGRVRVETTKD
RSIFTVEGAEKEDEGVYTVTVKNPVGEDQVNLTVKVID
VPDAPAAPKISNVGEDSCTVQWEPPAYDGGQPILGYILE
RKKKKSYRWMQLNFDLIQELSHEARRMIEGVVYEMRVY
AVNAIGMSRPSPASQPFMPIGPPSEPTHLAVEDVSDTTV
SLKWRPPERVGAGGLDGYSVEYCPEGCSEWVAALQGLT
EHTSILVKDLPTGARLLFRVRAHNMAGPGAPVTTTEPVT
VQEILQRPRLQLPRHLRQTIQKKVGEPVNLLIPFQGKP
RPQVTWTKEGQPLAGEEVSIRNSPTDTILFIRAARRVHS
GTYQVTVRIENMEDKATLVLQVVDKPSPPQDLRVTDAW
GLNVALEWKPPQDVGNTELWGYTVQKADKKTMEWFTVLE
HYRRTHCVVPELIIGNGYYFRVFSQNMVGFSDRAATTK
EPVFIPRPGITYEPPNYKALDFSEAPSFTQPLVNRSVIA
GYTAMLCCAVRGSPKPKISWFKNGLDLGEDARFRMFSK
QGVLTLEIRKPCPFDGGIYVCRATNLQGEARCECRLEVR
VPQ

The term “isolated” when used in relation to a nucleic acid molecule of the present disclosure typically refers to a nucleic acid sequence that is identified and separated from at least one contaminant nucleic acid with which it is ordinarily associated in its natural source. Isolated nucleic acid may be present in a form or setting that is different from that in which it is found in nature. Isolated nucleic acid molecules therefore are distinguished from the nucleic acid molecule as it exists in natural cells.

As used herein, the term “variant” refers to a polynucleotide (or polypeptide) having a sequence substantially similar to a reference polynucleotide (or polypeptide). Procedures for the introduction of nucleotide and amino acid changes in a polynucleotide, protein or polypeptide are known to the skilled artisan (see, e.g., Sambrook et al. (1989)). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The amino acid substitutions can be conservative or non-conservative. It is preferred that the substitutions are conservative substitutions, i.e., a substitution of an amino acid residue by an amino acid of similar polarity, which acts as a functional equivalent. Preferably, the amino acid residue used as a substitute is selected from the same group of amino acids as the amino acid residue to be substituted. For example, a hydrophobic residue can be substituted with another hydrophobic residue, or a polar residue can be substituted with another polar residue having the same charge. Functionally homologous amino acids which may be used for a conservative substitution comprise, for example, non-polar amino acids such as glycine, valine, alanine, isoleucine, leucine, methionine, proline, phenylalanine, and tryptophan. Examples of uncharged polar amino acids comprise serine, threonine, glutamine, asparagine, tyrosine and cysteine. Examples of charged polar (basic) amino acids comprise histidine, arginine and lysine. Examples of charged polar (acidic) amino acids comprise aspartic acid and glutamic acid.

Also considered as variants are proteins which differ from their naturally occurring counterparts by addition, substitution or deletion of one or more (e.g., 2, 3, 4, 5, 10, or 15) additional amino acids. Additional amino acids may be present within the amino acid sequence of the original cMyBP-C protein (i.e., as an insertion), or they may be added to one or both termini of the protein. Such insertions, substitutions or deletions can take place at any position provided they do not impair the capability of the polypeptide to fulfill the function of the naturally occurring cMyBP-C protein and/or rescue the haploinsufficiency in the treated subject. Moreover, variants of cMyBP-C proteins also comprise proteins in which, compared to the original polypeptide, one or more amino acids are lacking. Such deletions may affect any amino acid position provided that it does not impair the ability to fulfill the normal function of the cMyBP-C protein and/or rescue the haploinsufficiency.

Finally, variants of the cardiac cMyBP-C protein also refer to proteins which differ from the naturally occurring protein by structural modifications, such as modified amino acids. Modified amino acids are amino acids which have been modified either by natural processes, such as processing or post-translational modifications, or by chemical modification processes known in the art. Typical amino acid modifications comprise phosphorylation, glycosylation, acetylation, O-Linked N-acetylglucosamination, glutathionylation, acylation, branching, ADP ribosylation, crosslinking, disulfide bridge formation, formylation, hydroxylation, carboxylation, methylation, demethylation, amidation, cyclization and/or covalent or non-covalent bonding to 25hosphatidylinositol, flavine derivatives, lipoteichonic acids, fatty acids or lipids. Such modifications have been extensively described in the literature, e.g., in Proteins: Structure and Molecular Properties, T. Creighton, 2^{nd} edition, W. H. Freeman and Company, New York (1993). In a preferred embodiment of the invention, the nucleic acid sequence encodes a constitutively phosphorylated isoform of human cMyBP-C. It has been shown that these isoforms are particularly cardioprotective (Sadayappan et al. (2005), Circ Res 97:1156-1163; Sadayappan et al., 2006; Proc Natl Acad Sci USA 103:16918-16923).

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two polypeptide or nucleic acid sequences are identical over one or more sequence areas or regions they share identity within that region. An “aligned” sequence refers to multiple polynucleotide or polypeptide (amino acid) sequences, often containing corrections for missing or additional bases or amino acids (gaps) as compared to a reference sequence. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

“Percent (%) nucleic acid sequence identity or homology” is defined as the percentage of nucleotides in a candidate sequence that are identical with a reference sequence after aligning the respective sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Percent (%) amino acid sequence identity or homology” with respect to the cMyBP-C amino acid sequences identified herein is defined as the percentage of amino acid residues in a candidate sequence that are identical to the amino acid residues in a cMyBP-C polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Codon optimization” or “codon optimized” refers to changes made in the nucleotide sequence so that it is more likely to be expressed at a relatively high level compared to the non-codon optimized sequence. It does not change the amino acid for which each codon encodes.

An “AAV virion” or “AAV viral particle” or “AAV vector particle” or “AAV virus” refers to a viral particle composed of at least one AAV capsid protein and an encapsulated AAV vector construct as described herein. If the particle comprises a heterologous polynucleotide (i.e., a polynucleotide other than a wild-type AAV genome such as a transgene to be delivered to a mammalian cell), it is typically referred to as a “recombinant AAV vector particle” or simply an “AAV vector”. Production of AAV vector particles necessarily includes production of AAV vector genome, as such a vector genome is contained within an AAV vector particle. It is understood that reference to the polynucleotide AAV vector construct encapsulated within the vector particle, and replication thereof, refers to the AAV vector genome.

As used herein “therapeutic AAV virus” refers to an AAV virion, AAV viral particle, AAV vector particle, or AAV virus that comprises a heterologous polynucleotide that encodes a therapeutic protein such as the cMyBP-C described herein. An “AAV vector construct” or “AAV vector genome” as used herein refers to a vector construct comprising one or more polynucleotide encoding a protein of interest (also called transgenes) that are flanked by at least one AAV terminal repeat sequences (ITRs) and operably linked to one or more expression control elements. Such AAV vector constructs can be replicated and packaged into infectious viral particles when present in a host cell that has been transfected with a vector encoding and expressing rep and cap gene products. The term generally refers to recombinant AAV that are capable of infecting cells such that the infected cells express (e.g., by transcription and/or by translation) an element (e.g., nucleotide sequence, protein, etc.) of interest. To this extent, the therapeutically effective rAAV particles can include AAV particles having capsids or vector genomes (vgs) with different properties. For example, the therapeutically effective rAAV particles can have capsids with different post translation modifications. In other examples, the therapeutically effective AAV particles can contain vector genomes of differing sizes/lengths, plus or minus strand sequences, different flip/flop ITR configurations flip/flop, flop/flip, flip/flip, flop/flop, etc.), different number of ITRs (1, 2, 3, etc.), or truncations. For example, overlapping homologous recombination occurs in rAAV infected cells between nucleic acids having 5′ end truncations and 3′ end truncations so that a “complete” nucleic acid encoding the large protein is generated, thereby reconstructing a functional, full-length gene. In other examples, complementary nucleic acid sequences having 5′ end truncations and 3′ end truncations interact with each such that a “complete” nucleic acid is formed during second strand synthesis. The “complete” nucleic acid encodes the large protein, thereby reconstructing a functional, full-length gene. Therapeutically effective rAAV particles are also referred to as heavy capsids, full capsids, or partially full capsids. Conversely, “therapeutically ineffective” AAV virus refer to empty capsids, i.e., capsids that have unquantifiable or undetectable vector genomes, or vector genomes that are not capable of recombining into a complete functional nucleic acid.

As used herein “therapeutic protein” refers to a polypeptide that has a biological activity that replaces or compensates for the loss or reduction of activity of an endogenous protein. For example, a functional cMyBP-C protein is a therapeutic protein for HCM.

“Hypertrophic cardiomyopathy” as used herein refers to an inherited disease caused by mutations in genes encoding components of the cardiac sarcomere, such as cardiac myosin binding protein C, that is characterized, for example, by symptoms of heart failure, arrhythmias, chest pain, shortness of breath, fatigue and dizziness, increased heart size, increased cardiothoracic ratio, increased end diastolic left ventricular diameter, increased end systolic left ventricular diameter, increased ventricular (anterior or posterior or both) wall thickness, decreased ejection time, decreased aortic peak flow velocity, and/or decreased aortic flow time.

“Cardiac myosin binding protein C deficiency” or a “deficiency in functional wild-type cardiac myosin binding protein C” as used herein refers to an inherited condition caused by reduced levels of functional cMyBP-C protein, due to absence of protein, reduced production of protein or production of protein that is nonfunctional. This includes HCM.

“Therapeutically effective for hypertrophic cardiomyopathy” or “Hypertrophic cardiomyopathy therapy” as used herein refers to any therapeutic intervention of a subject having HCM that ameliorates the characteristic deficiency in functional wild-type cMyBP-C, increases cMyBP-C protein levels, e.g., in myocardium, ameliorates HCM symptoms, or reduces the frequency, duration or severity of HCM symptoms.

“Hypertrophic cardiomyopathy gene therapy” as used herein refers to any therapeutic intervention of a subject having HCM that involves the replacement or restoration or increase of cMyBP-C through the delivery of one or more nucleic acid molecules to the cells of the subject that express functional cMyBP. In certain embodiments, MYBPC3 gene therapy refers to gene therapy involving an adeno associated viral (AAV) particle comprising a vector construct that expresses human cMyBP-C. In other embodiments, the gene therapy involves transfecting a plasmid that expresses human cMyBP-C.

“Treat” or “treatment” as used herein refers to preventive or therapeutic treatment which refers to a treatment administered to a subject who exhibits signs or symptoms of pathology, i.e., HCM, for the purpose of diminishing or eliminating those signs or symptoms or ameliorating their progression, severity or duration. The signs or symptoms can be biochemical, cellular, histological, functional, subjective or objective.

“Ameliorate” as used herein refers to the action of lessening the severity of symptoms, progression, or duration of a disease.

As used herein “stably treating” or “stable treatment” refers to using a therapeutic vector construct, AAV particle or cell administered to a subject where the subject stably expresses a therapeutic protein expressed by the vector construct, AAV particle or cell. Stably expressed therapeutic protein means that the protein is expressed for a clinically significant length of time. “Clinically significant length of time” as used herein means expression at therapeutically effective levels for a length of time that has a meaningful impact on the quality of life of the subject, e.g., demonstrated by reduced signs or symptoms of disease. In certain embodiments clinically, significant length of time is expression for at least six months, for at least eight months, for at least one year, for at least two years, for at least three years, for at least four years, for at least five years, for at least six years, for at least seven years, for at least eight years, for at least nine years, for at least ten years, or for the life of the subject.

As used herein, the term “effective amount” refers to an amount sufficient to effect beneficial or desirable biological and/or clinical results.

As used herein, a “subject” refers to an animal that is the object of treatment, observation or experiment. “Animal” includes cold- and warm-blooded vertebrates and invertebrates such as fish, shellfish, reptiles, and in particular, mammals. The term “avian” as used herein includes, but is not limited to, chickens, ducks, geese, quail, turkeys and pheasants. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human, including an infant, child or juvenile human, e.g., a human age up to 2, 2-4, 2-6 or 2-12.

In general, a “pharmaceutically acceptable carrier” is one that is not toxic or unduly detrimental to cells and is preferably sterile. Exemplary pharmaceutically acceptable carriers include sterile, pyrogen-free water and sterile, pyrogen-free, saline or phosphate buffered saline. Pharmaceutically acceptable carriers include physiologically acceptable carriers. The term “pharmaceutically acceptable carrier” includes any and all solvents, dispersion media, coatings, antibacterial and antifungal agents, isotonic and absorption delaying agents, and the like that are physiologically compatible.

### Vector Constructs and AAV Vectors

The recombinant vector construct of the disclosure may be used itself as gene therapy, or may be used to produce rAAV particles by methods described herein, comprising providing to a suitable host cell the recombinant vector construct, together with Rep and Cap genes. The vector constructs described herein comprise a nucleic acid sequence that encodes a functional cMyBP-C. The recombinant vector construct may comprise a nucleic acid encoding functional human cMyBP-C operably linked to a heterologous expression control element, e.g., a promoter and/or enhancer; optionally an intron; and optionally a polyadenylation (polyA) signal. The heterologous expression control element may be a heterologous cardiomyocyte-specific transcription regulatory region, e.g., as described herein.

When used to produce rAAV particles, the recombinant vector construct may comprise (a) one or both of (i) an AAV 5′ inverted terminal repeat (ITR) sequence and (ii) an AAV 3′ ITR, (b) a heterologous cardiomyocyte-specific transcription regulatory region, and (c) a nucleic acid encoding a functional human cMyBP-C, optionally wherein the AAV ITRs are AAV2 ITRs. Preferably, the nucleic acid encoding the functional cMyBP-C is operably linked to cardiomyocyte-specific expression control elements. The vector construct may include additional expression control elements, for example: a promoter and/or enhancer; an intron; optionally an exon or fragment thereof; and a polyadenylation (polyA) signal. Such elements are further described herein. In certain embodiments, the recombinant AAV vector construct comprises a nucleic acid comprising (a) an AAV2 5′ inverted terminal repeat (ITR) (which may or may not be modified as known in the art), (b) a cardiomyocyte-specific transcription regulatory region, a functional MYBPC3 protein coding region, (c) one or more introns including fragments of longer introns, (d) optionally an exon or fragment thereof, I a polyadenylation sequence, and (f) an AAV2 3′ ITR (which may or may not be modified as known in the art).

Preferably, the rAAV particles also comprise an AAV capsid with cardiac tropism, optionally an AAV9 type capsid. Example capsids with cardiac tropism include AAV1, 6, 7 and 9.

Other embodiments provided herein are directed to vector constructs encoding a functional cMyBP-C polypeptide, wherein the constructs comprise one or more of the individual elements of the above described constructs and combinations thereof, in one or more different orientation(s). Another embodiment provided herein is directed to the above-described constructs in an opposite orientation.

The AAV vector constructs provided herein in single strand form range from about 4.5 kb to about 6.5 kb in length, or from about 4.5 kb to about 5.5 kb in length, or from about 4 kb to about 5.5 kb in length, or range from about 4.8 kb to about 5.2 k in length, or 4.8 kb to 5.1 kb in length, or range from about 4.9 kb to about 5.5 kb in length, or about 4.8 kb to about 6.0 kb in length, or about 5.0 kb to 6.2 kb in length or about 5.1 kb to about 6.3 kb in length, or about 5.2 kb to about 6.4 kb in length, or about 5.5 kb to about 6.5 kb in length, or range from about 4.0 kb to about 5.0 kb in length, or range from about 4 to about 4.5 kb in length, or range from about 4.5 kb to about 5 kb in length.

When AAV vectors are produced from oversized recombinant vector constructs, they may lack a portion of the 5′ or 3′ ends of the recombinant vector construct. Because AAV is a single-stranded DNA virus, and packages either the sense or antisense strand, the sense strand in oversized AAV vectors lacks the 5′ AAV ITR and possibly portions of the 5′ end of the target protein-coding gene, and the antisense strand in oversized AAV vectors lacks the 3′ ITR and possibly portions of the 3′ end of the target protein-coding gene. A functional transgene is produced in oversized AAV vector infected cells by annealing of the sense and antisense truncated genomes within the target cell. Thus, in certain embodiments, the rAAV particles of the invention may comprise recombinant vector constructs that comprise at least one ITR, and a substantial portion of a nucleotide sequence encoding a functional cMyBP-C, such as a fragment of SEQ ID NO: 1 or 42-45 that is greater than 50%, 60%, 70%, 80%, or 90% of the length of the nucleotide sequence. For example, the recombinant vector construct may comprise at least one ITR, a cardiomyocyte-specific transcription regulatory region, and a substantial portion of a nucleotide sequence encoding a functional cMyBP-C.

Generation of the vector constructs can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

The vector constructs can incorporate sequences from the genome of any known organism. The sequences can be incorporated in their native form or can be modified in any way to obtain a desired activity. For example, the sequences can comprise insertions, deletions or substitutions.

AAV vector constructs can be replicated and packaged into infectious AAV particles, preferably replication deficient AAV particles, when present in a host cell that has been transfected with a polynucleotide encoding and expressing rep and cap gene product

### Transcription Regulatory Elements or Region

### Promoters and Enhancers.

In one or more embodiments, the nucleic acid sequence encoding cMyBP-C is operably linked to one or more heterologous expression control elements. Preferably, the expression control element is a cardiomyocyte-specific expression control element. Examples of cardiomyocyte-specific control elements include, but are not limited to human cardiac troponin T (hTNNT2) promoter or fragments or variants thereof. Other promoters with activity in cardiomyocytes include fragments or variants of any of: muscle creatine kinase (MCK) promoter, cytomegalovirus enhancer+myosin light chain 2 promoter (CMV-MLC2, or CMV-MLC1.5, CMV-MLC260), a phosphoglycerate kinase (PGK) promoter, a sarcomere-specific promoters, alpha myosin heavy chain promoter, myosin light chain 2v promoter, alpha myosin heavy chain promoter, alpha-cardiac actin promoter, alpha-tropomyosin promoter, cardiac troponin C promoter, cardiac troponin I promoter, cardiac myosin-binding protein C promoter, and/or sarco/endoplasmic reticulum Ca2+” ATPase (SERCA) promoter (e.g., iso-form 2 of this promoter (SERCA2)), and/or a striated muscle promoter, such as the desmin promoter. Enhancers derived from cardiomyocyte-specific transcriptional factor binding sites are also contemplated.

Examples of fragments or variants of hTNNT2 promoter include a cardiomyocyte-specific promoter sequence comprising a nucleic acid sequence at least 90% identical to SEQ ID NO: 47. In some embodiments, the cardiomyocyte-specific transcription regulatory region comprises a cardiomyocyte-specific promoter sequence comprising a nucleic acid sequence at least 80% identical to SEQ ID NO: 52. In some embodiments, the cardiomyocyte-specific transcription regulatory region comprises a cardiomyocyte-specific promoter sequence comprising a nucleic acid sequence at least 80% identical to SEQ ID NO: 51. In some embodiments, the cardiomyocyte-specific transcription regulatory region comprises a cardiomyocyte-specific promoter sequence comprising a nucleic acid sequence at least 80% identical to SEQ ID NO: 50. In some embodiments, the cardiomyocyte-specific transcription regulatory region comprises a cardiomyocyte-specific promoter sequence comprising a nucleic acid sequence at least 80% identical to SEQ ID NO: 49. In some embodiments, the cardiomyocyte-specific transcription regulatory region comprises a cardiomyocyte-specific promoter sequence comprising a nucleic acid sequence at least, or more than, 80%, 85%, 90%, 95%, 97%, 98% or 99% identical to any one of SEQ ID Nos: 49-52 (over the length of the SEQ ID NO). In any of the embodiments described herein, the cardiomyocyte-specific promoter optionally excludes any one of SEQ ID NO: 1 to 85 of U.S. Pat. Pub. No. 2021/0252165. In some embodiments, the cardiomyocyte-specific transcription regulatory region also comprises an intron that enhances expression of the cMyBP-C protein, and optionally an exon or fragment thereof, 5′ to the cMyBP-C coding sequence. For example, the vector construct and AAV particle comprise, in 5′ to 3′ orientation, a cardiomyocyte-specific promoter comprising a nucleotide sequence at least 80% identical to SEQ ID NO: 47, an intron nucleotide sequence at least 70% identical to SEQ ID NO: 53, and a nucleotide sequence encoding cMyBP-C.

In other embodiments, the cardiomyocyte-specific promoter comprises (a) a nucleic acid sequence at least 80% identical to any of (i) SEQ ID NO: 49 or a fragment thereof, (ii) SEQ ID NO: 50 or a fragment thereof, or (iii) SEQ ID NO: 51 or a fragment thereof and (b) an intron nucleotide sequence at least 60%, 65%, 70%, 75%, 80%, 85%, 90%, 95%, 97%, 98% or 99% identical to SEQ ID NO: 53. In alternative embodiments, the intron comprises a nucleotide sequence at least 70%, 75%, 80%, 85%, 90%, 95%, 97%, 98% or 99% identical to SEQ ID NO: 58. Other exemplary introns are SEQ ID NO: 53-58.

In some embodiments, the vector construct comprises (a) a nucleic acid sequence at least 90% identical to any of (i) SEQ ID NO: 49 or a fragment thereof, (ii) SEQ ID NO: 50 or a fragment thereof, or (iii) SEQ ID NO: 51 or a fragment thereof and (b) an intron comprising a nucleotide sequence at least 70%, 75%, 80%, 85%, 90%, 95%, 97%, 98% or 99% identical to SEQ ID NO: 53. In alternative embodiments, the intron comprises a nucleotide sequence at least 70%, 75%, 80%, 85%, 90%, 95%, 97%, 98% or 99% identical to SEQ ID NO: 58. Other exemplary introns are SEQ ID NO: 53-58.

In some embodiments, the cardiomyocyte-specific transcription regulatory region may further comprise (in addition to the fragment or variant of hTNNT2 promoter and globin intron) an exon sequence or fragment thereof, e.g., a globin intron adjacent to the 3′ end of a fragment of beta globin exon 3 (SEQ ID NO: 54). The combination of the intron and exon fragment is, for example, SEQ ID NO: 55. In some example embodiments, the cardiomyocyte-specific transcription regulatory region comprises SEQ ID NO: 56.

In some embodiments, the fragment or variant of the hTNNT2 promoter is more than 420 and less than 544 nucleotides in length and comprises a nucleic acid sequence at least 90% identical to SEQ ID NO: 47. In any of the embodiments described herein, the cardiomyocyte-specific promoter optionally excludes any one of SEQ ID NO: 1 to 85 of U.S. Pat. Pub. No. 2021/0252165.

In some embodiments, the cardiomyocyte-specific promoter sequence comprises a nucleic acid sequence at least, or more than, 80%, 85%, 90%, 95%, 97%, 98% or 99% identical to any of (i) SEQ ID NO: 49 or a fragment thereof, (ii) SEQ ID NO: 50 or a fragment thereof, (iii) SEQ ID NO: 51 or a fragment thereof, or (iv) SEQ ID NO: 52 or a fragment thereof. For example, the cardiomyocyte-specific promoter sequence comprises a nucleic acid sequence at least, or more than, 95%, 97%, 98% or 99% identical to any of (i) SEQ ID NO: 49 or a fragment thereof, (ii) SEQ ID NO: 50 or a fragment thereof, or (iii) SEQ ID NO: 51 or a fragment thereof. In an example embodiment, the sequence of the cardiomyocyte-specific promoter comprises a nucleotide sequence at least 96%, 97%, 98%, or 99% identical to SEQ ID NO: 51. In some example embodiments, the sequence of the hTNNT promoter comprises at least nucleotides 1-106 and 507-532 of SEQ ID NO: 51, or at least nucleotides 507-532 of SEQ ID NO: 51, or at least nucleotides 521-532 of SEQ ID NO: 51.

Various promoters can be operably linked with a nucleic acid comprising the coding region of the protein of interest, human cardiac myosin binding protein C, in the vector constructs disclosed herein. In some embodiments, the promoter can drive the expression of the protein of interest in a cell infected with a virus derived from the viral vector, such as a target cell. The promoter can be naturally occurring or non-naturally occurring. In some embodiments the promoter is a synthetic promoter. In one embodiment the synthetic promoter comprises sequences that do not exist in nature and which are designed to regulate the activity of an operably linked gene. In another embodiment the synthetic promoter comprises fragments of natural promoters to form new stretches of DNA sequence that do not exist in nature. Synthetic promoters are typically comprised of regulatory elements, promoters, enhancers, introns, splice donors and acceptors that are designed to produce enhanced tissue specific expression. Examples of promoters include, but are not limited to, viral promoters, plant promoters and mammalian promoters. In another embodiment the promoter is a cardiomyocyte specific promoter.

In some embodiments, the promoter comprises the human cardiac troponin T (hTNNT2) promoter. The portion of the hTNNT2 promoter can comprise a nucleic acid sequence having at least about 90%, at least about 95%, at least about 96%, at least about 97%, at least about 98%, at least about 99%, or more, sequence identity to any of SEQ ID Nos: 49-51. In some embodiments, the promoter is at least about, or more than, 95% identical to any of SEQ ID Nos: 49-51.

In some embodiments, the promoter constructs comprise one or more of additional individual enhancer elements, in one or more different orientation(s).

In some embodiments, the promoter is operably linked with a polynucleotide encoding one or more proteins of interest. In some embodiments, the promoter is operably linked with a polynucleotide encoding the cMyBP-C protein.

The size of the promoter can vary. Because of the limited packaging capacity of AAV, it is preferred to use a promoter that is small in size, but at the same time allows high level production of the protein(s) of interest in host cells. For example, in some embodiments the promoter is at most about 1.5 kb, at most about 1.4 kb, at most about 1.35 kb, at most about 1.3 kb, at most about 1.25 kb, at most about 1.2 kb, at most about 1.15 kb, at most about 1.1 kb, at most about 1.05 kb, at most about 1 kb, at most about 800 base pairs, at most about 600 base pairs, at most about 400 base pairs, at most about 200 base pairs, or at most about 100 base pairs.

### Other Regulatory Elements.

Various additional regulatory elements can be used in the vector constructs, for example enhancers to further increase expression level of the protein of interest in a host cell, a polyadenylation signal, a ribosome binding sequence, and/or a consensus splice acceptor or splice donor site. In some embodiments, the regulatory element can facilitate maintenance of the recombinant DNA molecule extrachromosomally in a host cell and/or improve vector potency (e.g., scaffold/matrix attachment regions (S/MARs)). Such regulatory elements are well known in the art.

The vectors constructs disclosed herein may include regulatory elements such as a transcription initiation region and/or a transcriptional termination region. Examples of a transcription termination region include, but are not limited to, polyadenylation signal sequences. Examples of polyadenylation signal sequences include, but are not limited to, mini polyA, human growth hormone (hGH) poly(A), bovine growth hormone (bGH) poly(A), SV40 late poly(A), rabbit beta-globin (rBG) poly(A), thymidine kinase (TK) poly(A) sequences, Proudfoot polyA, and any variants thereof. In some embodiments, the transcriptional termination region is located downstream of the posttranscriptional regulatory element. In some embodiments, the transcriptional termination region is a polyadenylation signal sequence. In some embodiments, the transcriptional termination region is a mini polyA (e.g., SEQ ID NO: 64), a bGH polyA (e.g., any of SEQ ID Nos: 59-61), a hGH polyA (e.g., SEQ ID NO: 62), a SV40 polyA (e.g., SEQ ID NO: 53), a Proudfoot synthetic polyA (e.g., SEQ ID NO: 65) or a rabbit beta-globin polyA (e.g., SEQ ID NO: 66) sequence or a fragment thereof about 40 to 200 nucleotides in length.

In some embodiments, the vector construct comprises a polyadenylation signal, optionally a bovine growth hormone (bGH) polyA signal (e.g., SEQ ID Nos: 59-61) or a human growth hormone (hGH) polyA signal (e.g., SEQ ID NO: 62) or fragment thereof.

The polyA signal may be about 150 to about 250 nucleotides in length, about 160 to about 240 nucleotides in length, about 170 to about 230 nucleotides in length, about 180 to about 220 nucleotides in length, or about 200 to about 210 nucleotides in length.

In some embodiments, the vector constructs can include additional transcription and translation initiation sequences, and/or additional transcription and translation terminator sequences, which are known in the art.

### Protein of Interest and Nucleic Acids Encoding the Protein of Interest.

As used herein, a “protein of interest” is any functional cMyBP-C protein, including naturally-occurring and non-naturally occurring variants thereof. In some embodiments, a polynucleotide encoding one or more cMy-BP-C proteins of interest can be inserted into the viral vectors disclosed herein, wherein the polynucleotide is operably linked with the promoter. In some instances, the promoter can drive the expression of the protein(s) of interest in a host cell (e.g., human myocardium).

In one or more embodiments, the functional cMyBP-C comprises an amino acid sequence at least 90%, 95% or 98% identical to SEQ ID NO: 2 (a human cardiac myosin binding protein C). The present disclosure also provides an isolated nucleic acid molecule which encodes such functional wild-type cMY-BP-C protein. The nucleotide sequence may be homologous to the wild-type nucleotide sequence of SEQ ID NO: 1. In certain embodiments, the nucleic acid molecule has at least 75%, at least 80%, at least 85%, at least 90%, at least 95% homology, or at least 98% homology to the nucleotide sequence of SEQ ID NO: 1, or at least 100, 200, 300, 400 or 500 consecutive nucleotides of SEQ ID NO: 1 or 42-43. In example embodiments, the nucleotide sequence encoding the functional cardiac myosin binding protein C is codon optimized or a variant, and may be at least 85%, 90%, 95%, 97%, 98% or 99% identical any of SEQ ID Nos: 44-46.

In certain embodiments, the nucleic acid molecule has at least 75%, at least 80%, at least 85%, at least 90%, at least 95% homology, or at least 98% homology to the nucleotide sequence of SEQ ID Nos: 1 or 42-46, or at least 100, 200, 300, 400 or 500 consecutive nucleotides of SEQ ID NO: 1 or 42-46.

In example embodiments, the nucleic acid sequence encoding the functional cMyBP-C is a wild-type MYBPC3 sequence, of which SEQ ID NO: 1 is one example, or is codon optimized, or is a variant. The vector constructs described herein may comprise a nucleotide sequence that differs from wild type nucleotide sequence but still encodes a functional cMyBP-C amino acid sequence at least 90%, 95% or 98% identical to SEQ ID NO: 2. According to this aspect, the nucleotide sequence may comprise a portion having at least 80%, 85%, 90% or 95% homology to at least 100 consecutive bases of SEQ ID NO: 1 or 42-46, as long as the nucleotide sequence encodes functional human cMyBP-C protein at least 90%, 95% or 98% identical to SEQ ID NO: 2. In example embodiments, the nucleotide sequence may comprise a portion having at least 90% homology to at least 100, 200, 300, 400, or 500 consecutive bases of SEQ ID NO: 1, as long as the nucleotide sequence encodes functional human cMyBP-C protein at least 90% identical to SEQ ID NO: 2. In example embodiments, the nucleotide sequence has substantial homology to the nucleotide sequence of SEQ ID NO: 1 or 42-46 and encodes functional cMyBP-C. The term substantial homology can be further defined with reference to a percent (%) homology, e.g., at least 80%, 85%, 90% or 95% homologous. This is discussed in further detail elsewhere herein.

In example embodiments, the nucleotide sequence of the gene of interest is codon optimized, preferably codon optimized for more efficient expression in humans, or for more efficient expression in a target organ, target tissue and/or target cells of humans. Target organs, tissues or cells include heart tissue and/or cardiomyocytes. The adaptiveness of a nucleotide sequence encoding a gene therapy product to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al., Gene. 1997, 199:293-301; zur Megede et al., Journal of Virology, 2000, 74: 2628-2635). In certain embodiments, a gene of interest has a CAI of at least 0.75, 0.80, 0.85, 0.90, 0.95, or 0.99.

Codon optimization can be performed, for example, using the DNA2.0 codon optimization algorithm, see Villalobos et al., “Gene Designer: a synthetic biology tool for constructing artificial DNA segments,” BMC Bioinformatics, vol. 7, article no: 285 (2006) or Operon/Eurofins Genomics codon optimization software or other codon optimization tools, e.g., Grote et al., “Jcat: a novel tool to adapt codon usage of a target gene to its potential expression host,” Nucleic Acids Res. 33:W526-31 (2005).

In addition, or alternatively to codon optimization, the nucleotide sequence of the gene of interest can be adjusted to reduce CpG di-nucleotide content and optionally remove any extra ORF in the sense and anti-sense direction. CpG di-nucleotide content has been shown to activate TLR9 in dendritic cells leading to potential immune activation and CTL responses. Reducing CpG content may reduce liver inflammation and ALT. In some embodiments, the nucleotide sequence of the gene of interest has a CpG di-nucleotide content of less than 25, less than 20, less than 15, or less than 10. In another embodiment, the nucleotide sequence of the gene of interest has a GC content of less than 65%, less than 60%, or less than 55%.

Generally, codon optimization or CpG reduction does not change the amino acid for which each codon encodes. It simply changes the nucleotide sequence so that it is more likely to be expressed at a relatively high level compared to the non-optimized sequence.

As described herein, the nucleotide sequence encoding the cMyBP-C protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal). As another non-limiting example for the modification, one or more of the splice donors and/or splice acceptors in the nucleotide sequence of the protein of interest is modified to reduce the potential for extraneous splicing. As another non-limiting example for the modification, one or more introns can be inserted within or adjacent to the nucleotide sequence of the protein of interest to optimize AAV vector packaging and enhance expression.

The nucleic acid molecule encodes a functional cMyBP-C protein at least 90% identical to SEQ ID NO: 2 wild type amino acid sequence. If the nucleic acid encodes a protein comprising a sequence having changes to any of the wild-type amino acids, the protein should still be a functional protein. A skilled person will appreciate that minor changes can be made to some of the amino acids of the protein without adversely affecting the function of the protein.

In certain embodiments, the nucleic acid molecule, when expressed in a suitable system (e.g., a host cell), produces a functional cMyBP-C protein and at a relatively high level. Since the cMyBP-C that is produced is functional, it will have a conformation which is the same as at least a portion of the wild type cMyBP-C. In certain embodiments, a functional cMyBP-C protein produced as described herein effectively treats a subject suffering from deficiency in wild-type cMyBP-C protein and/or HCM.

It would be well within the capabilities of a skilled person to produce a nucleic acid molecule provided herein. This could be done, for example, using chemical synthesis of a given sequence. Further, suitable methods would be apparent to those skilled in the art for determining whether a nucleic acid described herein expresses a functional protein. For example, one suitable in vitro method involves inserting the nucleic acid into a vector, such as an AAV vector, transducing host cells, such as 293T or HeLa cells, with the vector, and assaying for cMyBP-C. Alternatively, a suitable in vivo method involves transducing a vector containing the nucleic acid into HCM mice and assaying for functional cMyBP-C.

### Introns

In some embodiments, the vector comprises one or more introns. The introns may facilitate processing of the RNA transcript in mammalian host cells, increase expression of the protein of interest and/or optimize packaging of the vector into AAV particles. Non-limiting examples of such an intron are a human beta globin intron, a human immunoglobulin G (IgG) intron or a native cMyBP-C intron. In some embodiments, the intron is a synthetic intron.

In some embodiments, the vector construct and/or AAV particle comprise a cardiomyocyte-specific promoter and one or more additional heterologous expression control elements, such as an intron that enhances expression of the cMyBP-C protein. For example, the vector construct and/or AAV particle comprise any of the cardiomyocyte-specific promoters as described above, and optionally an intron nucleotide sequence located 5′ to the nucleotide sequence encoding cMyBP-C. In further examples, the vector construct and/or AAV particle comprise any of the cardiomyocyte-specific promoters as described above, and optionally an intron nucleotide sequence located within the nucleotide sequence encoding cMyBP-C, for example, between any of the exons. In some embodiments, the intron sequence is located between exon 2 and 3. In some embodiments, the intron sequence is located at a position within the nucleic acid encoding cMyBP-C that corresponds to position 293 of SEQ ID NO: 1 or 42-46.

In one or more embodiments, the intron comprises a nucleotide sequence at least 60%, 65%, 70%, 75%, 80% or 85% or 90% or 95% identical to SEQ ID NO: 53, and the intron may be about 50 to about 150 nucleotides in length, or about 100 to about 135 nucleotides in length. In example embodiments, the intron comprises SEQ ID NO: 53 or a fragment thereof that is that is about 50-150 nucleotides, 75-145 nucleotides, 100-135 nucleotides, or 120-135 nucleotides of SEQ ID NO: 53 or a variant of said fragment that is at least 80%, 85%, 90%, or 95% identical to said fragment. In some embodiments, the intron can comprise a nucleotide sequence at least 70%, 75%, 80%, 85%, 90%, 95%, 97%, 98% or 99% identical to SEQ ID NO: 53.

In one or more embodiments, the intron comprises a nucleotide sequence at least 60%, 65%, 70%, 75%, 80% or 85% or 90% or 95% identical to SEQ ID NO: 58, and the intron may be about 50 to about 150 nucleotides in length, or about 100 to about 135 nucleotides in length. In example embodiments, the intron comprises SEQ ID NO: 58 or a fragment thereof that is that is about 50-150 nucleotides, 75-145 nucleotides, 100-135 nucleotides, or 120-135 nucleotides of SEQ ID NO: 58 or a variant of said fragment that is at least 80%, 85%, 90%, or 95% identical to said fragment. In some embodiments, the intron can comprise a nucleotide sequence at least 70%, 75%, 80%, 85%, 90%, 95%, 97%, 98% or 99% identical to SEQ ID NO: 58.

Other exemplary introns comprise a nucleotide sequence at least 60%, 65%, 70%, 75%, 80% or 85% or 90% or 95% identical to any one of SEQ ID NO: 53-58.

In some embodiments, the vector constructs may further comprise an exon sequence or fragment thereof; preferably adjacent to the 5′ or 3′ end of an intron sequence. In an example embodiment, the vector construct comprises a globin intron adjacent to an exon comprising a nucleotide sequence at least 80% or 85% or 90% or 95% identical to SEQ ID NO: 54. In a further example embodiment, the vector construct comprises a globin intron adjacent to an exon sequence comprising a nucleotide sequence at least 80% or 85% or 90% or 95% identical to SEQ ID NO: 53. In an example embodiment, the vector construct comprises a globin intron adjacent to an HbB exon sequence comprising a nucleotide sequence at least 80% or 85% or 90% identical to SEQ ID NO: 54.

The location and size of the intron in the vector can vary. In some embodiments, the intron is located between the promoter and the sequence encoding the protein of interest. In some embodiments, the intron is located downstream of the sequence encoding the protein of interest. In some embodiments, the intron is located within the promoter. In some embodiments, the intron includes an enhancer element. In some embodiments, the intron is located within the sequence encoding the protein of interest, preferably between exons of the sequence encoding the protein of interest. In some embodiments, the intron may comprise all or a portion of a naturally occurring intron within the sequence encoding the protein of interest. In some embodiments, the intron is a globin intron. In some embodiments, the intron is a chimeric intron and comprises a fragment of a human IgG intron.

Inclusion of an intron element may enhance expression compared with expression in the absence of the intron element (see e.g., Kurachi et al., J. Biol. Chem. 270(10): 5276-81 (1995). AAV vectors typically accept inserts of DNA having a defined size range which is generally about 4 kb to about 5.4 kb, or slightly more. However, there is no minimum size for packaging and small vector genomes package very efficiently. Introns and intron fragments fulfill this requirement while also enhancing expression. Thus, the present disclosure is not limited to the inclusion of cMyBP-C intron sequences in the AAV vector and include other introns or other DNA sequences in place of portions of a cMyBP-C intron. Additionally, other 5′ and 3′ untranslated regions of nucleic acid may be used in place of those recited for human cMyBP-C.

In some embodiments, the vector construct comprises a nucleotide sequence at least 97%, 98% or 99% identical to any of SEQ ID NO: 3-41 or 92-169. In other embodiments, the vector construct comprises a nucleotide sequence at least 97%, 98% or 99% identical to a nucleotide sequence that is complementary to or is a negative (−) strand of any of SEQ ID NO: 3-41 or 92-169.

In some embodiments, the vector construct comprises a nucleotide sequence at least 97%, 98% or 99% identical to any of SEQ ID NO: 3, 6, 9, 12, 15, 18, 21, 24, 27, 30, 33, 36, or 39. In other embodiments, the vector construct comprises a nucleotide sequence at least 97%, 98% or 99% identical to a nucleotide sequence that is complementary to or is a negative (−) strand of any of SEQ ID NO: 3, 6, 9, 12, 15, 18, 21, 24, 27, 30, 33, 36, or 39.

In some embodiments, the vector construct comprises a nucleotide sequence at least 97%, 98% or 99% identical to any of SEQ ID NO: 4, 7, 10, 13, 16, 19, 22, 25, 28, 31, 34, 37, or 40. In other embodiments, the vector construct comprises a nucleotide sequence at least 97%, 98% or 99% identical to a nucleotide sequence that is complementary to or is a negative (−) strand of any of SEQ ID NO: 4, 7, 10, 13, 16, 19, 22, 25, 28, 31, 34, 37, or 40.

In some embodiments, the vector construct comprises a nucleotide sequence at least 97%, 98% or 99% identical to any of SEQ ID NO: 5, 8, 11, 14, 17, 20, 23, 26, 29, 32, 35, 38, or 41. In other embodiments, the vector construct comprises a nucleotide sequence at least 97%, 98% or 99% identical to a nucleotide sequence that is complementary to or is a negative (−) strand of any of 5, 8, 11, 14, 17, 20, 23, 26, 29, 32, 35, 38, or 41.

Example embodiments include the following: Construct C1 is 4950 bp in length (SEQ ID NO: 29) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (130 bp) (SEQ ID NO: 71).

In some embodiments, Construct C1 is 4980 bp in length (SEQ ID NO: 28) and comprises the following elements in 5′ to 3′: 5′ AAV2-ITR (145 bp) (SEQ ID NO: 68), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (145 bp) (SEQ ID NO: 72).

In some embodiments, Construct C1 is 4950 bp in length (SEQ ID NO: 92) and comprises the following elements in 5′ to 3′: 5′ AAV2-ITR (130 bp) (SEQ ID NO: 69), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (130 bp) (SEQ ID NO: 73).

In some embodiments, Construct C1 is 4980 bp in length (SEQ ID NO: 93) and comprises the following elements in 5′ to 3′: 5′ AAV2-ITR (145 bp) (SEQ ID NO: 70), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (145 bp) (SEQ ID NO: 74).

In some embodiments, Construct C1 is 4950 bp in length (SEQ ID NO: 94) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (130 bp) (SEQ ID NO: 73).

In some embodiments, Construct C1 is 4980 bp in length (SEQ ID NO: 95) and comprises the following elements in 5′ to 3′: 5′ AAV2-ITR (145 bp) (SEQ ID NO: 68), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (145 bp) (SEQ ID NO: 74).

In some embodiments, Construct C1 is 4950 bp in length (SEQ ID NO: 96) and comprises the following elements in 5′ to 3′: 5′ AAV2-ITR (130 bp) (SEQ ID NO: 69), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (130 bp) (SEQ ID NO: 69).

In some embodiments, Construct C1 is 4980 bp in length (SEQ ID NO: 97) and comprises the following elements in 5′ to 3′: 5′ AAV2-ITR (145 bp) (SEQ ID NO: 70), hTNNT2 promoter (544 bp) (SEQ ID NO: 52), chimeric intron (133 bp) (SEQ ID NO: 58), codon optimized hMYBPC3 (SEQ ID NO: 44), mini poly A (57 bp) (SEQ ID NO: 64) and 3′ AAV2 ITR (145 bp) (SEQ ID NO: 72).

In some embodiments, Construct C1 is 4640 bp in length (SEQ ID NO: 27) and comprises the following elements in 5′ to 3′: hTNNT2 promoter (544 bp) (SEQ ID NO: 52), chimeric intron (133 bp) (SEQ ID NO: 58), codon optimized hMYBPC3 (SEQ ID NO: 44), and mini poly A (57 bp) (SEQ ID NO: 64). In further embodiments, Construct C1 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C2 is 4801 bp in length (SEQ ID NO: 32) and comprises the following elements in 5′ to 3′: 5′ AAV2-ITR (130 bp) (SEQ ID NO: 67), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (130 bp) (SEQ ID NO: 71).

In some embodiments, Construct C2 is 4831 bp in length (SEQ ID NO: 31) and comprises the following elements in 5′ to 3′: 5′ AAV2-ITR (145 bp) (SEQ ID NO: 68), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (145 bp) (SEQ ID NO: 72).

In some embodiments, Construct C2 is 4801 bp in length (SEQ ID NO: 98) and comprises the following elements in 5′ to 3′: 5′ AAV2-ITR (130 bp) (SEQ ID NO: 69), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (130 bp) (SEQ ID NO: 73).

In some embodiments, Construct C2 is 4831 bp in length (SEQ ID NO: 99) and comprises the following elements in 5′ to 3′: 5′ AAV2-ITR (145 bp) (SEQ ID NO: 70), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (145 bp) (SEQ ID NO: 74).

In some embodiments, Construct C2 is 4801 bp in length (SEQ ID NO: 100) and comprises the following elements in 5′ to 3′: 5′ AAV2-ITR (130 bp) (SEQ ID NO: 67), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (130 bp) (SEQ ID NO: 73).

In some embodiments, Construct C2 is 4831 bp in length (SEQ ID NO: 101) and comprises the following elements in 5′ to 3′: 5′ AAV2-ITR (145 bp) (SEQ ID NO: 68), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (145 bp) (SEQ ID NO: 74).

In some embodiments, Construct C2 is 4801 bp in length (SEQ ID NO: 102) and comprises the following elements in 5′ to 3′: 5′ AAV2-ITR (130 bp) (SEQ ID NO: 69), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (130 bp) (SEQ ID NO: 71).

In some embodiments, Construct C2 is 4831 bp in length (SEQ ID NO: 103) and comprises the following elements in 5′ to 3′: 5′ AAV2-ITR (145 bp) (SEQ ID NO: 70), hTNNT2 promoter (544 bp) (SEQ ID NO: 52), codon optimized hMYBPC3 (SEQ ID NO: 44), mini poly A (57 bp) (SEQ ID NO: 64) and 3′ AAV2 ITR (145 bp) (SEQ ID NO: 72).

In some embodiments, Construct C2 is 4491 bp in length (SEQ ID NO: 30) and comprises the following elements in 5′ to 3′: hTNNT2 promoter (544 bp) (SEQ ID NO: 52), codon optimized hMYBPC3 (SEQ ID NO: 44), and mini poly A (57 bp) (SEQ ID NO: 64). In further embodiments, Construct C2 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C3 is 4801 bp in length (SEQ ID NO: 35) and comprises the following elements in 5′ to 3′: 5′ AAV2-ITR (130 bp) (SEQ ID NO: 67), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 71).

In some embodiments, Construct C3 is 4831 bp in length (SEQ ID NO: 34) and comprises the following elements in 5′ to 3′: 5′ AAV2-ITR (145 bp) (SEQ ID NO: 68), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 72).

In some embodiments, Construct C3 is 4801 bp in length (SEQ ID NO: 104) and comprises the following elements in 5′ to 3′: 5′ AAV2-ITR (130 bp) (SEQ ID NO: 69), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 73).

In some embodiments, Construct C3 is 4831 bp in length (SEQ ID NO: 105) and comprises the following elements in 5′ to 3′: 5′ AAV2-ITR (145 bp) (SEQ ID NO: 70), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 74).

In some embodiments, Construct C3 is 4801 bp in length (SEQ ID NO: 106) and comprises the following elements in 5′ to 3′: 5′ AAV2-ITR (130 bp) (SEQ ID NO: 67), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 73).

In some embodiments, Construct C3 is 4831 bp in length (SEQ ID NO: 107) and comprises the following elements in 5′ to 3′: 5′ AAV2-ITR (145 bp) (SEQ ID NO: 68), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 74).

In some embodiments, Construct C3 is 4801 bp in length (SEQ ID NO: 108) and comprises the following elements in 5′ to 3′: 5′ AAV2-ITR (130 bp) (SEQ ID NO: 69), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 71).

In some embodiments, Construct C3 is 4831 bp in length (SEQ ID NO: 109) and comprises the following elements in 5′ to 3′: 5′ AAV2-ITR (145 bp) (SEQ ID NO: 70), hTNNT2 promoter (544 bp) (SEQ ID NO: 52), wild type hMYBPC3 (SEQ ID NO: 43), mini poly A (57 bp) (SEQ ID NO: 64) and 3′ AAV2 ITR (130 bp) (SEQ ID NO: 72).

In some embodiments, Construct C3 is 4491 bp in length (SEQ ID NO: 33) and comprises the following elements in 5′ to 3′: hTNNT2 promoter (544 bp) (SEQ ID NO: 52), wild type hMYBPC3 (SEQ ID NO: 43), and mini poly A (57 bp) (SEQ ID NO: 64). In further embodiments, Construct C3 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C4 is 4950 bp in length (SEQ ID NO: 38) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (130 bp) (SEQ ID NO: 71).

In some embodiments, Construct C4 is 4980 bp in length (SEQ ID NO: 37) and comprises the following elements in 5′ to 3′: 5′ AAV2-ITR (145 bp) (SEQ ID NO: 68), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (145 bp) (SEQ ID NO: 72).

In some embodiments, Construct C4 is 4950 bp in length (SEQ ID NO: 110) and comprises the following elements in 5′ to 3′: 5′ AAV2-ITR (130 bp) (SEQ ID NO: 69), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (130 bp) (SEQ ID NO: 73).

In some embodiments, Construct C4 is 4980 bp in length (SEQ ID NO: 111) and comprises the following elements in 5′ to 3′: 5′ AAV2-ITR (145 bp) (SEQ ID NO: 70), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (145 bp) (SEQ ID NO: 74).

In some embodiments, Construct C4 is 4950 bp in length (SEQ ID NO: 112) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (130 bp) (SEQ ID NO: 73).

In some embodiments, Construct C4 is 4980 bp in length (SEQ ID NO: 113) and comprises the following elements in 5′ to 3′: 5′ AAV2-ITR (145 bp) (SEQ ID NO: 68), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (145 bp) (SEQ ID NO: 74).

In some embodiments, Construct C4 is 4950 bp in length (SEQ ID NO: 114) and comprises the following elements in 5′ to 3′: 5′ AAV2-ITR (130 bp) (SEQ ID NO: 69), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (130 bp) (SEQ ID NO: 71).

In some embodiments, Construct C4 is 4980 bp in length (SEQ ID NO: 115) and comprises the following elements in 5′ to 3′: 5′ AAV2-ITR (145 bp) (SEQ ID NO: 70), hTNNT2 promoter (544 bp) (SEQ ID NO: 52), chimeric intron (133 bp) (SEQ ID NO: 58), wild type hMYBPC3 (SEQ ID NO: 43), mini poly A (57 bp) (SEQ ID NO: 64) and 3′ AAV2 ITR (145 bp) (SEQ ID NO: 72).

In some embodiments, Construct C4 is 4640 bp in length (SEQ ID NO: 36) and comprises the following elements in 5′ to 3′: hTNNT2 promoter (544 bp) (SEQ ID NO: 52), chimeric intron (133 bp) (SEQ ID NO: 58), wild type hMYBPC3 (SEQ ID NO: 43), and mini poly A (57 bp) (SEQ ID NO: 64). In further embodiments, Construct C4 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct C5 is 4950 bp in length (SEQ ID NO: 41) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (130 bp) (SEQ ID NO: 71).

In some embodiments, Construct C5 is 4980 bp in length (SEQ ID NO: 40) and comprises the following elements in 5′ to 3′: 5′ AAV2-ITR (145 bp) (SEQ ID NO: 68), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (145 bp) (SEQ ID NO: 72).

In some embodiments, Construct C5 is 4950 bp in length (SEQ ID NO: 116) and comprises the following elements in 5′ to 3′: 5′ AAV2-ITR (130 bp) (SEQ ID NO: 69), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (130 bp) (SEQ ID NO: 73).

In some embodiments, Construct C5 is 4980 bp in length (SEQ ID NO: 117) and comprises the following elements in 5′ to 3′: 5′ AAV2-ITR (145 bp) (SEQ ID NO: 70), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (145 bp) (SEQ ID NO: 74).

In some embodiments, Construct C5 is 4950 bp in length (SEQ ID NO: 118) and comprises the following elements in 5′ to 3′: 5′ AAV2-ITR (130 bp) (SEQ ID NO: 67), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (130 bp) (SEQ ID NO: 73).

In some embodiments, Construct C5 is 4980 bp in length (SEQ ID NO: 119) and comprises the following elements in 5′ to 3′: 5′ AAV2-ITR (145 bp) (SEQ ID NO: 68), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (145 bp) (SEQ ID NO: 74).

In some embodiments, Construct C5 is 4950 bp in length (SEQ ID NO: 120) and comprises the following elements in 5′ to 3′: 5′ AAV2-ITR (130 bp) (SEQ ID NO: 69), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (130 bp) (SEQ ID NO: 71).

In some embodiments, Construct C5 is 4980 bp in length (SEQ ID NO: 121) and comprises the following elements in 5′ to 3′: 5′ AAV2-ITR (145 bp) (SEQ ID NO: 70), hTNNT2 promoter (544 bp) (SEQ ID NO: 52), chimeric intron (133 bp) (SEQ ID NO: 58), CpG free hMYBPC3 (SEQ ID NO: 45), mini poly A (57 bp) (SEQ ID NO: 64) and 3′ AAV2 ITR (145 bp) (SEQ ID NO: 72).

In some embodiments, Construct C5 is 4806 bp in length (SEQ ID NO: 39) and comprises the following elements in 5′ to 3′: hTNNT2 promoter (544 bp) (SEQ ID NO: 52), chimeric intron (133 bp) (SEQ ID NO: 58), CpG free hMYBPC3 (SEQ ID NO: 45), and mini poly A (57 bp) (SEQ ID NO: 64). In further embodiments, Construct C5 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A1 is 5074 bp in length (SEQ ID NO: 5) and comprises the following elements in 5′ to 3′: 5′ AAV2-ITR (130 bp) (SEQ ID NO: 67), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (130 bp) (SEQ ID NO: 71).

In some embodiments, Construct A1 is 5104 bp in length (SEQ ID NO: 4) and comprise the following elements in 5′ to 3′: 5′ AAV2-ITR (145 bp) (SEQ ID NO: 68), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (145 bp) (SEQ ID NO: 72);

In some embodiments, Construct A1 is 5074 bp in length (SEQ ID NO: 122) and comprise the following elements in 5′ to 3′: 5′ AAV2-ITR (130 bp) (SEQ ID NO: 69), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (130 bp) (SEQ ID NO: 73);

In some embodiments, Construct A1 is 5104 bp in length (SEQ ID NO: 123) and comprise the following elements in 5′ to 3′: 5′ AAV2-ITR (145 bp) (SEQ ID NO: 70), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (145 bp) (SEQ ID NO: 74);

In some embodiments, Construct A1 is 5074 bp in length (SEQ ID NO: 124) and comprise the following elements in 5′ to 3′: 5′ AAV2-ITR (130 bp) (SEQ ID NO: 67), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (130 bp) (SEQ ID NO: 73);

In some embodiments, Construct A1 is 5104 bp in length (SEQ ID NO: 125) and comprise the following elements in 5′ to 3′: 5′ AAV2-ITR (145 bp) (SEQ ID NO: 68), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (145 bp) (SEQ ID NO: 74);

In some embodiments, Construct A1 is 5074 bp in length (SEQ ID NO: 126) and comprise the following elements in 5′ to 3′: 5′ AAV2-ITR (130 bp) (SEQ ID NO: 69), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (130 bp) (SEQ ID NO: 71);

In some embodiments, Construct A1 is 5104 bp in length (SEQ ID NO: 127) and comprise the following elements in 5′ to 3′: 5′ AAV2-ITR (145 bp) (SEQ ID NO: 70), hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), bGH poly A (227 bp) (SEQ ID NO: 61) and 3′ AAV2 ITR (145 bp) (SEQ ID NO: 72).

In some embodiments, Construct A1 is 4786 bp in length (SEQ ID NO: 3) and comprise the following elements in 5′ to 3′: hTNNT2 promoter (532 bp) (SEQ ID NO: 51), globin intron (131 bp) (SEQ ID NO: 53), HBB exon 3 (SEQ ID NO: 54); wild type hMYBPC3 (SEQ ID NO: 42), and bGH poly A (227 bp) (SEQ ID NO: 61). In further embodiments, Construct A1 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A2 is 4939 bp in length (SEQ ID NO: 8) and comprises the following elements in 5′ to 3′: 5′ AAV2-ITR (130 bp) (SEQ ID NO: 67), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 71).

In some embodiments, Construct A2 is 4969 bp in length (SEQ ID NO: 7) and comprises the following elements in 5′ to 3′: 5′ AAV2-ITR (145 bp) (SEQ ID NO: 68), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 72).

In some embodiments, Construct A2 is 4939 bp in length (SEQ ID NO: 128) and comprises the following elements in 5′ to 3′: 5′ AAV2-ITR (130 bp) (SEQ ID NO: 69), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 73).

In some embodiments, Construct A2 is 4969 bp in length (SEQ ID NO: 129) and comprises the following elements in 5′ to 3′: 5′ AAV2-ITR (145 bp) (SEQ ID NO: 70), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 74).

In some embodiments, Construct A2 is 4939 bp in length (SEQ ID NO: 130) and comprises the following elements in 5′ to 3′: 5′ AAV2-ITR (130 bp) (SEQ ID NO: 67), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 73).

In some embodiments, Construct A2 is 4969 bp in length (SEQ ID NO: 131) and comprises the following elements in 5′ to 3′: 5′ AAV2-ITR (145 bp) (SEQ ID NO: 68), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 74).

In some embodiments, Construct A2 is 4939 bp in length (SEQ ID NO: 132) and comprises the following elements in 5′ to 3′: 5′ AAV2-ITR (130 bp) (SEQ ID NO: 69), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 71).

In some embodiments, Construct A2 is 4969 bp in length (SEQ ID NO: 133) and comprises the following elements in 5′ to 3′: 5′ AAV2-ITR (145 bp) (SEQ ID NO: 70), hTNNT2 promoter (469 bp) (SEQ ID NO: 49), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 72).

In some embodiments, Construct A2 is 4663 bp in length (SEQ ID NO: 6) and comprises the following elements in 5′ to 3′: hTNNT2 promoter (469 bp) (SEQ ID NO: 49), wild type hMYBPC3 (SEQ ID NO: 42) with globin intron (131 bp) (SEQ ID NO: 53) between exons, and bGH poly A (227 bp) (SEQ ID NO: 61). In further embodiments, Construct A2 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A3 is 4939 bp in length (SEQ ID NO: 11) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (130 bp) (SEQ ID NO: 71).

In some embodiments, Construct A3 is 4969 bp in length (SEQ ID NO: 10) and comprises the following elements in 5′ to 3′: 5′ AAV2-ITR (145 bp) (SEQ ID NO: 68), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (145) (SEQ ID NO: 72).

In some embodiments, Construct A3 is 4939 bp in length (SEQ ID NO: 134) and comprises the following elements in 5′ to 3′: 5′ AAV2-ITR (130 bp) (SEQ ID NO: 69), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (130 bp) (SEQ ID NO: 73).

In some embodiments, Construct A3 is 4969 bp in length (SEQ ID NO: 135) and comprises the following elements in 5′ to 3′: 5′ AAV2-ITR (145 bp) (SEQ ID NO: 70), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (145) (SEQ ID NO: 74).

In some embodiments, Construct A3 is 4939 bp in length (SEQ ID NO: 136) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (130 bp) (SEQ ID NO: 73).

In some embodiments, Construct A3 is 4969 bp in length (SEQ ID NO: 137) and comprises the following elements in 5′ to 3′: 5′ AAV2-ITR (145 bp) (SEQ ID NO: 68), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (145) (SEQ ID NO: 74).

In some embodiments, Construct A3 is 4939 bp in length (SEQ ID NO: 138) and comprises the following elements in 5′ to 3′: 5′ AAV2-ITR (130 bp) (SEQ ID NO: 69), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (130 bp) (SEQ ID NO: 71).

In some embodiments, Construct A3 is 4969 bp in length (SEQ ID NO: 139) and comprises the following elements in 5′ to 3′: 5′ AAV2-ITR (145 bp) (SEQ ID NO: 70), hTNNT2 promoter (532 bp) (SEQ ID NO: 51), wild type hMYBPC3 (SEQ ID NO: 42) with globin intron (131 bp) (SEQ ID NO: 53) between exons, bGH poly A (169 bp) (SEQ ID NO: 59) and 3′ AAV2 ITR (145) (SEQ ID NO: 72).

In some embodiments, Construct A3 is 4663 bp in length (SEQ ID NO: 9) and comprises the following elements in 5′ to 3′: hTNNT2 promoter (532 bp) (SEQ ID NO: 51), wild type hMYBPC3 (SEQ ID NO: 42) with globin intron (131 bp) (SEQ ID NO: 53) between exons, and bGH poly A (169 bp) (SEQ ID NO: 59). In further embodiments, Construct A3 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A4 is 4939 bp in length (SEQ ID NO: 14) and comprises the following elements in 5′ to 3′: 5′ AAV2-ITR (130 bp) (SEQ ID NO: 67), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (130 bp) (SEQ ID NO: 71).

In some embodiments, Construct A4 is 4969 bp in length (SEQ ID NO: 13) and comprises the following elements in 5′ to 3′: 5′ AAV2-ITR (145 bp) (SEQ ID NO: 68), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (145 bp) (SEQ ID NO: 72).

In some embodiments, Construct A4 is 4939 bp in length (SEQ ID NO: 140) and comprises the following elements in 5′ to 3′: 5′ AAV2-ITR (130 bp) (SEQ ID NO: 69), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (130 bp) (SEQ ID NO: 73).

In some embodiments, Construct A4 is 4969 bp in length (SEQ ID NO: 141) and comprises the following elements in 5′ to 3′: 5′ AAV2-ITR (145 bp) (SEQ ID NO: 70), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (145 bp) (SEQ ID NO: 74).

In some embodiments, Construct A4 is 4939 bp in length (SEQ ID NO: 142) and comprises the following elements in 5′ to 3′: 5′ AAV2-ITR (130 bp) (SEQ ID NO: 67), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (130 bp) (SEQ ID NO: 73).

In some embodiments, Construct A4 is 4969 bp in length (SEQ ID NO: 143) and comprises the following elements in 5′ to 3′: 5′ AAV2-ITR (145 bp) (SEQ ID NO: 68), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (145 bp) (SEQ ID NO: 74).

In some embodiments, Construct A4 is 4939 bp in length (SEQ ID NO: 144) and comprises the following elements in 5′ to 3′: 5′ AAV2-ITR (130 bp) (SEQ ID NO: 69), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (130 bp) (SEQ ID NO: 71).

In some embodiments, Construct A4 is 4969 bp in length (SEQ ID NO: 145) and comprises the following elements in 5′ to 3′: 5′ AAV2-ITR (145 bp) (SEQ ID NO: 70), hTNNT2 promoter (499 bp) (SEQ ID NO: 50), wild type hMYBPC3 (SEQ ID NO: 42) with globin intron (131 bp) (SEQ ID NO: 53) between exons, bGH poly A (202 bp) (SEQ ID NO: 60) and 3′ AAV2 ITR (145 bp) (SEQ ID NO: 72).

In some embodiments, Construct A4 is 4663 bp in length (SEQ ID NO: 12) and comprises the following elements in 5′ to 3′: hTNNT2 promoter (499 bp) (SEQ ID NO: 50), wild type hMYBPC3 (SEQ ID NO: 42) with globin intron (131 bp) (SEQ ID NO: 53) between exons, and bGH poly A (202 bp) (SEQ ID NO: 60). In further embodiments, Construct A4 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A5 is 4871 bp in length (SEQ ID NO: 17) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (130 bp) (SEQ ID NO: 71).

In some embodiments, Construct A5 is 4901 bp in length (SEQ ID NO: 16) and comprises the following elements in 5′ to 3′: 5′ AAV2-ITR (145 bp) (SEQ ID NO: 67), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (145 bp) (SEQ ID NO: 71).

In some embodiments, Construct A5 is 4871 bp in length (SEQ ID NO: 146) and comprises the following elements in 5′ to 3′: 5′ AAV2-ITR (130 bp) (SEQ ID NO: 69), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (130 bp) (SEQ ID NO: 73).

In some embodiments, Construct A5 is 4901 bp in length (SEQ ID NO: 147) and comprises the following elements in 5′ to 3′: 5′ AAV2-ITR (145 bp) (SEQ ID NO: 70), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (145 bp) (SEQ ID NO: 74).

In some embodiments, Construct A5 is 4871 bp in length (SEQ ID NO: 148) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (130 bp) (SEQ ID NO: 73).

In some embodiments, Construct A5 is 4901 bp in length (SEQ ID NO: 149) and comprises the following elements in 5′ to 3′: 5′ AAV2-ITR (145 bp) (SEQ ID NO: 68), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (145 bp) (SEQ ID NO: 74).

In some embodiments, Construct A5 is 4871 bp in length (SEQ ID NO: 150) and comprises the following elements in 5′ to 3′: 5′ AAV2-ITR (130 bp) (SEQ ID NO: 69), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (130 bp) (SEQ ID NO: 71).

In some embodiments, Construct A5 is 4901 bp in length (SEQ ID NO: 151) and comprises the following elements in 5′ to 3′: 5′ AAV2-ITR (145 bp) (SEQ ID NO: 70), hTNNT2 promoter (532 bp) (SEQ ID NO: 51), wild type hMYBPC3 (SEQ ID NO: 42), bGH poly A (227 bp) (SEQ ID NO: 61) and 3′ AAV2 ITR (145 bp) (SEQ ID NO: 72).

In some embodiments, Construct A5 is 4595 bp in length (SEQ ID NO: 15) and comprises the following elements in 5′ to 3′: hTNNT2 promoter (532 bp) (SEQ ID NO: 51), wild type hMYBPC3 (SEQ ID NO: 42), and bGH poly A (227 bp) (SEQ ID NO: 61). In further embodiments, Construct A5 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A6 is 5002 bp in length (SEQ ID NO: 20) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 71).

In some embodiments, Construct A6 is 5032 bp in length (SEQ ID NO: 19) and comprises the following elements in 5′ to 3′: 5′ AAV2-ITR (145 bp) (SEQ ID NO: 68), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 72).

In some embodiments, Construct A6 is 5002 bp in length (SEQ ID NO: 152) and comprises the following elements in 5′ to 3′: 5′ AAV2-ITR (130 bp) (SEQ ID NO: 69), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 73).

In some embodiments, Construct A6 is 5032 bp in length (SEQ ID NO: 153) and comprises the following elements in 5′ to 3′: 5′ AAV2-ITR (145 bp) (SEQ ID NO: 70), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 74).

In some embodiments, Construct A6 is 5002 bp in length (SEQ ID NO: 154) and comprises the following elements in 5′ to 3′: 5′ AAV2-ITR (130 bp) (SEQ ID NO: 67), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 73).

In some embodiments, Construct A6 is 5032 bp in length (SEQ ID NO: 155) and comprises the following elements in 5′ to 3′: 5′ AAV2-ITR (145 bp) (SEQ ID NO: 68), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 74).

In some embodiments, Construct A6 is 5002 bp in length (SEQ ID NO: 156) and comprises the following elements in 5′ to 3′: 5′ AAV2-ITR (130 bp) (SEQ ID NO: 69), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (130 bp) (SEQ ID NO: 71).

In some embodiments, Construct A6 is 5032 bp in length (SEQ ID NO: 157) and comprises the following elements in 5′ to 3′: 5′ AAV2-ITR (145 bp) (SEQ ID NO: 70), hTNNT2 promoter (532 bp) (SEQ ID NO: 51), wild type hMYBPC3 (SEQ ID NO: 42) with globin intron (131 bp) (SEQ ID NO: 53) between exons, bGH poly A (227 bp) (SEQ ID NO: 61) and 3′ AAV2 ITR (145 bp) (SEQ ID NO: 72).

In some embodiments, Construct A6 is 4726 bp in length (SEQ ID NO: 18) and comprises the following elements in 5′ to 3′: hTNNT2 promoter (532 bp) (SEQ ID NO: 51), wild type hMYBPC3 (SEQ ID NO: 42) with globin intron (131 bp) (SEQ ID NO: 53) between exons, and bGH poly A (227 bp) (SEQ ID NO: 61). In further embodiments, Construct A6 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A7 is 4781 bp in length (SEQ ID NO: 23) and comprises the following elements in 5′ to 3′: 5′ AAV2-ITR (130 bp) (SEQ ID NO: 67), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 71).

In some embodiments, Construct A7 is 4811 bp in length (SEQ ID NO: 22) and comprises the following elements in 5′ to 3′: 5′ AAV2-ITR (145 bp) (SEQ ID NO: 67), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 71).

In some embodiments, Construct A7 is 4781 bp in length (SEQ ID NO: 158) and comprises the following elements in 5′ to 3′: 5′ AAV2-ITR (130 bp) (SEQ ID NO: 69), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 73).

In some embodiments, Construct A7 is 4811 bp in length (SEQ ID NO: 159) and comprises the following elements in 5′ to 3′: 5′ AAV2-ITR (145 bp) (SEQ ID NO: 70), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 74).

In some embodiments, Construct A7 is 4781 bp in length (SEQ ID NO: 160) and comprises the following elements in 5′ to 3′: 5′ AAV2-ITR (130 bp) (SEQ ID NO: 67), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 73).

In some embodiments, Construct A7 is 4811 bp in length (SEQ ID NO: 161) and comprises the following elements in 5′ to 3′: 5′ AAV2-ITR (145 bp) (SEQ ID NO: 68), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 74).

In some embodiments, Construct A7 is 4781 bp in length (SEQ ID NO: 162) and comprises the following elements in 5′ to 3′: 5′ AAV2-ITR (130 bp) (SEQ ID NO: 69), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 71).

In some embodiments, Construct A7 is 4811 bp in length (SEQ ID NO: 163) and comprises the following elements in 5′ to 3′: 5′ AAV2-ITR (145 bp) (SEQ ID NO: 70), hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 72).

In some embodiments, Construct A7 is 4505 bp in length (SEQ ID NO: 21) and comprises the following elements in 5′ to 3′: hTNNT2 promoter (469 bp) (SEQ ID NO: 49), Kozac sequence, wild type hMYBPC3 (SEQ ID NO: 42) with globin intron (131 bp) (SEQ ID NO: 53) between exons, mini poly A (57 bp) (SEQ ID NO: 64), and 3′-UTR sequence. In further embodiments, Construct A7 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

Construct A8 is 4844 bp in length (SEQ ID NO: 26) and comprises the following elements in 5′ to 3′: 5′ AAV2-ITR (130 bp) (SEQ ID NO: 67), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 71).

In some embodiments, Construct A8 is 4874 bp in length (SEQ ID NO: 25) and comprises the following elements in 5′ to 3′: 5′ AAV2-ITR (145 bp) (SEQ ID NO: 67), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 71).

In some embodiments, Construct A8 is 4844 bp in length (SEQ ID NO: 164) and comprises the following elements in 5′ to 3′: 5′ AAV2-ITR (130 bp) (SEQ ID NO: 69), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 73).

In some embodiments, Construct A8 is 4874 bp in length (SEQ ID NO: 165) and comprises the following elements in 5′ to 3′: 5′ AAV2-ITR (145 bp) (SEQ ID NO: 70), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 72).

In some embodiments, Construct A8 is 4844 bp in length (SEQ ID NO: 166) and comprises the following elements in 5′ to 3′: 5′ AAV2-ITR (130 bp) (SEQ ID NO: 67), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 73).

In some embodiments, Construct A8 is 4874 bp in length (SEQ ID NO: 167) and comprises the following elements in 5′ to 3′: 5′ AAV2-ITR (145 bp) (SEQ ID NO: 68), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 73).

In some embodiments, Construct A8 is 4844 bp in length (SEQ ID NO: 168) and comprises the following elements in 5′ to 3′: 5′ AAV2-ITR (130 bp) (SEQ ID NO: 69), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (130 bp) (SEQ ID NO: 71).

In some embodiments, Construct A8 is 4874 bp in length (SEQ ID NO: 169) and comprises the following elements in 5′ to 3′: 5′ AAV2-ITR (145 bp) (SEQ ID NO: 70), hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), 3′-UTR sequence and 3′ AAV2-ITR (145 bp) (SEQ ID NO: 72).

In some embodiments, Construct A8 is 4568 bp in length (SEQ ID NO: 24) and comprises the following elements in 5′ to 3′: hTNNT2 promoter (532 bp) (SEQ ID NO: 51), Kozac sequence, wild type hMYBPC3 (SEQ ID NO: 42) with globin intron (131 bp) (SEQ ID NO: 53) between exons, mini poly A (57 bp) (SEQ ID NO: 64), and 3′-UTR sequence. In further embodiments, Construct A8 optionally comprises any of the 5′ AAV2-ITR sequences of SEQ ID NO: 67-70 (or the complement thereof) and/or any of the 3′ AAV2-ITR sequences of SEQ ID NO: 71-74 (or the complement thereof), or fragments thereof.

In any of the foregoing embodiments, the vector construct comprises at least one ITR sequence. Example ITR sequences include but are not limited to SEQ ID Nos: 67-74 including any complementary sequences and/or combinations thereof.

Polynucleotides and polypeptides including modified forms can be made using various standard cloning, recombinant DNA technology, via cell expression or in vitro translation and chemical synthesis techniques known to those of skill in the art (Sambrook et al., Molecular Cloning: A Laboratory Manual, 2^{nd} edition).

### Methods of Gene Delivery.

Also provided is a method of using vector construct or AAV particle as described herein to deliver a gene encoding the protein of interest. In one embodiment, a gene delivery vector may be a viral gene delivery vector, such as a viral particle, or a non-viral gene delivery vector, such as a vector construct or nucleic acid encoding the protein of interest. Viral vectors include lenti-, adeno-, herpes viral vectors. It is preferably a recombinant adeno-associated viral (rAAV) vector. Alternatively, non-viral systems may be used, including using naked DNA (with or without chromatin attachment regions) or conjugated DNA that is introduced into cells by various transfection methods such as lipids or electroporation.

Non-limiting examples of a vector construct as described herein include any of SEQ ID Nos: 3-41 or 92-169.

In some embodiments, the vector construct or AAV vector genome comprises a nucleotide sequence having at least about 80%, 85% 90%, at least about 95%, at least about 96%, at least about 97%, at least about 98%, at least about 99%, or more, sequence identity to any of SEQ ID Nos: 3-41 or 92-169 (over the full length of SEQ ID Nos: 3-41 or 92-169, respectively). In some embodiments, the vector construct comprises a nucleotide sequence having at least about 85% sequence identity to any of SEQ ID Nos: 3-41 or 92-169. Preferably, the vector construct or AAV vector genome of the AAV particle comprises a nucleotide sequence having at least about 95.5%, 96%, 96.5%, 97%, 97.5%, 98%, 98.5%, 99%, 99.5% or more, sequence identity to any of SEQ ID Nos: 3-41 or 92-169. Even more preferably, the nucleotide sequence of the vector construct is at least 97% or 98% or 99% or more identical to any of SEQ ID Nos: 3-41 or 92-169. In other embodiments, the vector construct comprises a nucleotide sequence at least 97%, 98% or 99% identical to a nucleotide sequence that is complementary to or is a negative (−) strand of any of SEQ ID NO: 3-41 or 92-169.

In some embodiments, the vector construct comprises a nucleotide sequence at least 97%, 98% or 99% identical to any of SEQ ID NO: 3, 6, 9, 12, 15, 18, 21, 24, 27, 30, 33, 36, or 39. In other embodiments, the vector construct comprises a nucleotide sequence at least 97%, 98% or 99% identical to a nucleotide sequence that is complementary to or is a negative (−) strand of any of SEQ ID NO: 3, 6, 9, 12, 15, 18, 21, 24, 27, 30, 33, 36, or 39.

In some embodiments, the vector construct comprises a nucleotide sequence at least 97%, 98% or 99% identical to any of SEQ ID NO: 4, 7, 10, 13, 16, 19, 22, 25, 28, 31, 34, 37, or 40. In other embodiments, the vector construct comprises a nucleotide sequence at least 97%, 98% or 99% identical to a nucleotide sequence that is complementary to or is a negative (−) strand of any of SEQ ID NO: 4, 7, 10, 13, 16, 19, 22, 25, 28, 31, 34, 37, or 40.

In some embodiments, the vector construct comprises a nucleotide sequence at least 97%, 98% or 99% identical to any of SEQ ID NO: 5, 8, 11, 14, 17, 20, 23, 26, 29, 32, 35, 38, or 41. In other embodiments, the vector construct comprises a nucleotide sequence at least 97%, 98% or 99% identical to a nucleotide sequence that is complementary to or is a negative (−) strand ofany of 5, 8, 11, 14, 17, 20, 23, 26, 29, 32, 35, 38, or 41.

The present disclosure finds use in both veterinary and medical applications. Suitable subjects for gene delivery methods as described herein include both avians and mammals, with mammals being preferred and humans being most preferred. Human subjects include neonates, infants, juveniles, and adults.

### Non-Viral Gene Delivery.

Non-viral gene delivery may be carried out using naked DNA which is the simplest method of non-viral transfection. It may be possible, for example, to administer the vector constructs provided herein using naked plasmid DNA. Alternatively, the vector constructs may be delivered using methods involving electroporation, sonoporation or the use of a “gene gun”, which shoots DNA coated gold particles into the cell using, for example, high pressure gas or an inverted .22 calibre gun (Helios® Gene Gun System (BIO-RAD)), microinjection, lasers, elevated temperature, ultrasound, hydrodynamic gene transfer, magnetotransfection, chemical transfection (e.g., calcium phosphate, DEAE-dextran), liposomes, lipoplexes, dendrimers, lipid nanoparticles or inorganic nanoparticles, all of which are known in the art.

To improve the delivery of a vector construct into a cell, it may be necessary to protect it from damage and to facilitate its entry into the cell. To this end, lipoplexes and polyplexes may be used that have the ability to protect a nucleic acid from undesirable degradation during the transfection process.

Vector constructs may be coated with lipids in an organized structure such as a micelle or a liposome. When the organized structure is complexed with DNA it is called a lipoplex. Anionic and neutral lipids may be used for the construction of lipoplexes for synthetic vectors. In one embodiment, cationic lipids, due to their positive charge, may be used to condense negatively charged DNA molecules so as to facilitate the encapsulation of DNA into liposomes. If may be necessary to add helper lipids (usually electroneutral lipids, such as DOPE) to cationic lipids so as to form lipoplexes (Dabkowska et al., J. R. Soc. Interface. 9(68): 548-61 (2012).

In certain embodiments, complexes of polymers with DNA, called polyplexes, may be used to deliver a vector construct. Most polyplexes consist of cationic polymers and their production is regulated by ionic interactions. Polyplexes typically cannot release their DNA load into the cytoplasm. Thus, co-transfection with endosome-lytic agents (to lyse the endosome that is made during endocytosis, the process by which the polyplex enters the cell), such as inactivated adenovirus, may be necessary (Akinc et al., J. Gene Medic. 7 (5): 657-63).

In certain embodiments, hybrid methods may be used to deliver a vector construct that combines two or more techniques. Virosomes are one example; they combine liposomes with an inactivated HIV or influenza virus. In another embodiment, other methods involve mixing other viral vectors with cationic lipids or hybridizing viruses and may be used to deliver a nucleic acid (Khan, Firdos Alam, Biotechnology Fundamentals, CRC Press, Nov. 18, 2015, p. 395).

In certain embodiments, a dendrimer may be used to deliver a vector construct, in particular, a cationic dendrimer, i.e., one with a positive surface charge. When in the presence of genetic material as DNA or RNA, charge complementarity leads to a temporary association of the nucleic acid with the cationic dendrimer. On reaching its destination the dendrimer-nucleic acid complex is then imported into the cell via endocytosis (Amiji, Mansoor M. ed., Polymeric Gene Delivery: Principles and Applications, CRC Press, Sep. 29, 2004, p. 142.)

### Viral Particles.

In one embodiment, a suitable viral gene delivery vector such as a viral particle may be used to deliver a nucleic acid. In certain embodiments, viral gene delivery vectors suitable for use herein may be a parvovirus, an adenovirus, a retrovirus, a gamma-retrovirus, a lentivirus, a herpes simplex virus, a vaccinia virus, a measles virus, a vesicular stomatitis virus, a polio virus or a reovirus. The parvovirus may be an adenovirus-associated virus (AAV).

Accordingly, the present disclosure provides viral particles for use as gene delivery vectors (comprising a vector construct provided herein) based on animal parvoviruses, in particular dependoviruses such as infectious human or simian AAV, and the components thereof (e.g., an animal parvovirus genome) for introduction and/or expression of a cMyBP-C protein in a mammalian cell. The term “parvoviral” as used herein thus encompasses dependoviruses such as any type of AAV.

Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, and 6), primates (e.g., serotypes 1 and 4), and related viruses that infect other warm-blooded animals (e.g., bovine, canine, equine, mice, rats, and ovine adeno-associated viruses) in addition to birds and reptiles. Further information on parvoviruses and other members of the Parvoviridae is described in Kenneth I. Berns, “Parvoviridae: The Viruses and Their Replication,” Chapter 69 in Fields Virology (3d Ed. 1996). For convenience the present disclosure is further exemplified and described herein by reference to AAV. It is, however, understood that the present disclosure is not limited to AAV but may equally be applied to other parvoviruses.

Production of AAV particles requires AAV “rep” and “cap” genes, which are genes encoding replication and encapsidation proteins, respectively. AAV rep and cap genes have been found in all AAV serotypes examined to date, and are described herein and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap genes for use herein encode Cap proteins which are capable of packaging AAV vectors in the presence of rep and adeno helper function and are capable of binding target cellular receptors. In some embodiments, the AAV cap gene encodes a capsid protein having an amino acid sequence derived from a particular AAV serotype.

The AAV sequences employed for the production of AAV can be derived from the genome of any AAV serotype. Generally, the AAV serotypes have genomic sequences of significant homology at the amino acid and the nucleic acid levels, provide a similar set of genetic functions, produce virions which are essentially physically and functionally equivalent, and replicate and assemble by practically identical mechanisms. For the genomic sequence of AAV serotypes and a discussion of the genomic similarities. (See, e.g., GenBank Accession number U89790; GenBank Accession number J01901; GenBank Accession number AF043303; GenBank Accession number AF085716; Chiorini et al., J. Virol. 71: 6823-33 (1997); Srivastava et al., J. Virol. 45: 555-64 (1983); Chiorini et al., J. Virol. 73: 1309-19 (1999); Rutledge et al., J. Virol. 72: 309-19 (1998); and Wu et al., J. Virol. 74: 8635-47(2000)).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The assembly-activating protein (AAP) rapidly chaperones capsid assembly and prevents degradation of free capsid proteins (Grosse et al., J. Virol. 91(20): e01198-17 (2017). The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter. The ITRs employed in the vectors of the present embodiment may correspond to the same serotype as the associated cap genes, or may differ. In one embodiment, the ITRs employed herein correspond to an AAV2 serotype and the cap genes correspond to an AAV5 serotype.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1, 5 or 8) and the Rep and/or ITR sequences of another AAV serotype (e.g., AAV2). Such pseudotyped rAAV particles are a part of the present disclosure.

The AAV particles described herein (and the encoding AAV vector genomes) may comprise any of the capsid proteins described in WO-2018/022608 or WO-2019/222136, incorporated by reference herein in its entirety for its disclosure of human and simian AAV capsids and their properties such as transduction efficiency, tissue tropism, glycan-binding, and resistance to neutralization by IVIG, including but not limited to any of the capsids in the sequence listing and variants thereof, e.g., with chimeric swapped variable regions and/or glycan binding sequences and/or GH loop.

In one embodiment, the AAV ITR sequences for use in the context of the present disclosure are derived from AAV1, AAV2, AAV4 and/or AAV6. Likewise, the Rep (e.g., Rep78 and Rep52) coding sequences are in one embodiment derived from AAV1, AAV2, AAV4 and/or AAV6. The sequences coding for the VP1, VP2, and VP3 capsid proteins for use in the context of the present disclosure may however be taken from any serotype, such as from AAV1, AAV2, AAV3, AAV4, AAV5, AAV6, AAV7, AAV8, AAV9, AAV10, AAV11 or AAV12, or from simian AAVs, including any of the capsid proteins described in WO 2018/022608 or PCT/US19/32097, or newly developed AAV-like particles obtained by e.g., capsid shuffling techniques and AAV capsid libraries, or any capsid at least 90% identical to any of SEQ ID Nos: 75-91.

For example, the amino acid sequences of various capsids are published. See, e.g.,
AAVRh.1/hu.14/AAV9 AAS99264.1 (SEQ ID NO: 75)
AAVRh.8 SEQ97 of U.S. Pat. Pub. 2013/0045186 (SEQ ID NO: 76)
AAVRh.10 SEQ81 of U.S. Pat. Pub. 2013/0045186 (SEQ ID NO: 77)
AAVRh.74 SEQ 1 of Int'l. Pat. Pub. WO 2013/123503 (SEQ ID NO: 78)
AAV1 AAB_95452.1 (SEQ ID NO: 79)
AAV2 YP_680426.1 (SEQ ID NO: 80)
AAV3 NP_043941.1 (SEQ ID NO: 81)
AAV3B AAB95452.1 (SEQ ID NO: 82)
AAV4 NP_044927.1 (SEQ ID NO: 83)
AAV5 YP_068409.1 (SEQ ID NO: 84)
AAV6 AAB95450.1 (SEQ ID NO: 85)
AAV7 YP_077178.1 (SEQ ID NO: 86)
AAV8 YP_077180.1 (SEQ ID NO: 87)
AAV10 AAT46337.1 (SEQ ID NO: 88)
AAV11 AAT46339.1 (SEQ ID NO: 89)
AAV12 AB116639.1 (SEQ ID NO: 90)
AAV13 ABZ10812.1 (SEQ ID NO: 91)

Modified “AAV” sequences also can be used in the context of the present disclosure, e.g., for the production of AAV gene therapy vectors. Such modified sequences e.g., sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP, can be used in place of wild-type AAV ITR, Rep, or VP sequences.

In some embodiments, a nucleic acid sequence encoding an AAV capsid protein is operably linked to expression control sequences for expression in a specific cell type, such as Sf9 or HEK cells. Techniques known to one skilled in the art for expressing foreign genes in insect host cells or mammalian host cells can be used to practice the embodiment. Methodology for molecular engineering and expression of polypeptides in insect cells is described, for example, in Summers and Smith (1986) A Manual of Methods for Baculovirus Vectors and Insect Culture Procedures, Texas Agricultural Experimental Station Bull. No. 7555, College Station, Tex.; Luckow (1991) In Prokop et al., Cloning and Expression of Heterologous Genes in Insect Cells with Baculovirus Vectors' Recombinant DNA Technology and Applications, 97-152; King, L. A. and R. D. Possee (1992) The baculovirus expression system, Chapman and Hall, United Kingdom; O'Reilly, D. R., L. K. Miller, V. A. Luckow (1992) Baculovirus Expression Vectors: A Laboratory Manual, New York; W.H. Freeman and Richardson, C. D. (1995) Baculovirus Expression Protocols, Methods in Molecular Biology, volume 39; U.S. Pat. No. 4,745,051; US-2003148506; and WO-03/074714, all of which are incorporated by reference in their entireties. A particularly suitable promoter for transcription of a nucleotide sequence encoding an AAV capsid protein is e.g., the polyhedron promoter. However, other promoters that are active in insect cells are known in the art, e.g., the p10, p35 or IE-1 promoters and further promoters described in the above references are also contemplated.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, NJ (1995); O'Reilly et al., BACULOVIRUS EXPRESSION VECTORS, A LABORATORY MANUAL, Oxford Univ. Press (1994); Samulski et al., J. Virol. 63: 3822-8 (1989); Kajigaya et al., Proc. Nat'l. Acad. Sci. USA, 88: 4646-50 (1991); Ruffing et al., J. Virol. 66: 6922-30 (1992); Kirnbauer et al., Virol. 219: 37-44 (1996); Zhao et al., Virol. 272: 382-93 (2000); and U.S. Pat. No. 6,204,059). In some embodiments, the nucleic acid construct encoding AAV proteins (e.g., AAV rep or cap proteins) in insect cells is an insect cell-compatible vector. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In one embodiment, the vector is a baculovirus, i.e., the construct is a baculoviral vector. Baculoviral vectors and methods for their use are described in the above cited references on molecular engineering of insect cells.

### Methods of Producing Recombinant AAV Particles

The present disclosure provides materials and methods for producing recombinant AAV particles in insect or mammalian cells that comprise any of the vector constructs described herein. In some embodiments, the vector construct further comprises a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the vector construct further comprises a posttranscriptional regulatory element downstream of the restriction site and upstream of the 3′ AAV ITR. In some embodiments, the vector construct further comprises a polynucleotide inserted at the restriction site and operably linked with the promoter, where the polynucleotide comprises the coding region of a protein of interest. As a skilled artisan will appreciate, any one of the AAV vector constructs disclosed in the present application can be used in methods to produce the recombinant AAV particle.

In some embodiments, the helper functions for producing AAV are provided by one or more helper plasmids or helper viruses comprising adenoviral or baculoviral helper genes. Non-limiting examples of the adenoviral or baculoviral helper genes include, but are not limited to, E1A, E1B, E2A, E4 and VA, which can provide helper functions to AAV packaging.

Helper viruses of AAV are known in the art and include, for example, viruses from the family Adenoviridae and the family Herpes viridae. Examples of helper viruses of AAV include, but are not limited to, SadV-13 helper virus and SadV-13-like helper virus described in US Publication No. 20110201088 (the disclosure of which is incorporated herein by reference), and helper vectors pHELP (Applied Viromics). A skilled artisan will appreciate that any helper virus or helper plasmid of AAV that can provide adequate helper function to AAV can be used herein.

In some embodiments, the AAV cap genes are present in a plasmid. The plasmid can further comprise an AAV rep gene which may or may not correspond to the same serotype as the cap genes. The cap genes and/or rep gene from any AAV serotype described herein (including, but not limited to, AAV1, AAV2, AAV4, AAV5, AAV6, AAV7, AAV8, AAV9, AAV10, AAV 11, AAV12, AAV13 and any variants thereof) can be used to produce the recombinant AAV. In some embodiments, the AAV cap genes encode a capsid from serotype 1, serotype 2, serotype 4, serotype 5, serotype 6, serotype 7, serotype 8, serotype 9, serotype 10, serotype 11, serotype 12, serotype 13 or a variant thereof.

In some embodiments, the insect or mammalian cell can be transfected with the helper plasmid or helper virus, the vector construct and the plasmid encoding the AAV cap genes; and the recombinant AAV virus can be collected at various time points after co-transfection. For example, the recombinant AAV virus can be collected at about 12 hours, about 24 hours, about 36 hours, about 48 hours, about 72 hours, about 96 hours, about 120 hours, or a time between any of these two time points after the co-transfection.

Recombinant AAV particles can also be produced using any conventional methods known in the art suitable for producing infectious recombinant AAV. In some instances, a recombinant AAV can be produced by using an insect or mammalian cell that stably expresses some of the necessary components for AAV particle production. For example, a plasmid (or multiple plasmids) comprising AAV rep and cap genes, and a selectable marker, such as a neomycin resistance gene, can be integrated into the genome of the cell. The insect or mammalian cell can then be co-infected with a helper virus (e.g., adenovirus or baculovirus providing the helper functions) and the viral vector construct comprising the 5′ and 3′ AAV ITR (and the nucleotide sequence encoding the heterologous protein, if desired). The advantages of this method are that the cells are selectable and are suitable for large-scale production of the recombinant AAV particle. As another non-limiting example, adenovirus or baculovirus rather than plasmids can be used to introduce rep and cap genes into packaging cells. As yet another non-limiting example, both the viral vector construct containing the 5′ and 3′ AAV ITRs and the rep-cap genes can be stably integrated into the DNA of producer cells, and the helper function can be provided by a wild-type adenovirus to produce the recombinant AAV.

In one aspect, provided herein are methods for the production of an AAV particle, useful as a gene delivery vector, the method comprising the steps of:
(a) providing a cell permissive for AAV replication (e.g., an insect cell or a mammalian cell) with one or more nucleic acid constructs comprising:
   (i) a nucleic acid molecule (e.g., recombinant vector construct) provided herein that is flanked by at least one AAV Inverted terminal repeat nucleotide sequence;
   (ii) a nucleotide sequence encoding one or more AAV Rep proteins which is operably linked to a promoter that is capable of driving expression of the Rep protein(s) in the cell;
   (iii) a nucleotide sequence encoding one or more AAV capsid proteins which is operably linked to a promoter that is capable of driving expression of the capsid protein(s) in the cell;
   (iv) and optionally AAP and MAAP contained in the VP2/3 mRNA
(b) culturing the cell defined in (a) under conditions conducive to the expression of the Rep and the capsid proteins; and,
optionally, (c) recovering the AAV gene delivery vector, and
optionally (d) purifying the AAV particle. For example, the recombinant vector construct of (i) comprises (1) at least one AAV ITR, (2) a heterologous cardiomyocyte-specific transcription regulatory region as described herein, and (3) a nucleic acid encoding a functional cMyBP-C. Preferably the recombinant vector construct of (i) comprises both a 5′ and 3′ AAV ITR.

Typically then, a method provided herein for producing a AAV gene delivery vector comprises: providing to a cell permissive for AAV replication (a) a nucleotide sequence encoding a template for producing vector genome, e.g., vector construct of the present disclosure (as described in detail herein); (b) nucleotide sequences sufficient for replication of the template to produce a vector genome (the first expression cassette defined above); (c) nucleotide sequences sufficient to package the vector genome into an AAV capsid (the second expression cassette defined above), under conditions sufficient for replication and packaging of the vector genome into the AAV capsid, whereby AAV particles comprising the vector genome encapsulated within the AAV capsid are produced in the cell.

Transient transfection of adherent HEK293 cells (Chahal et al., J. Virol. Meth. 196: 163-73 (2014)) and transfection of Sf9 cells, using the baculovirus expression vector system (BEVS) (Mietzsch et al., Hum. Gene Ther. 25: 212-22 (2014)), are two of the most commonly used methods to produce AAV vectors.

The viral particles comprising the vector constructs described herein may be produced using any cell type such as mammalian and invertebrate cell types which allows for production of AAV or biologic products and which can be maintained in culture.

There are a number of methods for generating AAV viral particles: for example, but not limited to, transfection using vector and AAV helper sequences in conjunction with coinfection with one of the AAV helper viruses (e.g., adenovirus, herpesvirus, or vaccinia virus) or transfection with a recombinant AAV vector, an AAV helper vector, and an accessory function vector. Methods of making AAV viral particles are described in e.g., U.S. Pat. Nos. U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, WO2001023597, WO2015191508, WO2019217513, WO2018022608, WO2019222136, WO2020232044, WO2019222132; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. For detailed descriptions of methods for generating AAV viral particles see, for example, U.S. Pat. Nos. 6,001,650, 6,004,797, and 9,504,762, each herein incorporated by reference in its entirety. In one embodiment, a triple transfection method (see, e.g., U.S. Pat. No. 6,001,650, herein incorporated by reference in its entirety) is used to produce AAV viral particles. This method does not require the use of an infectious helper virus, enabling AAV viral particles to be produced without any detectable helper virus present. This is accomplished by use of three vectors for AAV viral particle production, namely an AAV helper function vector, an accessory function vector, and an AAV viral particle expression vector. One of skill in the art will appreciate, however, that the nucleic acid sequences encoded by these vectors can be provided on two or more vectors in various combinations. In other embodiments, the host cell can be transfected with the helper plasmid or helper virus, the viral construct and the plasmid encoding the AAV cap genes; and the AAV viral particles can be collected at various time points after co-transfection.

For example, wild-type AAV and helper viruses may be used to provide the necessary replicative functions for producing AAV viral particles (see, e.g., U.S. Pat. No. 5,139,941, herein incorporated by reference in its entirety). Alternatively, a plasmid, containing helper function genes, in combination with infection by one of the well-known helper viruses can be used as the source of replicative functions (see e.g., U.S. Pat. Nos. 5,622,856 and 5,139,941, both herein incorporated by reference in their entireties). Similarly, a plasmid, containing accessory function genes can be used in combination with infection by wild-type AAV, to provide the necessary replicative functions. Other approaches, described herein and/or well known in the art, can also be employed by the skilled artisan to produce AAV viral particles.

The term “vector” is understood to refer to any genetic element, such as a plasmid, phage, transposon, cosmid, bacmid, mini-plasmid (e.g., plasmid devoid of bacterial elements), Doggybone DNA (e.g., minimal, closed-linear constructs), chromosome, virus, virion (e.g., baculovirus), etc., which is capable of replication when associated with the proper control elements and which can transfer gene sequences between cells. A “mammalian cell-compatible vector” or “vector” as used herein refers to a nucleic acid molecule capable of productive transformation or transfection of a mammal or mammalian cell. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. Vectors and methods for their use are described in the above cited references on molecular engineering of cells.

The vector from which the cell generates an rAAV vector genome may contain a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. The vector may also contain a posttranscriptional regulatory element downstream of the restriction site and upstream of the 3′ AAV ITR. The viral construct may further comprise a polynucleotide inserted at the restriction site and operably linked with the promoter, where the polynucleotide comprises the coding region of a protein of interest. In some embodiments, the viral construct further includes a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the viral construct further incudes a posttranscriptional regulatory element downstream of the restriction site and upstream of the 3′ AAV ITR. In some embodiments, the viral construct further includes a polynucleotide inserted at the restriction site and operably linked with the promoter, where the polynucleotide includes the coding region of a protein of interest. As a skilled artisan will appreciate, any one of the AAV vectors disclosed in the present application can be used in the method as the viral construct to produce the rAAV virions.

The term “AAV helper” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The capsid (Cap) expression products supply necessary packaging functions. AAV helper functions are used herein to complement AAV functions in trans that are missing from AAV vector genomes.

For production, cells with AAV helper functions produce recombinant capsid proteins sufficient to form a capsid. This includes at least VP1 and VP3 proteins, but more typically, all three of VP1, VP2, and VP3 proteins, as found in native AAV. The sequence of the capsid proteins determines the serotype of the AAV virions produced by the host cell. Capsids useful in the invention include those derived from a number of AAV serotypes, including 1, 2, 3, 3B, 4, 5, 6, 7, 8, 9, 10, 11, 12, 13 or mixed serotypes (see, e.g., U.S. Pat. No. 8,318,480 for its disclosure of non-natural mixed serotypes). The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. Because of wide construct availability and extensive characterization, illustrative AAV vectors disclosed below are derived from serotype 2. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In various embodiments, nucleotide sequences encoding VP proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters such as the SV40 promoter, CMV promoter, RSV promoter, UBC promoter, EF1A promoter, PGK promoter, dihydrofolate reductase promoter, the b-actin promoter, TRE (Tet, Tet-On, Tet-Off) promoter, Cumate controlled systems (CuR/CuO) (See US2004/0205834), the temperature-induced HSP70 promoter, p5 promoter, p10 promoter, p19 promoter, and the p40 promoter. In another example, the nucleotide sequences can be operably linked to baculoviral promoters such as the polyhedrin (Polh) promoter, ΔIE1 promoter, p5 promoter, p10 promoter, p19 promoter, the p40 promoter, metallothionein promoter, 39K promoter, p6.9 promoter, and orf46 promoter.

For production, cells with AAV helper functions produce Rep proteins to promote production of rAAV. It has been found that infectious particles can be produced when at least one large Rep protein (Rep78 or Rep68) and at least one small Rep protein (Rep52 and Rep40) are expressed in cells. In a specific embodiment all four of Rep 78, Rep68, Rep52 and Rep 40 are expressed. Alternately, Rep78 and Rep52, Rep78 and Rep40, Rep 68 and Rep52, or Rep68 and Rep40 are expressed. Examples below demonstrate the use of the Rep78/Rep52 combination. Rep proteins can be derived from AAV-2 or other serotypes. In various embodiments, nucleotide sequences encoding Rep proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters such as the SV40 promoter, CMV promoter, RSV promoter, UBC promoter, EF1A promoter, PGK promoter, dihydrofolate reductase promoter, the b-actin promoter, TRE (Tet, Tet-On, Tet-Off) promoter, Cumate controlled systems (CuR/CuO) (See US2004/0205834), and the temperature-induced HSP70 promoter, p5 promoter, p10 promoter, p19 promoter, and the p40 promoter. In other examples, the nucleotide sequences can be operably linked to baculoviral promoters such as the polyhedrin (Polh) promoter, ΔIE1 promoter, p5 promoter, p10 promoter, p19 promoter, the p40 promoter, metallothionein promoter, 39K promoter, p6.9 promoter, and orf46 promoter.

In some embodiments, the AAV cap genes are present in a plasmid or bacmid. The plasmid can further include an AAV rep gene which may or may not correspond to the same serotype as the cap genes. The cap genes and/or rep gene from any AAV serotype.

Cells with AAV helper functions can also produce assembly-activating proteins (AAP), which help assemble capsids. In various embodiments, nucleotide sequences encoding AAP can be operably linked to a suitable expression control sequence. For example, the nucleotide sequences can be operably linked to eukaryotic promoters. In other examples, the nucleotide sequences can be operably linked to baculoviral promoters such as the polyhedrin (Polh) promoter, ΔIE1 promoter, p5 promoter, p10 promoter p19 promoter, the p40 promoter, metallothionein promoter, 39K promoter, p6.9 promoter, and orf46 promoter.

The term “non-AAV helper function” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “non-AAV helper function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid. In particular, it has been demonstrated that the full-complement of adenovirus genes are not required for accessory helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, EIA and E4 regions are likely required for AAV replication, either directly or indirectly. Laughlin et al., (1982). J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra, Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

In another embodiment, the methods provided herein are carried out with any mammalian cell type which allows for replication of AAV or production of biologic products, and which can be maintained in culture. In one embodiment, mammalian cells used can be HEK293, HeLa, CHO, NSO, SP2/0, PER.C6, Vero, RD, BHK, HT 1080, A549, Cos-7, ARPE-19, and MRC-5 cells.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, NJ (1995); O'Reilly et al., BACULOVIRUS EXPRESSION VECTORS, A LABORATORY MANUAL, Oxford Univ. Press (1994); Samulski et al., J. Vir. (1989) vol. 63, pp. 3822-3828; Kajigaya et al., Proc. Nat'l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). In some embodiments, the nucleic acid construct encoding AAV in insect cells is an insect cell-compatible vector. “Expression vector” refers to a vector including a recombinant polynucleotide including expression control sequences operatively linked to a nucleotide sequence to be expressed. An expression vector includes sufficient cis-acting elements for expression; other elements for expression can be supplied by the host cell or in vitro expression system. Expression vectors include all those known in the art, such as cosmids, plasmids (e.g., naked or contained in liposomes), artificial chromosomes, and viruses that incorporate the recombinant polynucleotide. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In a more preferred embodiment, the vector is a baculovirus, i.e., the construct is a baculoviral vector. Baculoviral vectors and methods for their use are described in the above cited references on molecular engineering of insect cells.

For example, the insect cell line used can be from Spodoptera frugiperda, such as SF9, SF21, SF900+, drosophila cell lines, mosquito cell lines, e.g., Aedes albopictus derived cell lines, domestic silkworm cell lines, e.g., Bombyx mori cell lines, Trichoplusia ni cell lines such as High Five cells or Lepidoptera cell lines such as Ascalapha odorata cell lines. In one embodiment, insect cells are cells from the insect species which are susceptible to baculovirus infection, including High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5 and Ao38.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori nucleopolyhedrovirus (BmNPV) (Kato et al., Appl. Microbiol. Biotechnol. 85(3): 459-70 (2010).

Baculoviruses are commonly used for the infection of insect cells for the expression of recombinant proteins. In particular, expression of heterologous genes in insects can be accomplished as described in for instance U.S. Pat. No. 4,745,051; EP 127,839; EP 155,476; Vlak et al., J. Gen. Virol. 68: 765-76 (1988); Miller et al., Ann. Rev. Microbiol. 42: 177-9 (1988); Carbonell et al., Gene, 73(2): 409-18 (1998); Maeda et al., Nature, 315: 592-4 (1985); Lebacq-Veheyden et al., Molec. Cell. Biol. 8(8): 3129-35 (1988); Smith et al., PNAS, 82: 8404-8 (1985); and Miyajima et al., Gene, 58: 273-81 (1987). Numerous baculovirus strains and variants and corresponding permissive insect host cells that can be used for protein production are described in Luckow et al., Nat. Biotechnol. 6: 47-55 (1988); Maeda et al., Nature, 315: 592-4 (1985); and McKenna et al., J. Invert. Pathol. 71(1): 82-90 (1998).

The baculovirus shuttle vector or bacmids are used for generating baculoviruses. Bacmids propagate in bacteria such as Escherichia coli as a large plasmid. When transfected into insect cells, the bacmids generate baculovirus. In another embodiment, the methods provided herein are carried out with any mammalian cell type which allows for replication of AAV or production of biologic products, and which can be maintained in culture. In one embodiment, mammalian cells used can be HEK293, HeLa, CHO, NSO, SP2/0, PER.C6, Vero, RD, BHK, HT 1080, A549, Cos-7, ARPE-19, and MRC-5 cells.

rAAV particles can also be produced using methods disclosed in various embodiments. In some instances, rAAV particles can be produced by using an insect or mammalian cell that stably expresses some of the necessary components for rAAV particle production. For example, a plasmid (or multiple plasmids) including AAV rep and cap genes, and a selectable marker, such as a neomycin resistance gene, can be integrated into the genome of the cell. In another example, a plasmid (or multiple plasmids) including a selectable marker, such as a neomycin resistance gene, can be integrated into the genome of the cell. The insect, fungal, or mammalian cell can then be co-infected with a helper virus (e.g., adenovirus or baculovirus providing the helper functions) and the viral vector including the 5′ and 3′ AAV ITR (and the nucleotide sequence encoding the heterologous protein, if desired). The advantages of this method are that the cells are selectable and are suitable for large-scale production of the rAAV. As another non-limiting example, adenovirus or baculovirus rather than plasmids can be used to introduce a host regulatory gene, rep gene, and cap gene into packaging cells.

In one embodiment, following an expansion of transfected cells in suspension cell culture through a series of increasingly large culture platforms, a suspension of transfected cells is purified through a multi-step process to remove process impurities, including recombinant baculoviruses and host cells, and enrich for the virions comprising the recombinant parvoviral (rAAV) vector construct. In another embodiment, method provided herein may comprise the step of affinity-purification of the rAAV vector construct using an anti-AAV antibody, in one embodiment an immobilized antibody. In another embodiment, the anti-AAV antibody is a monoclonal antibody. One antibody for use herein is a single chain camelid antibody or a fragment thereof as e.g., obtainable from camels or llamas (see e.g., Muyldermans, Biotechnol. 74: 277-302 (2001). The antibody for affinity-purification of rAAV is an antibody that specifically binds an epitope on an AAV capsid protein, whereby in one embodiment the epitope is an epitope that is present on capsid protein of more than one AAV serotype. For example, the antibody may be raised or selected on the basis of specific binding to AAV5 capsid but at the same time also it may also specifically bind to AAV1, AAV2, AAV3, AAV6, AAV8 or AAV9 capsids.

The methods provided herein for producing rAAV particles produce a population of rAAV particles. In some embodiments, the population is enriched for particles comprising full length or nearly full-length vector genomes by steps that reduce the number of empty capsids.

The population of rAAV particles produced by the methods provided herein are used, for example, for administration in any of the treatment methods described herein.

Host Organism and/or Cells

In a further embodiment, a host cell is provided comprising the vector described above. In one embodiment, the vector construct is capable of being replicated, or capable of expressing the nucleic acid molecule provided herein in the host cell. In some embodiments, provided herein are HCM therapeutics that are host cells comprising a vector construct comprising a nucleic acid encoding cMyBP-C, for use in HCM cell therapy. The cells may be autologous or allogeneic to the subject.

As used herein, the term “host” refers to organisms and/or cells which harbor a nucleic acid molecule or a vector construct of the present disclosure, as well as organisms and/or cells that are suitable for use in expressing a recombinant gene or protein. It is not intended that the present disclosure be limited to any particular type of cell or organism. Indeed, it is contemplated that any suitable organism and/or cell will find use herein as a host. A host cell may be in the form of a single cell, a population of similar or different cells, for example in the form of a culture (such as a liquid culture or a culture on a solid substrate), an organism or part thereof. In one embodiment, a host cell may permit the expression of a nucleic acid molecule provided herein. Thus, the host cell may be, for example, a bacterial, a yeast, an insect or a mammalian cell, or a human cell.

In another embodiment, provided is a means for delivering a nucleic acid provided herein into a broad range of cells, including dividing and non-dividing cells. The present disclosure may be employed to deliver a nucleic acid provided herein to a cell in vitro, e.g. to produce a polypeptide encoded by such a nucleic acid molecule in vitro or for ex vivo gene therapy.

The nucleic acid molecule, vector construct, cells and methods/use of the present disclosure are additionally useful in a method of delivering a nucleic acid provided here into a host, typically a host suffering from HCM.

### Pharmaceutical Formulations

In one embodiment, provided is a pharmaceutical composition comprising a nucleic acid or a vector provided herein and a pharmaceutically acceptable diluent, excipient, or carrier. The pharmaceutical composition may further comprise a second therapeutic agent, or adjuvant, etc. Preferably the composition is sterile if meant for parenteral administration. Preferably the composition is free of infectious viruses and toxins. Preferably the composition is stable for a suitable period of time under storage conditions.

By “pharmaceutically acceptable” it is meant a material that is not biologically or otherwise undesirable, i.e., the material may be administered to a subject without causing any undesirable biological effects. Thus, such a pharmaceutical composition may be used, for example, in transfection of a cell ex vivo or in administering a viral particle or cell directly to a subject.

A carrier may be suitable for parenteral administration, which includes intravenous, intraperitoneal or intramuscular administration. Alternatively, the carrier may be suitable for sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions provided herein is contemplated.

In other embodiments, provided herein are pharmaceutical compositions (i.e., formulations) of AAV particles useful for administration to subjects suffering from a genetic disorder to deliver gene encoding a protein of interest. In certain embodiments, the pharmaceutical formulations provided herein are liquid formulations that comprise recombinant AAV particles comprising any of the vector constructs disclosed herein. The concentration of recombinant AAV virions in the formulation may vary.

In other embodiments, the AAV particle pharmaceutical formulation provided herein comprises one or more sterile pharmaceutically acceptable excipients to provide the formulation with advantageous properties for storage and/or administration to subjects for the treatment of the genetic disorder.

In certain aspects, the formulation comprising recombinant AAV particle further comprises one or more buffering agents.

In another embodiment, the recombinant AAV particle formulation provided herein may comprise one or more isotonicity agents, such as sodium chloride. Other buffering agents and isotonicity agents known in the art are suitable and may be routinely employed for use in the formulations provided herein.

In another embodiment, the recombinant AAV particle formulations provided herein may comprise one or more bulking agents. Exemplary bulking agents include without limitation mannitol, sucrose, dextran, lactose, trehalose, and povidone (PVP K24).

In yet another embodiment, the recombinant AAV particle formulations provided herein may comprise one or more surfactants, which may be non-ionic surfactants. Exemplary surfactants include ionic surfactants, non-ionic surfactants, and combinations thereof. For example, the surfactant can be, without limitation, TWEEN 80 (also known as polysorbate 80, or its chemical name polyoxyethylene sorbitan monooleate), sodium dodecylsulfate, sodium stearate, ammonium lauryl sulfate, TRITON AG 98 (Rhone-Poulenc), poloxamer 407, poloxamer 188 and the like, and combinations thereof.

The recombinant AAV particle formulations provided herein are typically sterile and stable and can be stored for extended periods of time without an unacceptable change in quality, potency, or purity.

In some embodiments, isotonic agents, for example, sugars, polyalcohols such as mannitol, sorbitol, or sodium chloride are included in the composition. Prolonged absorption of the injectable compositions can be brought about by including in the composition an agent which delays absorption, for example, monostearate salts and gelatin. In certain embodiments, a nucleic acid or vector construct provided herein may be administered in a time or controlled release formulation, for example in a composition which includes a slow release polymer or other carriers that will protect the compound against rapid release, including implants and microencapsulated delivery systems. Biodegradable, biocompatible polymers may for example be used, such as ethylene vinyl acetate, polyanhydrides, polyglycolic acid, collagen, polyorthoesters, polylactic acid and polylactic, polyglycolic copolymers (PLG).

In certain embodiments, the pharmaceutical composition comprising the vector construct or AAV particle provided herein may be of use in transferring genetic material to a cell. Such transfer may take place in vitro, ex vivo or in vivo. Accordingly, one embodiment provides a method for delivering a nucleotide sequence to a cell, which method comprises contacting a nucleic acid, a vector construct, or a pharmaceutical composition as described herein under conditions such the nucleic acid or vector provided herein enters the cell. The cell may be a cell in vitro, ex vivo or in vivo.

### Methods of Treatment

The vector constructs or AAV particles described herein are administered to subjects in a dose effective to deliver a MYBPC3 gene to the heart of a mammalian subject. The subject is preferably a human, including a juvenile subject. Juvenile subjects may range in age from 0-2, 2-6, 2-10, 2-12, 2-15, 2-18, 12-18, or 0-18 years of age, for example.

Such methods include methods of expressing cMyBP-C in heart of a mammalian subject comprising administering to the subject an effective amount of a composition comprising the vector construct described herein, the rAAV particle described herein, or the pharmaceutical composition described herein, thereby expressing cMyBP-C in the heart tissue (e.g., myocardium, or myocardiocytes) of the subject.

Such methods also include a method of treating a deficiency in functional wild type myosin binding protein C in a mammalian subject by administering an amount of the vector construct, rAAV particle or pharmaceutical composition effective to increase the level of functional myosin binding protein C in the heart tissue (e.g., myocardiocytes). In one or more embodiments, such methods increase levels of cMyBP-C expression in the heart, by at least 50%, at least 60%, at least 70%, at least 80%, at least 90%, at least 95%, or at least 98% compared to the levels without treatment, or to the levels seen in healthy humans. In some embodiments, the amount of the vector construct, rAAV particle or pharmaceutical composit84hosphatifective to increase the level of myosin binding protein C in heart tissue (e.g., myocardiocytes) by at least about 2-fold, and/or to restore contractile force, relative tension, calcium-activated tension, relaxation time, in engineered heart tissue in vitro or in animal tissue in vivo.

Such methods also include a method of treating HCM in a mammal, or treating or preventing any symptom thereof, comprising administering a therapeutically effective amount of the vector construct, rAAV particle or pharmaceutical composition. In such methods, the mammal may have a mutation in one or both alleles of the cMyBP-C gene. Such methods, for example, reduce heart size, reduce cardiothoracic ratio, reduce end diastolic or end systolic left ventricular diameter, reduce anterior or posterior wall thickness, increase ejection time, increase aortic peak flow velocity or aortic flow time, and/or decrease symptoms of disease. In one or more embodiments, such methods reduce the frequency or severity of symptoms such as heart failure, arrhythmias, chest pain, shortness of breath, fatigue and dizziness.

In any of the methods described herein, the rAAV particle is delivered at a dose of about 1e12 to about 6e14 vg/kg in an aqueous suspension.

In any of the methods described herein, the administration of the vector construct, rAAV particle, or pharmaceutical composition may further comprise administration of prophylactic or therapeutic corticosteroid treatment, and/or may further include administration of a second therapeutic agent for treating HCM including but not limited to beta, blockers, calcium channel blockers, anti-arrhythmia drugs and small molecule inhibitors of cardiac myosin.

In any of the methods herein, prior to administration of an AAV particle to a patient as described above, the prospective patient may be assessed for the presence of anti-AAV capsid antibodies or anti-AAV neutralizing antibodies that are capable of blocking cell transduction or otherwise reduce the overall efficiency of the treatment.

### Detection of Anti-AAV Antibodies

To maximize the likelihood of successful cardiac transduction with systemic AAV-mediated therapeutic gene transfer, prior to administration of an AAV particle in a therapeutic regimen to a human patient as described above, the prospective patient may be assessed for the presence of anti-AAV capsid antibodies or anti-AAV neutralizing antibodies that are capable of blocking cell transduction or otherwise reduce the overall efficiency of the therapeutic regimen. Such antibodies may be present in the serum of the prospective patient and may be directed against an AAV capsid of any serotype. In one embodiment, the serotype against which pre-existing antibodies are directed is AAV5.

Methods to detect pre-existing AAV immunity are well known and routinely employed in the art and include cell-based in vitro transduction inhibition (TI) assays, in vivo (e.g., in mice) TI assays, and ELISA-based detection of total anti-capsid antibodies (tAb) (see, e.g., Masat et al., Discov. Med., vol. 15, pp. 379-389 and Boutin et al., (2010) Hum. Gene Ther., vol. 21, pp. 704-712). TI assays may employ host cells into which an AAV-inducible reporter vector has been previously introduced. The reporter vector may comprise an inducible reporter gene such as GFP, etc. whose expression is induced upon transduction of the host cell by an AAV virus. Anti-AAV capsid antibodies present in human serum that are capable of preventing/reducing host cell transduction would thereby reduce overall expression of the reporter gene in the system. Therefore, such assays may be employed to detect the presence of anti-AAV capsid antibodies in human serum that are capable of preventing/reducing cell transduction by the therapeutic AAV particle.

The assays to detect anti-AAV capsid antibodies may employ solid-phase-bound AAV capsid as a “capture agent” over which human serum is passed, thereby allowing anti-capsid antibodies present in the serum to bind to the solid-phase-bound capsid “capture agent”. Once washed to remove non-specific binding, a “detection agent” may be employed to detect the presence of anti-capsid antibodies bound to the capture agent. The detection agent may be an antibody, an AAV capsid, or the like, and may be detectably-labeled to aid in detection and quantitation of bound anti-capsid antibody. In one embodiment, the detection agent is labeled with ruthenium or a ruthenium-complex that may be detected using electrochemiluminescence techniques and equipment.

The same above-described methodology may be employed to assess and detect the generation of an anti-AAV capsid immune response in a patient previously treated with a therapeutic AAV virus of interest. As such, not only may these techniques be employed to assess the presence of anti-AAV capsid antibodies prior to treatment with a therapeutic AAV virus, they may also be employed to assess and measure the induction of an immune response against the administered therapeutic AAV virus after administration. As such, contemplated herein are methods that combine techniques for detecting anti-AAV capsid antibodies in human serum and administration of a therapeutic AAV virus for the treatment of HCM, wherein the techniques for detecting anti-AAV capsid antibodies in human serum may be performed either prior to or after administration of the therapeutic AAV virus.

Other aspects and advantages of the present disclosure will be understood upon consideration of the following illustrative examples.

### EXAMPLES

### Example 1: Production of AAV Particles

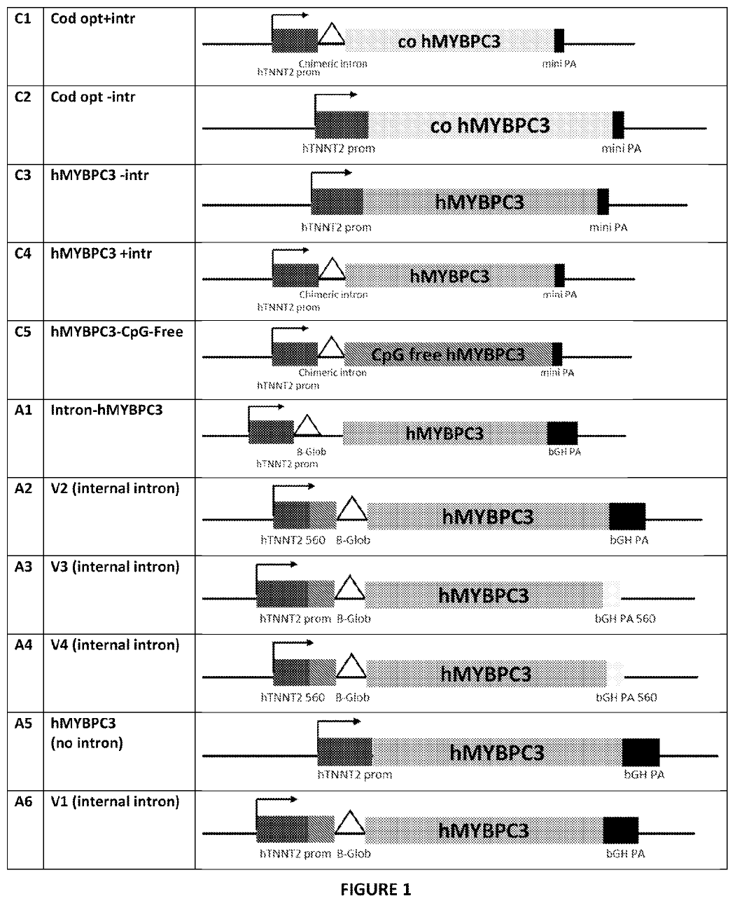

FIG. 1 shows the organization of the elements of vector constructs designated A1-A8 and C1-C5 SEQ ID NOS: 3-41 or 92-169, respectively, that comprise nucleic acid encoding human cMyBP-C. AAV particles comprising AAV9 capsid and vector constructs of SEQ ID NOS: 3-26 were produced in HEK293 cells and Sf9 cells. Vectors for AAV productions were generated. For example in AAV production with HEK293 cells, plasmids were generated. These plasmids have nucleotide sequences that provide the AAV vector genome, encode Rep and Capsid proteins, and provide non-helper AAV functions. These plasmids were transfected into HEK293 cells using a transfection reagent. After allowing the HEK293 to culture after transfection for a predetermined time, the produced rAAV particles were isolated from the culture, purified, and titered. For AAV production in Sf9 cells, bacmids were generated. These bacmids have nucleotide sequences that provide the AAV vector genome and encode Rep and Capsid proteins. The bacmids were transfected into naive Sf9 cells using a transfection reagent. After allowing the transfected Sf9 cells to culture for a predetermined time, recombinant baculovirus (rBV) was isolated, purified, and titered. To produce rAAV, another naive culture of Sf9 cells were infected with the rBV at a predetermined multiplicity of infection (MOI). The Sf9 cells were allowed to culture for a predetermined time post infection. After the predetermined time, the produced rAAV particles were isolated from the culture, purified, and titered.

### Example 2: Evaluation of Effect of AAV Particles in Engineered Heart Tissue

AAV9 particles prepared using the MYBPC3 vector constructs described herein were analyzed for their effect on human iPSC-derived cardiomyocytes in 2D and 3D formats. In the 2D format, exogenous protein and RNA content were measured, as well as suppression of mutant MYBPC3 transcripts (see Example 5 below). In the 3D format, effect on contractile function was also measured, including beating frequency, contractile force and kinetics.

A patient-specific human induced pluripotent stem cell (hiPSC) line carrying an heterozygous MYBPC3 truncating mutation was used to create, by CRISPR/Cas9 genome editing, two hiPSC lines: 1) cpHet carrying an additional homozygous MYBPC3 truncating mutation which lead to complete absence of MYBPC3 protein; and 2) isogenic control carrying two wild-type alleles leading to a normal level of MYBPC3 protein. See Warnecke et al., Generation of bi-allelic MYBPC3 truncating mutant and isogenic control from an iPSC line of a patient with hypertrophic cardiomyopathy. Stem Cell Res. 55 (2021): 102489.

AAV9 particles comprising the vector constructs described herein were tested by contact with human cardiomyocytes (CM) derived from the cpHet hiPSC line and the isogenic control hiPSC line, in 2D and engineered heart tissue (EHT) formats.

The hiPSC cardiomyocytes (hiPSC-CM) were prepared by passaging the cpHet or isogenic control hiPSC cells, dissociating the hiPSC cells, and boosting cardiac differentiation with the use of different media over 14 days. The differentiated cardiomyocytes were cultured in a 2D monolayer or as 3D engineered heart tissue (EHT). EHTs were prepared by embedding 1 Million hiPSC-CMs into a fibrin matrix generally as described in Breckwoldt K, et al. Differentiation of cardiomyocytes and generation of human engineered heart tissue. Nat Protoc 12, 1177-1197 (2017) and Hansen et al., Development of a drug screening platform based on engineered heart tissue. Circ. Res. 107.1 (2010): 35-44.

Briefly, slots for EHTs with spacers are placed in a 24-well plate filled with 2%-agarose (1.6 ml/well). When the agarose is solid, the spacers are removed and the silicone racks placed with one pair of posts into each mold. For each EHT casting, approximately 1 million hiPSC-CMs are used in a master medium containing horse serum, Y-27632 (Biorbyt, orb154626), fibrinogen, L-glutamine, DMEM, penicillin/streptomycin and thrombin. The hiPSC-CMs and casting medium are placed in the agarose slots. After 1.5 hours, EHTs solidify around the silicone racks and are moved to medium-filled culture plates. EHTs are then maintained at 37° C., 7% CO₂, 40% O₂ and 98% relative humidity.

For transduction with AAV, HiPSC-CM in 2D were re-plated and cultured as adherent cells with 220K cells per 24 well plate or 20K cells per 96 well plate. 2D cpHet hiPSC-CM were transduced about 4 days after re-plating by addition of AAV particles (at a multiplicity of infection (MOI) of 300K) into the culture medium. cpHet EHT were transduced about 2 weeks after generation by addition of AAV particles (multiplicity of infection (MOI) of 300K) into the tissue medium.

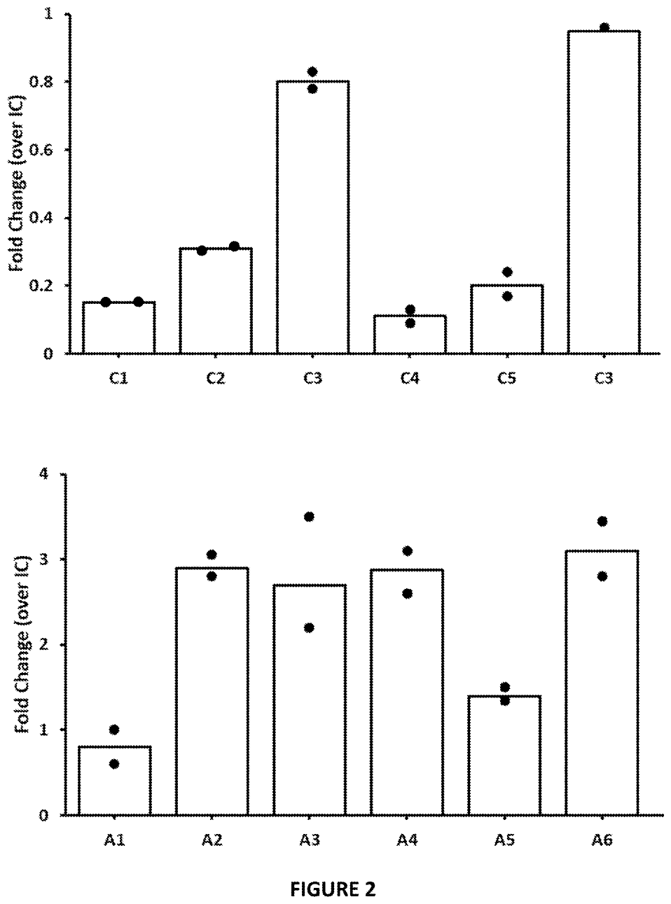

The expression of human cMyBP-C protein was detected 7 or 14 days after transduction for 2D and 3D EHT formats, respectively. Briefly, 2D or EHT hiPSC-CM were harvested to extract proteins for further analysis. Whole protein lysates were used to measure exogenous protein levels by Western blot utilizing custom-made antibody against hMYBPC3. Alpha-actinin and/or cTnT protein level was used as reference. No MYBPC3 was detected in untransduced cpHet hiPSC-CMs. After transduction with AAV9 particles, exogenous MYBPC3 protein was detected, and protein levels were depicted as fold-increase over isogenic control. Results are shown in FIG. 2 and show that at least a 2.5-fold increase in expression was achieved with A2, A3, A4, A6, with A6 achieving the highest increase, and at least a 0.1-1.0-fold increase in expression was achieved with C1, C2, C4 and C5, with C3 achieving the highest increase. Testing of additional AAV9 particles comprising vectors constructs described herein, produced in HEK293 cells or in Sf9 derived cells, resulted in an even higher 5-12 fold-change over isogenic control as analyzed by Western blot.

In the 3D EHT format, the effect on spontaneous beating frequency and on contractile function was evaluated after administration of the AAV9-MYBPC3 vector constructs described herein. For evaluating contractile function, the engineered heart tissue was stimulated to contract and relax. The hiPSC-CMs in EHT format exhibit intrinsic spontaneous contractility starting 1-2 weeks after generation. For spontaneous beating frequency as functional endpoint, the intrinsic spontaneous contraction frequency was analyzed without further stimulation.

Spontaneous beating activity was evaluated in low glucose DMEM medium with 1.8 mM calcium and was measured 1 week post transduction. Higher spontaneous beating frequency was consistently observed in cpHet EHTs compared to isogenic control EHT. The goal of gene therapy with AAV9 particles containing the vector constructs described herein was to reduce this abnormally high frequency. The frequency was analyzed over time in complete medium, and in serum-free DMEM one and two weeks after transduction. Constructs A2, A3, A4, A5 and A6 resulted in reversion of abnormal phenotype to normal phenotype. Specifically, constructs A3, A4 and A6 showed the best and most consistent decrease in beating rate (BPM). In contrast, construct A5 showed only a slight effect on beating rate.

Contractile function was evaluated at a fixed beating frequency by pacing using electrical stimulation. See Hirt et al., Functional improvement and maturation of rat and human engineered heart tissue by chronic electrical stimulation. J. Molec. Cell. Cardiol. 74 (2014): 151-161. One or 2 weeks after transduction, short term electrical pacing (1 Hz, 2.5 V) in medium containing 1.8 mM calcium was employed to evaluate the endpoints under fixed frequencies with Baseline Force as confirmatory QC endpoint, and contraction kinetics as functional endpoints (relative late relaxation time, RT_{20%} and RT_{80%}). The transduction procedure was optimized to provide stable force development with no significant difference between cpHet non-transduced (NT) and EHTs transduced with vectors by adjusting time point, transduction medium and transduction chambers.

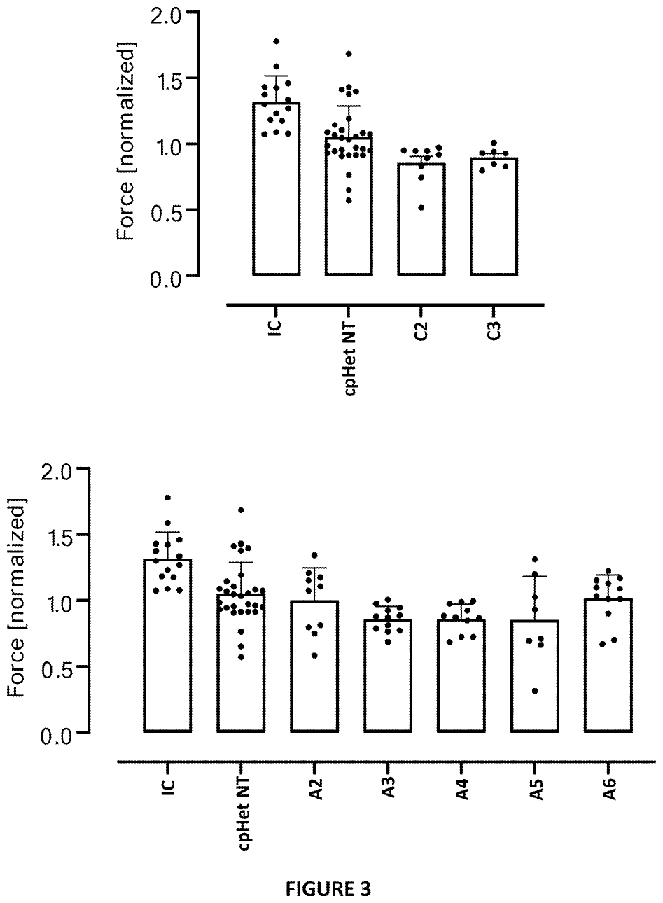

After activation, the contractile force of the myocytes in engineered heart tissue was measured. Contractile force and kinetics of EHTs was monitored in the EHT test system combining a semi-automated video optical analysis with a figure recognition software. The force (mN) development over time (sec) was calculated by the delta of silicone post distance (post deflection) with known elastic modulus of Sylgard 184 (2.6 kPa). For depiction of normalized average peaks, baseline was set to 0% and peak force to 100% for each EHT. Non-transduced (NT) cpHet EHTs were compared with transduced cpHet and isogenic control EHTs and normalized. Results are shown in FIG. 3.

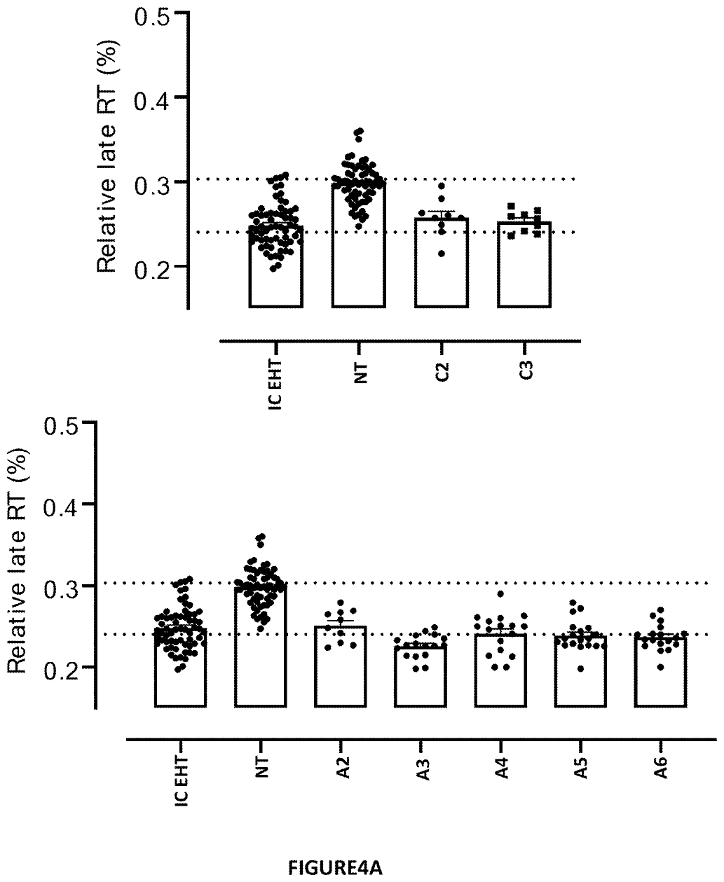

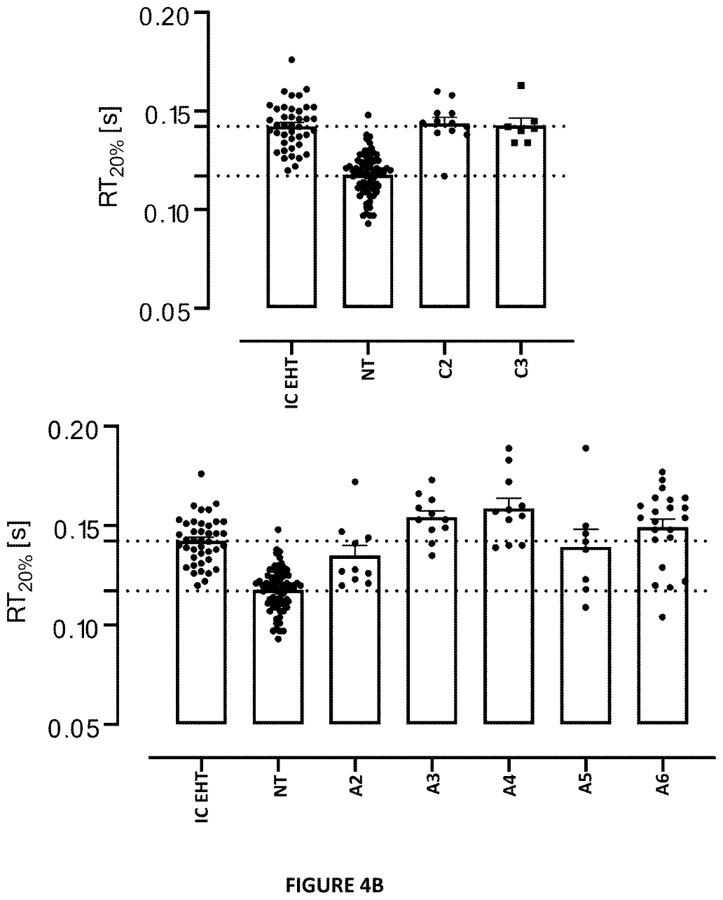

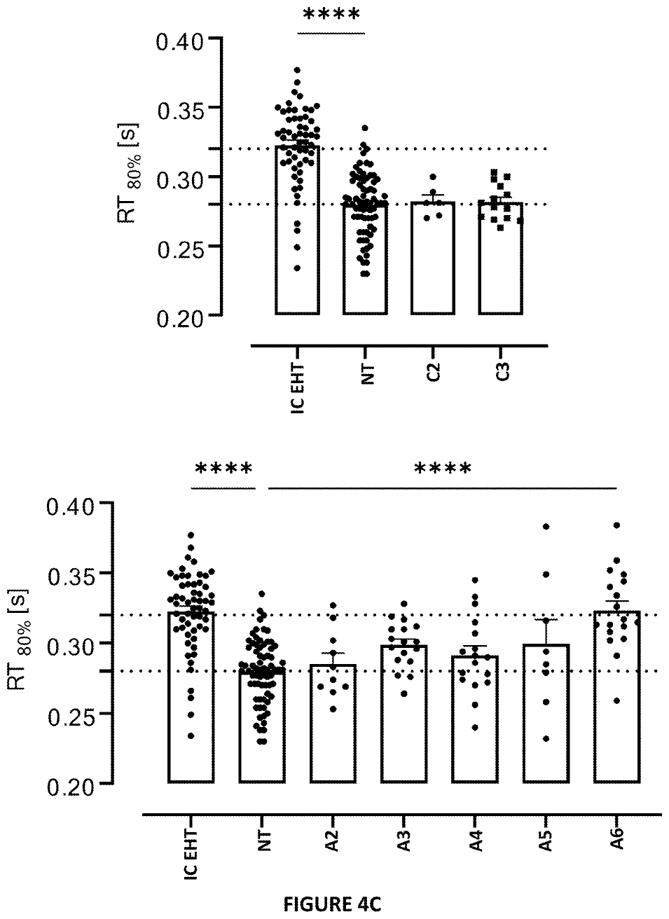

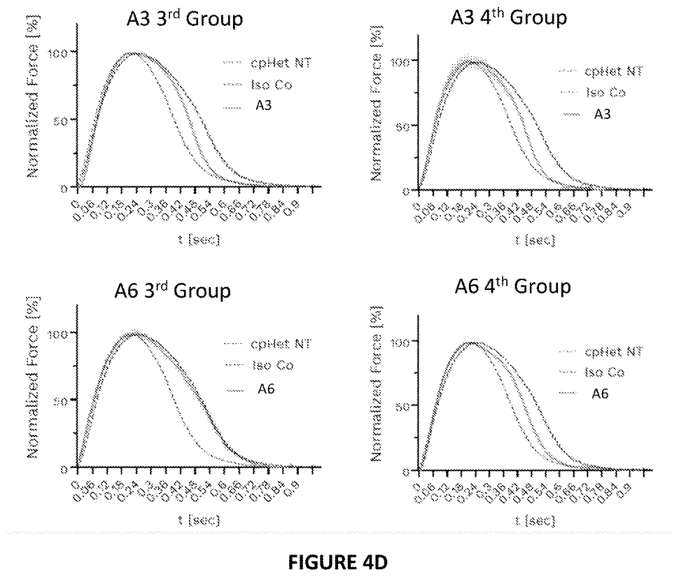

The relaxation time and rate were measured from peak sarcomere contraction to re-lengthening. Recorded contractions are identified by peak criteria. Based on identified contractions, values for frequency, average force, fractional shortening, contraction (Time to peak)—and relaxation time (RT) were calculated. Relaxation time percentage represents time needed from maximum post deflection to baseline in percentage, e.g., with RT20% representing the time (sec) from maximum (100%) to 80% post deflection. From absolute RTs, relative late RT (fraction or %)=RT80%−RT50%/RT80% was calculated. The relative percentage of late relaxation time is shown in FIG. 4A. The relaxation time to 20% or 80% of re-lengthening (seconds) is shown in FIGS. 4B and 4C, respectively. cpHet show a consistently higher fraction of relative late RT (RT80%−RT50%/RT80%) compared to isogenic control. The goal of gene therapy is to normalize contractile kinetics, including by lowering the abnormally high relative late RT.

The effect on contractile function was assessed for AAV9 particles containing various MYBPC3 vector constructs that were produced in HEK293 cells, Sf9 and Sf9 derived cells. cpHet EHTs transduced with AAV9-MYBPC3 were compared I week after transduction with time-matched isogenic control EHTs. Most of the tested vector constructs, for example, A2, A3, A4, A5, A6 and C3, reverted the abnormally high relative late RT to closer to normal phenotype. The effect on contractile function of AAV9 particles produced in HEK293 cells was superior to particles produced in Sf9 cells. A3, A4, A5 and A6 had the greatest effect and completely reverted the abnormally high relative late RT.

Absolute relaxation time was also evaluated. cpHet EHTs exhibit a significantly shorter absolute relaxation time for both 20% and 80% (RT_{20%} and RT_{80%}) compared to isogenic control. Tested vector constructs, for example, A2, A3, A4, A5, A6 and C3 significantly lengthened the abnormally short RTs. Treatment of cpHet EHT cardiomyocytes with AAV particles produced in HEK293 cells showed the most apparent restoration for both (RT_{20%} and RT_{80%}) compared to non-transduced cpHet EHT cardiomyocytes. Construct A6 had a statistically significant effect on both RT_{20%} and RT_{80%}. Vector A6-transduced EHTs exhibited both a significantly longer RT_{20%} and a longer RT_{80%}.

Additionally, normalized average contraction peaks of AAV-transduced cpHet EHT were analyzed and compared to normalized average contraction peaks from cpHet and isogenic control EHTs. Some effects were seen for all test vectors, e.g., A2, A3, A4, A5, A6 and C3 consistently having the greatest effect. AAV9 particles comprising vector construct A6 produced in HEK293 cells resulted in complete normalization of contractile kinetics. cpHet EHTs transduced with AAV9 particles containing vector constructs A3 and A6, produced in HEK293 derived cells, improved relaxation kinetics towards the isogenic control. As shown in FIG. 4D, the normalization force % of cells transduced with A3 and A6 produced in HEK293 cells (Group 3) was significantly greater than the normalization force % of cells transduced with A3 and A6 produced in insect cells (Group 4). Overall, AAV9 particles produced in mammalian cells showed superior activity in improving contractile function and contractile kinetics compared to AAV9 particles produced in insect cells.

### Example 3: Evaluation of Effect of AAV Particles In Vivo

Mice (n=10) were administered AAV particles prepared as in Example 1 at doses of 2e14 vg/kg and heart tissue was collected at 8 weeks.

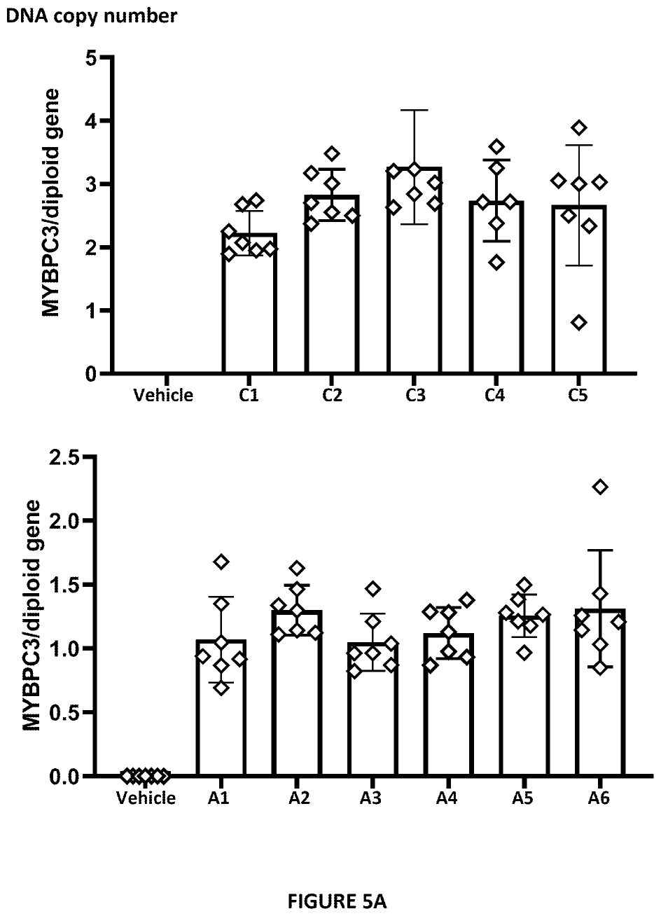

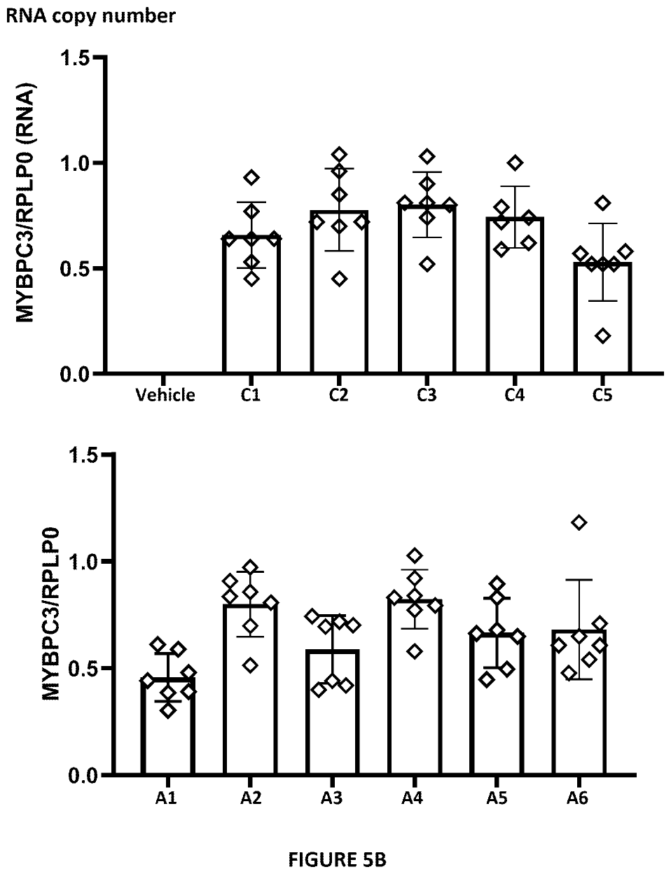

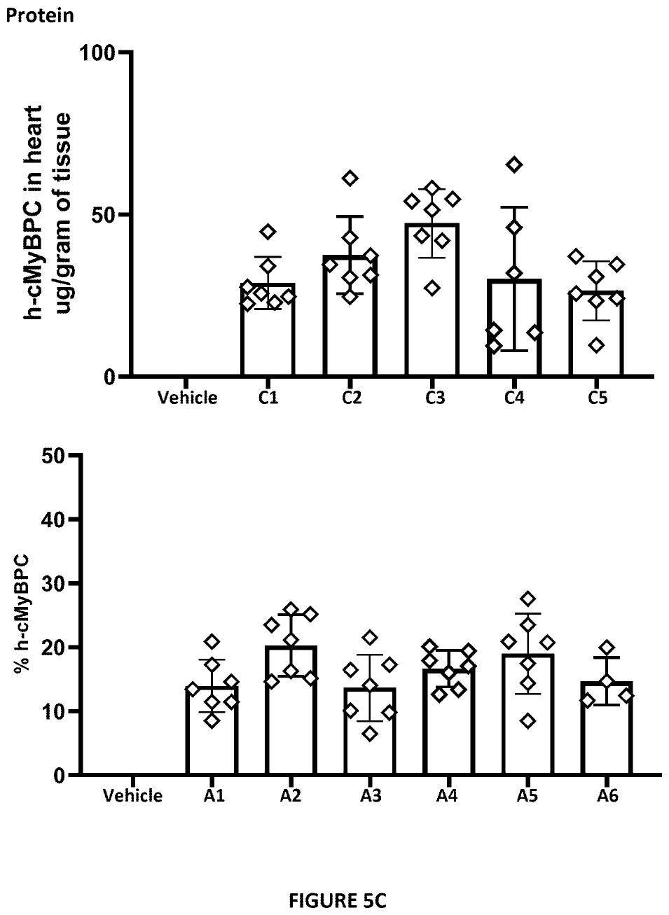

The number of vector genomes encoding human cMyBP-C was assessed by ddPCR. Results (vector genomes per number of diploid gene) are shown in FIG. 5A. All of the AAV particles tested provided effective delivery of at least one copy of the gene encoding human cMyBP-C per cell.

The number of human cMyBP-C mRNA transcripts was assessed by ddPCR. Results (mRNA transcripts per RPLP0 ribosomal protein transcript) are shown in FIG. 5B. All of the AAV particles tested provided effective translation into mRNA, with A2 and A4 providing highest levels.

The amount of cMyBP-C protein was determined by liquid chromatography/mass spectrometry (LC/MS). Results (ug/gram of heart tissue and percentage of human cMyBP-C of total cMyBP-C protein in murine heart) are shown in FIG. 5C.

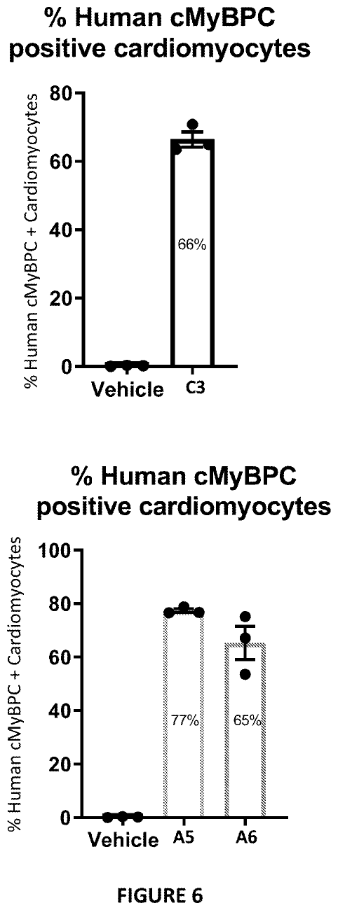

Heart tissue was stained with antibodies specific for human cMyBP-C and ASG (a-sarcoglycan, a muscle cell membrane marker). Intact cell nuclei were also stained with DAPI. There was widespread detection of cMyBP-C protein throughout a majority of cardiomyocytes, with 77% and 65% of cardiomyocytes positive for cMyBP-C in preparations from mice administered A5, A6 and C3, respectively. See FIG. 6.

Heart tissue was also stained with antibodies specific for human cMyBP-C and for actin which is present in the sarcomeres. The human cMyBP-C protein was observed to localize to the sarcomere.

The results indicate that A5 and A6 vector constructs provided effective delivery of human cMyBP-C protein to the mice administered AAV particles comprising these vector constructs, and that the human cMyBP-C protein was effectively incorporated into the majority of cardiomyocyte sarcomeres. The integration of the functional human cMyBP-C protein is expected to improve contractility and reduce hypertrophic cardiomyopathy and its associated symptoms.

### Example 4: Further Evaluation of Effect of AAV Particles In Vivo

Various doses of rAAV particles comprising the vector constructs described herein are administered to MYBPC3 KO mice to evaluate correction of a HCM phenotype which includes hypertrophy and cardiac dysfunction. Echocardiography will be used to monitor the functional correction of these mice throughout the length of the study and upon study completion, heart tissues will be used to evaluate transduction and expression of the vector construct.

### Example 5: Evaluation of Mutant MYBPC3 mRNA Levels

The effect of AAV9 particles containing vector constructs described herein on mutant MYBPC3 mRNA levels was also evaluated in 2D hiPSC-CM prepared as described in Example 2.

Semi-quantitative RT-PCR was performed to monitor effect on mutant MYBPC3 mRNAs. RT-PCR with primers around the MYBPC3 c.2308G>A mutation site showed only one band at 912 bp in the isogenic control CM, corresponding to the wild-type mRNA, and two additional mRNA bands of bigger size compared to wild-type band (i.e., 912 bp) in the non-transduced CpHet CM. The accumulation of these two aberrant MYBPC3 mRNAs resulting from the endogenous mutated gene was prevented by transduction with the AAV9-MYBPC3 vector constructs as described herein, e.g., A1, A2, A3, A4, A5, A6. C1, C4 and C5 had the least effect on the mutant mRNA bands.

Cardiovascular safety and toxicology studies will be performed in relevant models. Dose-response studies to determine % transduced/protein expression in MYBPC3−/− mice to inform dose selection will be conducted.

The embodiments described herein are intended to be merely exemplary, and those skilled in the art will recognize, or will be able to ascertain using no more than routine experimentation, numerous equivalents of specific constructs, materials, and procedures. All such equivalents are considered to be within the scope of the disclosure.

All of the patents, patent applications and publications referred to herein are incorporated by reference herein in their entireties. Citation or identification of any reference in this application is not an admission that such reference is available as prior art to this application. The full scope of the disclosure is better understood with reference to the appended claims.

1. A recombinant vector construct comprising:
a nucleic acid encoding a functional human cardiac myosin binding protein C (cMyBP-C) comprising an amino acid sequence at least 95% identical to SEQ ID NO: 2, or complement thereof, operably linked to:
a heterologous cardiomyocyte-specific transcription regulatory region comprising a fragment or variant of a hTNNT2 promoter,
a polyadenylation signal, and
one or both of 5′ and 3′ AAV inverted terminal repeat (ITR) sequences.

2-5. (canceled)

6. The vector construct of claim 1, wherein the cardiomyocyte-specific transcription regulatory region comprises:
a cardiomyocyte-specific promoter comprising a nucleotide sequence at least 80% identical to any one of SEQ ID NOs: 47-52 or a fragment or complement thereof, and
an intron comprising a nucleotide sequence at least 60% identical to any of SEQ ID NOs: 53, 56 or 58 or complement thereof, wherein the intron is 5′ to the nucleic acid encoding a functional human cMyBP-C.

7. The vector construct of claim 6 comprising a fragment of an exon.

8. The vector construct of claim 7, wherein the exon comprises the nucleotide sequence of SEQ ID NO: 54 or complement thereof.

9. The vector construct of claim 7, comprising the nucleotide sequence of SEQ ID NO: 56 or complement thereof.

10. The vector construct of claim 1, further comprising an intron.

11. The vector construct of claim 10, wherein the intron comprises a nucleotide sequence at least 60% identical to SEQ ID NO: 53 or SEQ ID NO: 58 or complements thereof.

12. The vector construct of claim 10, wherein the intron is located within the nucleic acid encoding a functional human cMyBP-C.

13. The vector construct of claim 10, wherein the intron is located between two exons of the nucleic acid encoding a functional human -MyBP-C.

14. The vector construct of claim 10, wherein the intron is located between exon 2 and exon 3 of the nucleic acid encoding a functional human cMyBP-C.

15. The vector construct of claim 10, wherein the intron is located at position 293 of SEQ ID NO: 1 or 42-45.

16-27. (canceled)

28. The vector construct of claim 1, wherein the polyadenylation signal comprises a nucleotide sequence at least 90% identical to SEQ ID NO: 64 or complement thereof.

29. The vector construct of claim 1, wherein the polyadenylation signal is a bovine growth hormone polyadenylation signal or fragment thereof.

30. The vector construct of claim 29, wherein the polyadenylation signal comprises a nucleotide sequence at least 90% identical to any of SEQ ID NOs: 59-61 or complements thereof.

31. The vector construct of claim 1 wherein the polyadenylation signal is a human growth hormone polyadenylation signal or fragment thereof.

32. The vector construct of claim 31, wherein the polyadenylation signal comprises a nucleotide sequence at least 90% identical to any of SEQ ID NOs: 62 or fragment or complement thereof.

33. (canceled)

34. (canceled)

35. The vector construct of claim 1 comprising a nucleotide sequence at least 97%, 98% or 99% identical to any of SEQ ID NO: 3-41 or 92-169 or complements thereof.

36. An rAAV particle comprising the vector construct of claim 1 and an AAV capsid, wherein the AAV capsid has cardiac tropism.

37-40. (canceled)

41. A method of producing an rAAV particle comprising the steps of:
(a) providing a cell permissive for AAV replication with one or more nucleic acid constructs comprising:
(i) a recombinant vector construct comprising (1) at least one AAV ITR, (2) a heterologous cardiomyocyte-specific transcription regulatory region, and (3) a nucleic acid encoding a functional human cardiac myosin binding protein C,
(ii) a nucleotide sequence encoding one or more AAV Rep proteins which is operably linked to a promoter that is capable of driving expression of the Rep protein(s) in the cell; and
(iii) a nucleotide sequence encoding one or more AAV capsid proteins which is operably linked to a promoter that is capable of driving expression of the capsid protein(s) in the cell;
(b) culturing the cell under conditions permitting expression of the Rep and the capsid proteins; and
(c) recovering the rAAV particle.

42-45. (canceled)

46. A pharmaceutical composition comprising the rAAV particle of claim 36 in an aqueous suspension with a sterile pharmaceutically acceptable excipient.

47-49. (canceled)
